SEQUENCE LISTING (156 sequences)

SEQ ID NO: 1 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGAKNLSGQSPARTSSDPKTNTTTEDHKIVASENSSAMVQVHSQGREAAVSHLTTLATISTSPQPPTTKPGPDNSTYNTPVYKLDTSEATQVEQHHRRTDNDSTASDTPPATTAAGHPKAENTNTSKSADSLDPATTTSPPNHSETAGNNNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 2 - protein, 74 aa, Sudan ebolavirus
GLRQLANETTQALQLFLRATTELRTYTILNRKAIDFLLRRWGGTCRILGPDCCIEPHDWTKNITDKINQIIHDF

SEQ ID NO: 3 - protein, 74 aa, Zaire ebolavirus
GLRQLANETTQALQLFLRATTELRTFSILNRKAIDFLLQRWGGTCHILGPDCCIEPHDWTKNITDKIDQIIHDF

SEQ ID NO: 4 - protein, 74 aa, Bundibugyo ebolavirus; identical to SEQ ID NO: 3

SEQ ID NO: 5 - protein, 74 aa, Tai Forest ebolavirus
GLRQLANETTQALQLFLRATTELRTFSILNRKAIDFLLQRWGGTCHILGPDCCIEPQDWTKNITDKIDQIIHDF

SEQ ID NO: 6 - protein, 32 aa, synthetic construct
  MOD_RES 18: note = R8 octenyl alanine
  MOD_RES 25: note = S5 pentenyl alanine
TCHILGPDCAIEPHDWTXNITDKIXQIIHDFV

SEQ ID NO: 7 - protein, 34 aa, synthetic construct
  MOD_RES 34: note = Lys(epsilon-(PEG)4-thiocholesterol)
TCHILGPDCAIEPHDWTKNITDKIDQIIHDFVDX

SEQ ID NO: 8 - protein, 21 aa, synthetic construct
  MOD_RES 2: note = R8 octenyl alanine
  MOD_RES 9: note = S5 pentenyl alanine
  MOD_RES 21: note = Lys (epsilon-(PEG)4-thiocholesterol)
HXWTKNITXKINQIIHDFVNX

SEQ ID NO: 9 - protein, 34 aa, synthetic construct
  MOD_RES 19: note = S5 pentenyl alanine
  MOD_RES 23: note = S5 pentenyl alanine
  MOD_RES 34: note = Lys (epsilon-(PEG)4-thiocholesterol)
TCHILGPDCAIEPHNWTKXITNXINQIIHDFVNX

SEQ ID NO: 10 - protein, 32 aa, unidentified
  source 1..32: note = Ebolavirus
TCHILGPDCAIEPHDWTKNITDKIDQIIHDFV

SEQ ID NO: 11 - protein, 33 aa, synthetic construct
  MOD_RES 33: note = Lys(epsilon-(PEG)4-thiocholesterol)
TCHILGPDCAIEPHDWTKNITDKIDQIIHDFVX

SEQ ID NO: 12 - protein, 33 aa, synthetic construct
  MOD_RES 33: note = PEG(n)-cholesterol or PEG(n)-thiocholesterol, wherein n = 1-36
  MOD_RES 17: note = Alpha, Alpha-disubstituted non-natural amino acid with olefinic side chain
  MOD_RES 24: note = Alpha, Alpha-disubstituted non-natural amino acid with olefinic side chain
  CROSSLNK 17..24: note = The residues are cross-linked to each other
TCHILGPDCAIEPHDWXKNITDKXDQIIHDFVX

SEQ ID NO: 13 - protein, 33 aa, synthetic construct
  MOD_RES 18: note = Alpha, Alpha-disubstituted non-natural amino acid with olefinic side chain
  MOD_RES 25: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDKIXQIIHDFVX

SEQ ID NO: 14 - protein, 33 aa, synthetic construct
  MOD_RES 19: note = Alpha, Alpha-disubstituted non-natural amino acid with olefinic side chain
  MOD_RES 26: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 19..26: note = The residues are cross-linked to each other
TCHILGPDCAIEPHDWTKXITDKIDXIIHDFVX

SEQ ID NO: 15 - protein, 33 aa, synthetic construct
  MOD_RES 20: note = Alpha, Alpha-disubstituted non-natural amino acid with olefinic side chain
  MOD_RES 27: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 20..27: note = The residues are cross-linked to each other
TCHILGPDCAIEPHDWTKNXTDKIDQXIHDFVX

SEQ ID NO: 16 - protein, 33 aa, synthetic construct
  MOD_RES 21: note = Alpha, Alpha-disubstituted non-natural amino acid with olefinic side chain
  MOD_RES 28: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 21..28: note = The residues are cross-linked to each other
TCHILGPDCAIEPHDWTKNIXDKIDQIXHDFVX

SEQ ID NO: 17 - protein, 33 aa, synthetic construct
  MOD_RES 22: note = Alpha, Alpha-disubstituted non-natural amino acid with olefinic side chain
  MOD_RES 29: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 22..29: note = The residues are cross-linked to each other
TCHILGPDCAIEPHDWTKNITXKIDQIIXDFVX

SEQ ID NO: 18 - protein, 33 aa, synthetic construct
  MOD_RES 23: note = Alpha, Alpha-disubstituted non-natural amino acid with olefinic side chain
  MOD_RES 30: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 23..30: note = The residues are cross-linked to each other
TCHILGPDCAIEPHDWTKNITDXIDQIIHXFVX

SEQ ID NO: 19 - protein, 33 aa, synthetic construct
  MOD_RES 24: note = Alpha, Alpha-disubstituted non-natural amino acid with olefinic side chain
  MOD_RES 31: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 24..31: note = The residues are cross-linked to each other
TCHILGPDCAIEPHDWTKNITDKXDQIIHDXVX

SEQ ID NO: 20 - protein, 33 aa, synthetic construct
  MOD_RES 25: note = Alpha, Alpha-disubstituted non-natural amino acid with olefinic side chain
  MOD_RES 32: note = Alpha, Alpha-disubstituted non-natural amino acid with olefinic side chain
  MOD_RES 33: note = PEG(n)-cholesterol or PEG(n)-thiocholesterol, wherein n = 1-36
  CROSSLNK 25..32: note = The residues are cross-linked to each other
TCHILGPDCAIEPHDWTKNITDKIXQIIHDFXX

SEQ ID NO: 21 - protein, 33 aa, synthetic construct; identical to SEQ ID NO: 12
  MOD_RES 17: note = (R)-Alpha-(7-prime-octenyl)alanine
  MOD_RES 24: note = (S)-Alpha-(4-prime-pentenyl)alanine
  MOD_RES 33: note = Lys(epsilon-(PEG)4-thiocholesterol)

SEQ ID NO: 22 - protein, 33 aa, synthetic construct; identical to SEQ ID NO: 13
  MOD_RES 18: note = (R)-Alpha-(7-prime-octenyl)alanine
  MOD_RES 25: note = (S)-Alpha-(4-prime-pentenyl)alanine
  MOD_RES 33: note = Lys(epsilon-(PEG)4-thiocholesterol)

SEQ ID NO: 23 - protein, 33 aa, synthetic construct; identical to SEQ ID NO: 14
  MOD_RES 19: note = (R)-Alpha-(7-prime-octenyl)alanine
  MOD_RES 26: note = (S)-Alpha-(4-prime-pentenyl)alanine
  MOD_RES 33: note = Lys(epsilon-(PEG)4-thiocholesterol)

SEQ ID NO: 24 - protein, 33 aa, synthetic construct; identical to SEQ ID NO: 15
  MOD_RES 20: note = (R)-Alpha-(7-prime-octenyl)alanine
  MOD_RES 27: note = (S)-Alpha-(4-prime-pentenyl)alanine
  MOD_RES 33: note = Lys(epsilon-(PEG)4-thiocholesterol)

SEQ ID NO: 25 - protein, 33 aa, synthetic construct; identical to SEQ ID NO: 16
  MOD_RES 21: note = (R)-Alpha-(7-prime-octenyl)alanine
  MOD_RES 28: note = (S)-Alpha-(4-prime-pentenyl)alanine
  MOD_RES 33: note = Lys(epsilon-(PEG)4-thiocholesterol)

SEQ ID NO: 26 - protein, 33 aa, synthetic construct; identical to SEQ ID NO: 17
  MOD_RES 22: note = (R)-Alpha-(7-prime-octenyl)alanine
  MOD_RES 29: note = (S)-Alpha-(4-prime-pentenyl)alanine
  MOD_RES 33: note = Lys(epsilon-(PEG)4-thiocholesterol)

SEQ ID NO: 27 - protein, 33 aa, synthetic construct; identical to SEQ ID NO: 18
  MOD_RES 23: note = (R)-Alpha-(7-prime-octenyl)alanine
  MOD_RES 30: note = (S)-Alpha-(4-prime-pentenyl)alanine
  MOD_RES 33: note = Lys(epsilon-(PEG)4-thiocholesterol)

SEQ ID NO: 28 - protein, 33 aa, synthetic construct; identical to SEQ ID NO: 19
  MOD_RES 24: note = (R)-Alpha-(7-prime-octenyl)alanine
  MOD_RES 31: note = (S)-Alpha-(4-prime-pentenyl)alanine
  MOD_RES 33: note = Lys(epsilon-(PEG)4-thiocholesterol)

SEQ ID NO: 29 - protein, 33 aa, synthetic construct; identical to SEQ ID NO: 20
  MOD_RES 25: note = (R)-Alpha-(7-prime-octenyl)alanine
  MOD_RES 32: note = (S)-Alpha-(4-prime-pentenyl)alanine
  MOD_RES 33: note = Lys(epsilon-(PEG)4-thiocholesterol)

SEQ ID NO: 30 - protein, 32 aa, synthetic construct
  MOD_RES 17: note = Alpha , Alpha-disubstituted non-natural amino acids with olefinic side chain
  MOD_RES 24: note = Alpha, Alpha-disubstituted non-natural amino acids with olefinic side chain
  CROSSLNK 17..24: note = The residues are cross-linked to each other
TCHILGPDCAIEPHDWXKNITDKXDQIIHDFV

SEQ ID NO: 31 - protein, 32 aa, synthetic construct; identical to SEQ ID NO: 6
  MOD_RES 18: note = Alpha, Alpha -disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other

SEQ ID NO: 32 - protein, 32 aa, synthetic construct
  MOD_RES 19: note = Alpha, Alpha-disubstituted non-natural amino acids with olefinic side chain
  MOD_RES 26: note = Alpha, Alpha-disubstituted non-natural amino acids with olefinic side chain
  CROSSLNK 19..26: note = The residues are cross-linked to each other
TCHILGPDCAIEPHDWTKXITDKIDXIIHDFV

SEQ ID NO: 33 - protein, 32 aa, synthetic construct
  MOD_RES 20: note = Alpha, Alpha-disubstituted non-natural amino acids with olefinic side chain
  MOD_RES 27: note = Alpha, Alpha-disubstituted non-natural amino acids with olefinic side chain
  CROSSLNK 20..27: note = The residues are cross-linked to each other
TCHILGPDCAIEPHDWTKNXTDKIDQXIHDFV

SEQ ID NO: 34 - protein, 32 aa, synthetic construct
  MOD_RES 21: note = Alpha, Alpha-disubstituted non-natural amino acids with olefinic side chain
  MOD_RES 28: note = Alpha, Alpha-disubstituted non-natural amino acids with olefinic side chain
  CROSSLNK 21..28: note = The residues are cross-linked to each other
TCHILGPDCAIEPHDWTKNIXDKIDQIXHDFV

SEQ ID NO: 35 - protein, 32 aa, synthetic construct
  MOD_RES 22: note = Alpha, Alpha-disubstituted non-natural amino acids with olefinic side chain
  MOD_RES 29: note = Alpha, Alpha-disubstituted non-natural amino acids with olefinic side chain
  CROSSLNK 22..29: note = The residues are cross-linked to each other
TCHILGPDCAIEPHDWTKNITXKIDQIIXDFV

SEQ ID NO: 36 - protein, 32 aa, synthetic construct
  MOD_RES 23: note = alpha, alpha-disubstituted non-natural amino acids with olefinic side chain
  MOD_RES 30: note = alpha, alpha-disubstituted non-natural amino acids with olefinic side chain
  CROSSLNK 23..30: note = The residues are cross-linked to each other
TCHILGPDCAIEPHDWTKNITDXIDQIIHXFV

SEQ ID NO: 37 - protein, 32 aa, synthetic construct
  MOD_RES 24: note = Alpha, Alpha-disubstituted non-natural amino acids with olefinic side chain
  MOD_RES 31: note = Alpha, Alpha-disubstituted non-natural amino acids with olefinic side chain
  CROSSLNK 24..31: note = The residues are cross-linked to each other
TCHILGPDCAIEPHDWTKNITDKXDQIIHDXV

SEQ ID NO: 38 - protein, 32 aa, synthetic construct
  MOD_RES 25: note = Alpha, Alpha-disubstituted non-natural amino acids with olefinic side chain
  MOD_RES 32: note = Alpha, Alpha-disubstituted non-natural amino acids with olefinic side chain
  CROSSLNK 25..32: note = The residues are cross-linked to each other
TCHILGPDCAIEPHDWTKNITDKIXQIIHDFX

SEQ ID NO: 39 - protein, 32 aa, synthetic construct; identical to SEQ ID NO: 30
  MOD_RES 17: note = (R)-Alpha-(7-prime-octenyl)alanine
  MOD_RES 24: note = (S)-Alpha-(4-prime-pentenyl)alanine

SEQ ID NO: 40 - protein, 32 aa, synthetic construct; identical to SEQ ID NO: 6
  MOD_RES 18: note = (R)-Alpha-(7-prime-octenyl)alanine
  MOD_RES 25: note = (S)-Alpha-(4-prime-pentenyl)alanine

SEQ ID NO: 41 - protein, 32 aa, synthetic construct; identical to SEQ ID NO: 32
  MOD_RES 19: note = (R)-Alpha-(7-prime-octenyl)alanine
  MOD_RES 26: note = (S)-Alpha-(4-prime-pentenyl)alanine

SEQ ID NO: 42 - protein, 32 aa, synthetic construct; identical to SEQ ID NO: 33
  MOD_RES 20: note = (R)-Alpha-(7-prime-octenyl)alanine
  MOD_RES 27: note = (S)-Alpha-(4-prime-pentenyl)alanine

SEQ ID NO: 43 - protein, 32 aa, synthetic construct; identical to SEQ ID NO: 34
  MOD_RES 21: note = (R)-Alpha-(7-prime-octenyl)alanine
  MOD_RES 28: note = (S)-Alpha-(4-prime-pentenyl)alanine

SEQ ID NO: 44 - protein, 32 aa, synthetic construct; identical to SEQ ID NO: 35
  MOD_RES 22: note = (R)-Alpha-(7-prime-octenyl)alanine
  MOD_RES 29: note = (S)-Alpha-(4-prime-pentenyl)alanine

SEQ ID NO: 45 - protein, 32 aa, synthetic construct; identical to SEQ ID NO: 36
  MOD_RES 23: note = (R)-Alpha-(7-prime-octenyl)alanine
  MOD_RES 30: note = (S)-Alpha-(4-prime-pentenyl)alanine

SEQ ID NO: 46 - protein, 32 aa, synthetic construct; identical to SEQ ID NO: 37
  MOD_RES 24: note = (R)-Alpha-(7-prime-octenyl)alanine
  MOD_RES 31: note = (S)-Alpha-(4-prime-pentenyl)alanine

SEQ ID NO: 47 - protein, 32 aa, synthetic construct; identical to SEQ ID NO: 38
  MOD_RES 25: note = (R)-Alpha-(7-prime-octenyl)alanine
  MOD_RES 32: note = (S)-Alpha-(4-prime-pentenyl)alanine

SEQ ID NO: 48 - protein, 31 aa, Sudan ebolavirus
TCRILGPDCCIEPHDWTKNITDKINQIIHDF

SEQ ID NO: 49 - protein, 31 aa, Zaire ebolavirus
TCHILGPDCCIEPHDWTKNITDKIDQIIHDF

SEQ ID NO: 50 - protein, 31 aa, Tai Forest ebolavirus
TCHILGPDCCIEPQDWTKNITDKIDQIIHDF

SEQ ID NO: 51 - protein, 32 aa, Zaire ebolavirus
TCHILGPDCCIEPHDWTKNITDKIDQIIHDFV

SEQ ID NO: 52 - protein, 6 aa, unidentified
  source 1..6: note = Ebolavirus
HHHHHH

SEQ ID NO: 53 - protein, 206 aa, unidentified
  source 1..206: note = Ebolavirus
MGLRQLANETTQALQLFLRATTELRTFSILNRKAIDFLLQRWGGTCHILGPDCAIEPHDWTKNITDKIDQIIHDFGSSGGLRQLANETTQALQLFLRATTELRTFSILNRKAIDFLLQRWGGTCHILGPDCAIEPHDWTKNITDKIDQIIHDFGSSGGLRQLANETTQALQLFLRATTELRTFSILNRKAIDFLLQRWGGHHHHHH

SEQ ID NO: 54 - protein, 16 aa, synthetic construct
TCHILGPDCAIEPHDW

SEQ ID NO: 55 - protein, 6 aa, synthetic construct
KNITDK

SEQ ID NO: 56 - protein, 8 aa, synthetic construct
DQIIHDFV

SEQ ID NO: 57 - protein, 17 aa, synthetic construct
TCHILGPDCAIEPHDWT

SEQ ID NO: 58 - protein, 6 aa, synthetic construct
NITDKI

SEQ ID NO: 59 - protein, 7 aa, synthetic construct
QIIHDFV

SEQ ID NO: 60 - protein, 18 aa, synthetic construct
TCHILGPDCAIEPHDWTK

SEQ ID NO: 61 - protein, 6 aa, synthetic construct
ITDKID

SEQ ID NO: 62 - protein, 6 aa, synthetic construct
IIHDFV

SEQ ID NO: 63 - protein, 19 aa, synthetic construct
TCHILGPDCAIEPHDWTKN

SEQ ID NO: 64 - protein, 6 aa, synthetic construct
TDKIDQ

SEQ ID NO: 65 - protein, 5 aa, synthetic construct
IHDFV

SEQ ID NO: 66 - protein, 20 aa, synthetic construct
TCHILGPDCAIEPHDWTKNI

SEQ ID NO: 67 - protein, 6 aa, synthetic construct
DKIDQI

SEQ ID NO: 68 - protein, 4 aa, synthetic construct
HDFV

SEQ ID NO: 69 - protein, 21 aa, synthetic construct
TCHILGPDCAIEPHDWTKNIT

SEQ ID NO: 70 - protein, 6 aa, synthetic construct
KIDQII

SEQ ID NO: 71 - skipped

SEQ ID NO: 72 - protein, 22 aa, synthetic construct
TCHILGPDCAIEPHDWTKNITD

SEQ ID NO: 73 - protein, 6 aa, synthetic construct
IDQIIH

SEQ ID NO: 74 - skipped

SEQ ID NO: 75 - protein, 23 aa, synthetic construct
TCHILGPDCAIEPHDWTKNITDK

SEQ ID NO: 76 - protein, 6 aa, synthetic construct
DQIIHD

SEQ ID NO: 77 - protein, 24 aa, synthetic construct
TCHILGPDCAIEPHDWTKNITDKI

SEQ ID NO: 78 - protein, 6 aa, synthetic construct
QIIHDF

SEQ ID NO: 79 - protein, 6 aa, synthetic construct
QIIHDF

SEQ ID NO: 80 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDWTXNITDKIXQIIHDFE

SEQ ID NO: 81 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 30: note = diaminobutanoic acid
  CROSSLNK 18..25: note = The residues are cross-linked to each other
TCHILGPDCAIEPHDWTXNITDKIXQIIHXFV

SEQ ID NO: 82 - protein, 32 aa, synthetic construct
  MOD_RES 18: note = Alpha , Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDWTXNITDKIXQIQHDFV

SEQ ID NO: 83 - protein, 32 aa, synthetic construct
  MOD_RES 18: note = Alpha , Alpha-disubstituted non-natural amino acids with olefinic side chain
  MOD_RES 25: note = Alpha, Alpha-disubstituted non-natural amino acids with olefinic side chain
  MOD_RES 26: note = diaminobutanoic acid
  CROSSLNK 18..25: note = The residues are cross-linked to each other
TCHILGPDCAIEPHDWTXNITDKIXXIIHDFV

SEQ ID NO: 84 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDWTXNITDNIXQIIHDFV

SEQ ID NO: 85 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDWTXNILDKIXQIIHDFV

SEQ ID NO: 86 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDWTXEITDKIXQIIHDFV

SEQ ID NO: 87 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDLTXNITDKIXQIIHDFV

SEQ ID NO: 88 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCAIEPHDETXNITDKIXQIIHDFV

SEQ ID NO: 89 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHILGPDCASEPHDWTXNITDKIXQIIHDFV

SEQ ID NO: 90 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHIFGPDCAIEPHDWTXNITDKIXQIIHDFV

SEQ ID NO: 91 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
TCHFLGPDCAIEPHDWTXNITDKIXQIIHDFV

SEQ ID NO: 92 - protein, 32 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  CROSSLNK 18..25: note = The residues are cross-linked to each other
LCHILGPDCAIEPHDWTXNITDKIXQIIHDFV

SEQ ID NO: 93 - protein, 32 aa, synthetic construct; identical to SEQ ID NO: 80
  MOD_RES 18: note = (R)-Alpha-(7-prime-octenyl)alanine
  MOD_RES 25: note = (S)-Alpha-(4-prime-pentenyl)alanine

SEQ ID NO: 94 - protein, 32 aa, synthetic construct; identical to SEQ ID NO: 81
  MOD_RES 18: note = (R)-Alpha-(7-prime-octenyl)alanine
  MOD_RES 25: note = (S)-Alpha-(4-prime-pentenyl)alanine
  MOD_RES 30: note = diaminobutanoic acid

SEQ ID NO: 95 - protein, 32 aa, synthetic construct; identical to SEQ ID NO: 82
  MOD_RES 18: note = (R)-Alpha-(7-prime-octenyl)alanine
  MOD_RES 25: note = (S)-Alpha-(4-prime-pentenyl)alanine

SEQ ID NO: 96 - protein, 32 aa, synthetic construct; identical to SEQ ID NO: 83
  MOD_RES 18: note = (R)-Alpha-(7-prime-octenyl)alanine
  MOD_RES 25: note = (S)-Alpha-(4-prime-pentenyl)alanine
  MOD_RES 26: note = diaminobutanoic acid

SEQ ID NO: 97 - protein, 32 aa, synthetic construct; identical to SEQ ID NO: 84
  MOD_RES 18: note = (R)-Alpha-(7-prime-octenyl)alanine
  MOD_RES 25: note = (S)-Alpha-(4-prime-pentenyl)alanine

SEQ ID NO: 98 - protein, 32 aa, synthetic construct; identical to SEQ ID NO: 85
  MOD_RES 18: note = (R)-Alpha-(7-prime-octenyl)alanine
  MOD_RES 25: note = (S)-Alpha-(4-prime-pentenyl)alanine

SEQ ID NO: 99 - protein, 32 aa, synthetic construct; identical to SEQ ID NO: 86
  MOD_RES 18: note = (R)-Alpha-(7-prime-octenyl)alanine
  MOD_RES 25: note = (S)-Alpha-(4-prime-pentenyl)alanine

SEQ ID NO: 100 - protein, 32 aa, synthetic construct; identical to SEQ ID NO: 87
  MOD_RES 18: note = (R)-Alpha-(7-prime-octenyl)alanine
  MOD_RES 25: note = (S)-Alpha-(4-prime-pentenyl)alanine

SEQ ID NO: 101 - protein, 32 aa, synthetic construct; identical to SEQ ID NO: 88
  MOD_RES 18: note = (R)-Alpha-(7-prime-octenyl)alanine
  MOD_RES 25: note = (S)-Alpha-(4-prime-pentenyl)alanine

SEQ ID NO: 102 - protein, 32 aa, synthetic construct; identical to SEQ ID NO: 89
  MOD_RES 18: note = (R)-Alpha-(7-prime-octenyl)alanine
  MOD_RES 25: note = (S)-Alpha-(4-prime-pentenyl)alanine

SEQ ID NO: 103 - protein, 32 aa, synthetic construct; identical to SEQ ID NO: 90
  MOD_RES 18: note = (R)-Alpha-(7-prime-octenyl)alanine
  MOD_RES 25: note = (S)-Alpha-(4-prime-pentenyl)alanine

SEQ ID NO: 104 - protein, 32 aa, synthetic construct; identical to SEQ ID NO: 91
  MOD_RES 18: note = (R)-Alpha-(7-prime-octenyl)alanine
  MOD_RES 25: note = (S)-Alpha-(4-prime-pentenyl)alanine

SEQ ID NO: 105 - protein, 32 aa, synthetic construct; identical to SEQ ID NO: 92
  MOD_RES 18: note = (R)-Alpha-(7-prime-octenyl)alanine
  MOD_RES 25: note = (S)-Alpha-(4-prime-pentenyl)alanine

SEQ ID NO: 106 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDKIXQIIHDFEX

SEQ ID NO: 107 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 30: note = diaminobutanoic acid
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDKIXQIIHXFVX

SEQ ID NO: 108 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDKIXQIQHDFVX

SEQ ID NO: 109 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 26: note = diaminobutanoic acid
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDKIXXIIHDFVX

SEQ ID NO: 110 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNITDNIXQIIHDFVX

SEQ ID NO: 111 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXNILDKIXQIIHDFVX

SEQ ID NO: 112 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDWTXEITDKIXQIIHDFVX

SEQ ID NO: 113 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDLTXNITDKIXQIIHDFVX

SEQ ID NO: 114 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCAIEPHDETXNITDKIXQIIHDFVX

SEQ ID NO: 115 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHILGPDCASEPHDWTXNITDKIXQIIHDFVX

SEQ ID NO: 116 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHIFGPDCAIEPHDWTXNITDKIXQIIHDFVX

SEQ ID NO: 117 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
TCHFLGPDCAIEPHDWTXNITDKIXQIIHDFVX

SEQ ID NO: 118 - protein, 33 aa, synthetic construct
  MOD_RES 18: note = Alpha, Alpha-disubstituted non-natural amino acids with olefinic side chain
  MOD_RES 25: note = Alpha, Alpha-disubstituted non-natural amino acids with olefinic side chain
  MOD_RES 33: note = PEG(n)-cholesterol or PEG(n)-thiocholesterol, wherein n = 1-36
  CROSSLNK 18..25: note = The residues are cross-linked to each other
LCHILGPDCAIEPHDWTXNITDKIXQIIHDFVX

SEQ ID NO: 119 - protein, 33 aa, synthetic construct; identical to SEQ ID NO: 106
  MOD_RES 18: note = (R)-alpha-(7-prime-octenyl)alanine
  MOD_RES 25: note = (S)-Alpha-(4-prime-pentenyl)alanine
  MOD_RES 33: note = Lys(epsilon-(PEG)4-thiocholesterol

SEQ ID NO: 120 - protein, 33 aa, synthetic construct; identical to SEQ ID NO: 107
  MOD_RES 18: note = (R)-alpha-(7-prime-octenyl)alanine
  MOD_RES 25: note = (S)-Alpha-(4-prime-pentenyl)alanine
  MOD_RES 30: note = diaminobutanoic acid
  MOD_RES 33: note = Lys(epsilon-(PEG)4-thiocholesterol

SEQ ID NO: 121 - protein, 33 aa, synthetic construct; identical to SEQ ID NO: 108
  MOD_RES 18: note = (R)-alpha-(7-prime-octenyl)alanine
  MOD_RES 25: note = (S)-Alpha-(4-prime-pentenyl)alanine
  MOD_RES 33: note = Lys(epsilon-(PEG)4-thiocholesterol

SEQ ID NO: 122 - protein, 33 aa, synthetic construct; identical to SEQ ID NO: 109
  MOD_RES 18: note = (R)-alpha-(7-prime-octenyl)alanine
  MOD_RES 25: note = (S)-Alpha-(4-prime-pentenyl)alanine
  MOD_RES 26: note = diaminobutanoic acid
  MOD_RES 33: note = Lys(epsilon-(PEG)4-thiocholesterol

SEQ ID NO: 123 - protein, 33 aa, synthetic construct; identical to SEQ ID NO: 110
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 124 - protein, 33 aa, synthetic construct; identical to SEQ ID NO: 111
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 125 - protein, 33 aa, synthetic construct; identical to SEQ ID NO: 112
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 126 - protein, 33 aa, synthetic construct; identical to SEQ ID NO: 113
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 127 - protein, 33 aa, synthetic construct; identical to SEQ ID NO: 114
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 128 - protein, 33 aa, synthetic construct; identical to SEQ ID NO: 115
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 129 - protein, 33 aa, synthetic construct; identical to SEQ ID NO: 116
  MOD_RES 18: note = (R)-alpha-(7-prime-octenyl)alanine
  MOD_RES 25: note = (S)-Alpha-(4-prime-pentenyl)alanine
  MOD_RES 33: note = Lys (epsilon-(PEG)4-thiocholesterol)

SEQ ID NO: 130 - protein, 33 aa, synthetic construct; identical to SEQ ID NO: 117
  MOD_RES 18: note = (R)-alpha-(7-prime-octenyl)alanine
  MOD_RES 25: note = (S)-Alpha-(4-prime-pentenyl)alanine
  MOD_RES 33: note = Lys(epsilon-(PEG)4-thiocholesterol

SEQ ID NO: 131 - protein, 33 aa, synthetic construct; identical to SEQ ID NO: 118
  MOD_RES 18: note = (R)-alpha-(7-prime-octenyl)alanine
  MOD_RES 25: note = (S)-Alpha-(4-prime-pentenyl)alanine
  MOD_RES 33: note = Lys(epsilon-(PEG)4-thiocholesterol

SEQ ID NO: 132 - protein, 7 aa, synthetic construct
QIIHDFE

SEQ ID NO: 133 - protein, 7 aa, synthetic construct
  MOD_RES 5: note = diaminobutanoic acid
QIIHXFV

SEQ ID NO: 134 - protein, 7 aa, synthetic construct
QIQHDFV

SEQ ID NO: 135 - protein, 7 aa, synthetic construct
  MOD_RES 1: note = diaminobutanoic acid
XIIHDFV

SEQ ID NO: 136 - protein, 6 aa, synthetic construct
NITDNI

SEQ ID NO: 137 - protein, 6 aa, synthetic construct
NILDKI

SEQ ID NO: 138 - protein, 6 aa, synthetic construct
EITDKI

SEQ ID NO: 139 - protein, 17 aa, synthetic construct
TCHILGPDCAIEPHDLT

SEQ ID NO: 140 - protein, 17 aa, synthetic construct
TCHILGPDCAIEPHDET

SEQ ID NO: 141 - protein, 17 aa, synthetic construct
TCHILGPDCASEPHDWT

SEQ ID NO: 142 - protein, 17 aa, synthetic construct
TCHIFGPDCAIEPHDWT

SEQ ID NO: 143 - protein, 17 aa, synthetic construct
TCHFLGPDCAIEPHDWT

SEQ ID NO: 144 - protein, 17 aa, synthetic construct
LCHILGPDCAIEPHDWT

SEQ ID NO: 145 - protein, 20 aa, synthetic construct
  MOD_RES 20: note = Lys (epsilon-(PEG)4-thiocholesterol)
HDWTKNITDKIDQIIHDFVX

SEQ ID NO: 146 - protein, 20 aa, synthetic construct
  MOD_RES 5: note = R8 octenyl alanine
  MOD_RES 20: note = Lys(epsilon-(PEG)4-thiocholesterol)
  MOD_RES 12: note = S5 pentenyl alanine
HDWTXNITDKIXQIIHDFVX

SEQ ID NO: 147 - protein, 34 aa, unidentified
  source 1..34: note = Ebolavirus
TCHILGPDCCIEPHDWTKNITDKIDQIIHDFVDK

SEQ ID NO: 148 - protein, 33 aa, unidentified
  source 1..33: note = Marburgvirus
TCKVLGPDCCIGIEDLSKNISEQIDQIKKDEQK

SEQ ID NO: 149 - protein, 18 aa, synthetic construct
TCLGPCCIDKNIIDQIDK

SEQ ID NO: 150 - protein, 34 aa, Bombali ebolavirus
TCRILGPDCCIEPHDWTKNITDRIDQIIHDFVDK

SEQ ID NO: 151 - protein, 32 aa, synthetic construct
TCILGPDCCIEPHDWTKNITDIDQIIHDFVDK

SEQ ID NO: 152 - protein, 34 aa, unidentified; identical to SEQ ID NO: 147
  source 1..34: note = Ebolavirus

SEQ ID NO: 153 - protein, 34 aa, synthetic construct
TCRVLGPECCIGVEDLTKNITDQIQQMRRDEQDE

SEQ ID NO: 154 - protein, 19 aa, synthetic construct
TCLGPCCIDTKNITDIQDD

SEQ ID NO: 155 - protein, 34 aa, unidentified; identical to SEQ ID NO: 147
  source 1..34: note = Ebolavirus

SEQ ID NO: 156 - protein, 33 aa, unidentified
  source 1..33: note = Ebolavirus
TCHILGPDCAIEPHDWTKNITDKIDQIIHDFVD

(19) United States
(12) Patent Application Publication
(10) Pub. No.: US 2025/0288658 A1
(43) Pub. Date: Sep. 18, 2025

(54) EBOLAVIRUS SURFACE GLYCOPROTEIN PEPTIDES, CONJUGATES, AND USES THEREOF

(71) Applicant: Dana-Farber Cancer Institute, Inc., Boston, MA (US)

(72) Inventors: Gregory H. Bird, Pelham, NH (US); Loren D. Walensky, Newton, MA (US)

(21) Appl. No.: 18/862,216

(22) PCT Filed: May 3, 2023
(86) PCT No.: PCT/US2023/066545

§ 371 Date: Nov. 1, 2024

Related U.S. Application Data
(60) Provisional application No. 63/364,156, filed on May 4, 2022.

(51) Int. Cl. A61K 39/12; A61K 47/54; A61K 47/60; A61P 31/14; C07K 14/005; C12N 7/00

(52) U.S. Cl. CPC A61K 39/12; A61K 47/543; A61K 47/60; A61P 31/14; C07K 14/005; C12N 7/00; C12N 2760/14122; C12N 2760/14133

(57) ABSTRACT

This disclosure relates to structurally-stabilized (e.g., stapled, e.g., hydrocarbon stapled) Ebolavirus peptides and variants thereof, and structurally-stabilized (e.g., stapled, e.g., hydrocarbon stapled) Ebolavirus peptides and variants thereof, conjugated with polyethylene glycol (PEG) and/or cholesterol (or a variant thereof, e.g., thiocholesterol), e.g., a generated PEG(n)-cholesterol or PEG(n)-thiocholesterol derivatization to further optimize activity, and methods for using such structurally-stabilized peptide conjugates in the prevention and treatment of an Ebolavirus infection or disease in a subject (e.g., human, non-human primate, or fruit bat). The disclosure also relates to methods of using such structurally-stabilized peptides and conjugates in the prevention and treatment of a Marburg virus infection, a Bombali ebolavirus infection, or a Mengla dianlovirus infection in a subject.

### CROSS-REFERENCE TO RELATED APPLICATION

This application claims the priority benefit of U.S. Provisional Application No. 63/364,156, filed May 4, 2022, the content of which is incorporated by reference in its entirety herein.

### SEQUENCE LISTING

The instant application contains a Sequence Listing which has been submitted electronically in XML file format and is hereby incorporated by reference in its entirety. Said XML copy, created on May 3, 2023, is named 00530-0416WO1 and is 257,927 bytes in size.

### TECHNICAL FIELD

This disclosure relates to structurally-stabilized (e.g., stapled, e.g., hydrocarbon stapled) Ebolavirus peptides and structurally-stabilized (e.g., stapled, e.g., hydrocarbon stapled) Ebolavirus peptides conjugated with polyethylene glycol (PEG) and/or cholesterol (or a variant thereof, e.g., thiocholesterol), e.g., a generated PEG(n)-cholesterol or PEG(n)-thiocholesterol derivatization to further optimize activity and methods for using such structurally-stabilized peptide conjugates in the prevention and treatment of an Ebolavirus infection or disease in a subject (e.g., human, non-human primate, or fruit bat). The disclosure also relates to methods of using such structurally-stabilized peptides and conjugates in the prevention and treatment of a Marburg virus infection, a Bombali ebolavirus infection, or a Mengla dianlovirus infection in a subject (e.g., human, non-human primate, or fruit bat).

### BACKGROUND

Ebolaviruses (EBOV) are membrane-enveloped, negative-stranded RNA viruses of the filoviridae family. Within this genus, there are four species that are known to infect humans: Zaire ebolavirus, Bundibugyo ebolavirus, Sudan ebolavirus, and Tai Forest ebolavirus.

EBOV requires fusion of the host and virus membranes to allow for delivery of viral genetic material into the host cell. Membrane fusion in Ebola occurs in host endosomal compartments rather than on the cell surface. Upon engagement of the viral surface glycoprotein (GP1,2) with the cell, the EBOV particle is endocytosed, and its GP is enzymatically cleaved, removing the majority of the GP1 subunit and exposing the transmembrane-anchored subunit GP2. GP2 contains an N-terminal and a C-terminal helical heptad repeat (NHR and CHR, respectively). At its N-terminus, GP2 contains a fusion loop, which, upon a conformational transition, can embed into the host endosomal membrane, leading to a transient intermediate known as the “prehairpin” intermediate in which NHR and CHR are exposed and link the viral and host membranes. Upon pH-mediated maturation of the endosome, GP2 collapses into a highly stable six-helix bundle that brings the host and viral membranes into proximity, providing the driving force for membrane fusion, pore formation, and subsequent infection. The six-helix bundle contains a long, central NHR core with three shorter CHR segments packed alongside in an anti-parallel configuration, together forming a trimeric coiled-coil. An additional intramolecular disulfide bond stabilizes a helix-turn-helix motif between the NHR and CHR and is important for overall bundle stability.

EBOV infections result in severe and often fatal disease (Ebola virus Disease, EVD) in humans. Since its discovery in 1976, the virus has caused several epidemics including in Western Africa (2013-2016) and more recently in the Democratic Republic of Congo (2017-2019). Transmission occurs readily upon direct contact of mucus membranes or non-intact skin with infected body fluids or tissues. EVD is characterized by systemic dissemination of the virus, immune suppression, immune overactivation (cytokine storm), coagulation abnormalities, and tissue damage leading to organ failure and death. In EVD survivors, persistent infection in immune-privileged sites (e.g., central nervous system, eyes, male reproductive tract) occurred. Sexual transmission, male-to-female, has been reported. A number of randomized controlled trials testing preventative vaccines have been completed; however, no approved drugs are currently available for the treatment of Ebola infection. A randomized controlled trial is also underway to test several experimental therapies in addition to supportive care. These experimental therapies or other therapeutic agents are needed to treat the acute disease or RNA persistence following recovery that are easy to administer in an outbreak setting.

Accordingly, there is a need for new inhibitors of Ebola virus for both prevention and treatment.

### SUMMARY

This application relates to compositions and methods disclosing peptide stabilizing technology (e.g., stapling, e.g., hydrocarbon stapling) that recapitulates and fortifies the structure of bioactive helices. In some instances, the peptide stapling is combined with a method for polyethylene glycol (PEG) and/or cholesterol or a cholesterol variant (e.g., thiocholesterol) (e.g., PEG(n)-cholesterol or PEG(n)thiocholesterol) derivatization to generate an optimized and targeted prophylactic and therapeutic agent for prevention and/or treatment of EBOV infection or an EBOV disease. By inserting “staples” (e.g., all-hydrocarbon staples) into EBOV peptides, bioactive-helical structure can be restored and remarkable protease resistance can be conferred by burying the otherwise labile amide bonds at the core of the helical structure and/or restraining amide bonds in a manner that precludes their recognition and proteolysis by the body's proteases. Here, hydrocarbon-stapled and PEG(n)-cholesterol or PEG(n)thiocholesterol derivatized (hydrocarbon-stapled conjugates) peptide inhibitors of EBOV are disclosed. These structurally-stabilized peptides and conjugates are used to prevent and/or treat EBOV infection or EBOV disease.

In some instances, the disclosure herein provides a conjugate comprising a structurally-stabilized peptide (e.g., stapled, e.g., hydrocarbon stapled) and PEG and/or cholesterol or thiocholesterol; wherein the PEG and/or cholesterol or thiocholesterol are linked to the C-terminal amino acid of the structurally-stabilized peptide; wherein the structurally-stabilized peptide comprises:
(a) an amino acid sequence comprising 28-32 contiguous amino acids of the sequence set forth in SEQ ID NO:10 with two to five amino acid substitutions relative to the sequence set forth in SEQ ID NO:10;
   wherein two of the two to five amino acid substitutions are with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32; or
(b) an amino acid sequence comprising 28-32 contiguous amino acids of the sequence set forth in SEQ ID NO:10 with two to five amino acid substitutions relative to the sequence set forth in SEQ ID NO:10 and comprising the formula:

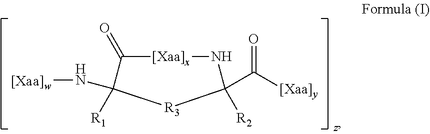

or a pharmaceutically acceptable salt thereof;
   wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
   wherein each R₃ is independently alkane alkylene, alkenylene, or alkynylene, any of which is substituted or unsubstituted;
   wherein z is 1, 2, 3, 4, 5, 6, 7, 8, 9, or 10; and
   wherein:
   (i) each [Xaa]ₓ is KNITDK (SEQ ID NO: 55), or a variant thereof having one amino acid substitution;
   (ii) each [Xaa]ₓ is NITDKI (SEQ ID NO: 58), or a variant thereof having one amino acid substitution;
   (iii) each [Xaa]ₓ is ITDKID (SEQ ID NO: 61), or a variant thereof having one amino acid substitution;
   (iv) each [Xaa]ₓ is TDKIDQ (SEQ ID NO: 64), or a variant thereof having one amino acid substitution;
   (v) each [Xaa]ₓ is DKIDQI (SEQ ID NO: 67), or a variant thereof having one amino acid substitution;
   (vi) each [Xaa]ₓ is KIDQII (SEQ ID NO: 70), or a variant thereof having one amino acid substitution;
   (vii) each [Xaa]ₓ is IDQIIH (SEQ ID NO: 73), or a variant thereof having one amino acid substitution;
   (viii) each [Xaa]ₓ is DQIIHD (SEQ ID NO: 76), or a variant thereof having one amino acid substitution; or
   (ix) each [Xaa]ₓ is QIIHDF (SEQ ID NO: 79), or a variant thereof having one amino acid substitution.

In some instances, the conjugate binds to a 5 helix bundle or fusion bundle intermediate of EBOV GP2 and/or wherein the conjugate inhibits infection of a cell by EBOV in pseudovirus and/or live EBOV virus assay and/or prevents infection of a cell by EBOV in a pseudovirus and/or a live EBOV virus assay. In some instances, the conjugate has one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the conjugate inhibits infection of a cell by EBOV in pseudovirus and/or live EBOV virus assays and/or prevents infection of a cell by EBOV in the pseudovirus and/or the live EBOV virus assays. In some instances, the conjugate comprises the N-terminal non-helical portion of EBOV HR2 (e.g., residues 600-612 of SEQ ID NO:1). In some instances, the conjugate does not comprise amino acids corresponding to positions 634-639 of SEQ ID NO:1. In some instances, the conjugate is 25 to 60 amino acids in length. In some instances, the conjugate is 25 to 40 amino acids in length. In some instances, the conjugate is 28 to 40 amino acids in length. In some instances, the conjugate is 28 to 35 amino acids in length.

In some instances, the conjugate comprises PEG and cholesterol. In some instances, the conjugate comprises PEG(n)-cholesterol, wherein n is 1-36, optionally wherein n is 4, 5, 6, 7, or 8. In some instances, the conjugate comprises Lys(epsilon-PEG(n)-cholesterol, wherein n is 1-36, optionally wherein n is 4, 5, 6, 7, or 8. In some instances, the conjugate comprises Lys(epsilon-(PEG)₄-cholesterol). In some instances, the PEG and cholesterol the PEG and cholesterol comprises the formula:

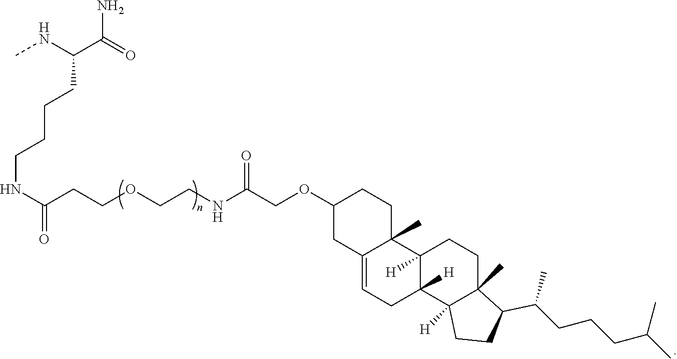

In some instances, the conjugate comprises PEG and thiocholesterol. In some instances, the conjugate comprises PEG(n)-thiocholesterol, wherein n is 2-36, optionally wherein n is 4, 5, 6, 7, or 8. In some instances, the conjugate comprises Lys(epsilon-PEG(n)-thiocholesterol, wherein n is 1-36, optionally wherein n is 4, 5, 6, 7, or 8. In some instances, the conjugate comprises Lys(epsilon-(PEG)₄-thiocholesterol). In some instances, the PEG and thiocholesterol comprises the formula:

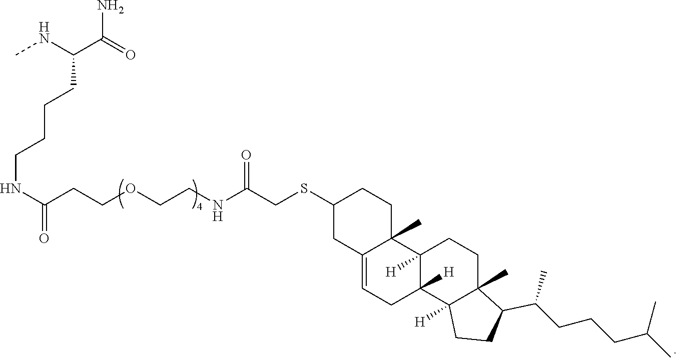

In some instances, the conjugate comprises the amino acid sequence set forth in any one of SEQ ID NOs:30-38. In some instances, the conjugate consists of the amino acid sequence set forth in any one of SEQ ID NOs:30-38. In some instances, the conjugate comprises or consists of the amino acid sequence set forth in SEQ ID NO:31. In some instances, the conjugate comprises or consists of the amino acid sequence set forth in SEQ ID NO:32. In some instances, the conjugate comprises or consists of the amino acid sequence set forth in SEQ ID NO:33. In some instances, the conjugate comprises or consists of the amino acid sequence set forth in SEQ ID NO:34. In some instances, the conjugate comprises or consists of the amino acid sequence set forth in SEQ ID NO:35. In some instances, the conjugate comprises or consists of the amino acid sequence set forth in SEQ ID NO:38.

In some instances, the conjugate comprises the amino acid sequence set forth in any one of SEQ ID NOs: 39-47. In some instances, the conjugate consists of the amino acid sequence set forth in any one of SEQ ID NOs: 39-47. In some instances, the conjugate comprises or consists of the amino acid sequence set forth in SEQ ID NO:40. In some instances, the conjugate comprises or consists of the amino acid sequence set forth in SEQ ID NO:41. In some instances, the conjugate comprises or consists of the amino acid sequence set forth in SEQ ID NO:42. In some instances, the conjugate comprises or consists of the amino acid sequence set forth in SEQ ID NO:43. In some instances, the conjugate comprises or consists of the amino acid sequence set forth in SEQ ID NO:44. In some instances, the conjugate comprises or consists of the amino acid sequence set forth in SEQ ID NO:47.

In some instances, the conjugate comprises the amino acid sequence set forth in any one of SEQ ID NOs: 12-20. In some instances, the conjugate consists of the amino acid sequence set forth in any one of SEQ ID NOs: 12-20. In some instances, the conjugate comprises or consists of the amino acid sequence set forth in SEQ ID NO:13. In some instances, the conjugate comprises or consists of the amino acid sequence set forth in SEQ ID NO:14. In some instances, the conjugate comprises or consists of the amino acid sequence set forth in SEQ ID NO:15. In some instances, the conjugate comprises or consists of the amino acid sequence set forth in SEQ ID NO: 16. In some instances, the conjugate comprises or consists of the amino acid sequence set forth in SEQ ID NO:17. In some instances, the conjugate comprises or consists of the amino acid sequence set forth in SEQ ID NO:20.

In some instances, the conjugate comprises the amino acid sequence set forth in any one of SEQ ID NOs: 21-29. In some instances, the conjugate consists of the amino acid sequence set forth in any one of SEQ ID NOs: 21-29. In some instances, the conjugate comprises or consists of the amino acid sequence set forth in SEQ ID NO:22. In some instances, the conjugate comprises or consists of the amino acid sequence set forth in SEQ ID NO:23. In some instances, the conjugate comprises or consists of the amino acid sequence set forth in SEQ ID NO:24. In some instances, the conjugate comprises or consists of the amino acid sequence set forth in SEQ ID NO:25. In some instances, the conjugate comprises or consists of the amino acid sequence set forth in SEQ ID NO:26. In some instances, the conjugate comprises or consists of the amino acid sequence set forth in SEQ ID NO:29.

In some instances, the conjugate comprises the amino acid sequence set forth in any one of SEQ ID NOs:30-38, except for 1 to 10, 1 to 5, 1 to 3, 2, or 1 amino acid substitutions, insertions, and/or deletions (relative to the amino acid sequence set forth in any one of SEQ ID NOs:30-38), wherein the amino acid substitutions, insertions, and/or deletions are not at the staple position. In some instance, the substitutions are conservative amino acid substitutions. In some instances, the substitutions are on the HR1-interacting face of the conjugate. In some instances, the substitutions are on the HR1-non-interacting face of the conjugate.

In some instances, the conjugate comprises the amino acid sequence set forth in any one of SEQ ID NOs: 39-47, except for 1 to 10, 1 to 5, 1 to 3, 2, or 1 amino acid substitutions, insertions, and/or deletions (relative to the amino acid sequence set forth in any one of SEQ ID NOs:30-38), wherein the amino acid substitutions, insertions, and/or deletions are not at the staple position. In some instance, the substitutions are conservative amino acid substitutions. In some instances, the substitutions are on the HR1-interacting face of the conjugate. In some instances, the substitutions are on the HR1-non-interacting face of the conjugate.

In some instances, the conjugate comprises the amino acid sequence set forth in any one of SEQ ID NOs: 12-20, except for 1 to 10, 1 to 5, 1 to 3, 2, or 1 amino acid substitutions, insertions, and/or deletions (relative to the amino acid sequence set forth in any one of SEQ ID NOs:30-38), wherein the amino acid substitutions, insertions, and/or deletions are not at the staple position. In some instance, the substitutions are conservative amino acid substitutions. In some instances, the substitutions are on the HR1-interacting face of the conjugate. In some instances, the substitutions are on the HR1-non-interacting face of the conjugate.

In some instances, the conjugate comprises the amino acid sequence set forth in any one of SEQ ID NOs: 21-29, except for 1 to 10, 1 to 5, 1 to 3, 2, or 1 amino acid substitutions, insertions, and/or deletions (relative to the amino acid sequence set forth in any one of SEQ ID NOs:30-38), wherein the amino acid substitutions, insertions, and/or deletions are not at the staple position. In some instance, the substitutions are conservative amino acid substitutions. In some instances, the substitutions are on the HR1-interacting face of the conjugate. In some instances, the substitutions are on the HR1-non-interacting face of the conjugate.

In some instances, the conjugate comprises or consists of the sequence of any one of SEQ ID NOs:106-118. In some instances, the conjugate comprises or consists of the sequence of any one of SEQ ID NOs:119-131. In some instances, the conjugate comprises or consists of the sequence of any one of SEQ ID NOs:119, 124, 125, and 127-131. In some instances, the conjugate comprises or consists of the sequence of any one of SEQ ID NOs:106, 111, 112, and 114-118.

Also provided herein is a structurally-stabilized (e.g., stapled, e.g., hydrocarbon stapled) peptide comprising an amino acid sequence comprising 28-32 contiguous amino acids of the sequence set forth in SEQ ID NO:10 with two to five amino acid substitutions relative to the sequence set forth in SEQ ID NO:10; wherein two of the two to five amino acid substitutions are with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32.

In some instances, the structurally-stabilized peptide binds to a 5 helix bundle or fusion bundle intermediate of EBOV GP2 and/or wherein the structurally-stabilized peptide inhibits infection of a cell by EBOV in pseudovirus and/or live EBOV virus assay and/or prevents infection of a cell by EBOV in a pseudovirus and/or a live EBOV virus assay. In some instances, the structurally-stabilized peptide has one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the structurally-stabilized peptide inhibits infection of a cell by EBOV in pseudovirus and/or live EBOV virus assays and/or prevents infection of a cell by EBOV in the pseudovirus and/or the live EBOV virus assays. In some instances, the conjugate comprises the N-terminal non-helical portion of EBOV HR2 (e.g., residues 600-612 of SEQ ID NO:1). In some instances, the structurally-stabilized peptide does not comprise amino acids corresponding to positions 634-639 of SEQ ID NO:1. In some instances, the structurally-stabilized peptide is 25 to 60 amino acids in length. In some instances, the structurally-stabilized peptide is 25 to 40 amino acids in length. In some instances, the structurally-stabilized peptide is 28 to 40 amino acids in length. In some instances, the structurally-stabilized peptide is 28 to 35 amino acids in length.

In some instances, the structurally-stabilized peptide comprises or consists of the amino acid sequence set forth in any one of SEQ ID NOs: 30-38. In some instances, the structurally-stabilized peptide comprises or consists of the amino acid sequence set forth in any one of SEQ ID NOs: 39-47.

In some instances, the structurally-stabilized peptide comprises or consists of the sequence of any one of SEQ ID NOs:80-92. In some instances, the structurally-stabilized peptide comprises or consists of the sequence of any one of SEQ ID NOs:93-105. In some instances, the structurally-stabilized peptide comprises or consists of the sequence of any one of SEQ ID NOs:80, 85, 86, and 88-92. In some instances, the structurally-stabilized peptide comprises or consists of the sequence of any one of SEQ ID NOs:93, 98, 99, and 101-105.

Also provided herein is a structurally-stabilized peptide comprising an amino acid sequence comprising 28-32 contiguous amino acids of the sequence set forth in SEQ ID NO:10 with two to five amino acid substitutions relative to the sequence set forth in SEQ ID NO:10 and comprising the formula:

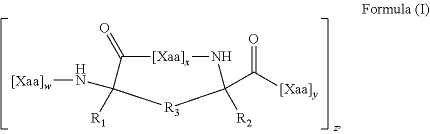

or a pharmaceutically acceptable salt thereof; wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted; wherein each R₃ is independently alkane alkylene, alkenylene, or alkynylene, any of which is substituted or unsubstituted; wherein z is 1, 2, 3, 4, 5, 6, 7, 8, 9, or 10; and wherein:
(i) each [Xaa]ₓ is KNITDK (SEQ ID NO: 55), or a variant thereof having one amino acid substitution;
(ii) each [Xaa]ₓ is NITDKI (SEQ ID NO: 58), or a variant thereof having one amino acid substitution;
(iii) each [Xaa]ₓ is ITDKID (SEQ ID NO: 61), or a variant thereof having one amino acid substitution;
(iv) each [Xaa]ₓ is TDKIDQ (SEQ ID NO: 64), or a variant thereof having one amino acid substitution;
(v) each [Xaa]ₓ is DKIDQI (SEQ ID NO: 67), or a variant thereof having one amino acid substitution;
(vi) each [Xaa]ₓ is KIDQII (SEQ ID NO: 70), or a variant thereof having one amino acid substitution;
(vii) each [Xaa]ₓ is IDQIIH (SEQ ID NO: 73), or a variant thereof having one amino acid substitution;
(viii) each [Xaa]ₓ is DQIIHD (SEQ ID NO: 76), or a variant thereof having one amino acid substitution; or
(ix) each [Xaa]ₓ is QIIHDF (SEQ ID NO: 79), or a variant thereof having one amino acid substitution.

In some instances, the structurally-stabilized peptide binds to a 5 helix bundle or fusion bundle intermediate of EBOV GP2 and/or wherein the structurally-stabilized peptide inhibits infection of a cell by EBOV in pseudovirus and/or live EBOV virus assay and/or prevents infection of a cell by EBOV in a pseudovirus and/or a live EBOV virus assay. In some instances, the structurally-stabilized peptide has one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the structurally-stabilized peptide inhibits infection of a cell by EBOV in pseudovirus and/or live EBOV virus assays and/or prevents infection of a cell by EBOV in the pseudovirus and/or the live EBOV virus assays. In some instances, the structurally-stabilized peptide comprises the N-terminal non-helical portion of EBOV HR2 (e.g., residues 600-612 of SEQ ID NO:1). In some instances, the structurally-stabilized peptide does not comprise amino acids corresponding to positions 634-639 of SEQ ID NO:1. In some instances, the structurally-stabilized peptide is 25 to 60 amino acids in length. In some instances, the structurally-stabilized peptide is 25 to 40 amino acids in length. In some instances, the structurally-stabilized peptide is 28 to 40 amino acids in length. In some instances, the structurally-stabilized peptide is 28 to 35 amino acids in length.

In some instances:
(i) each [Xaa]_{w} is TCHILGPDCAIEPHDW (SEQ ID NO:54), [Xaa]ₓ is KNITDK (SEQ ID NO: 55), and each [Xaa]_{y} is DQIIHDFV (SEQ ID NO:56);
(ii) each [Xaa]_{w} is TCHILGPDCAIEPHDWT (SEQ ID NO:57), [Xaa]ₓ is NITDKI (SEQ ID NO: 58), and each [Xaa]_{y} is QIIHDFV (SEQ ID NO:59);
(iii) each [Xaa]_{w} is TCHILGPDCAIEPHDWTK (SEQ ID NO:60), [Xaa]ₓ is ITDKID (SEQ ID NO: 61), and each [Xaa]_{y} is IIHDFV (SEQ ID NO:62);
(iv) each [Xaa]_{w} is TCHILGPDCAIEPHDWTKN (SEQ ID NO:63), [Xaa]ₓ is TDKIDQ (SEQ ID NO: 64), and each [Xaa]_{y} is IHDFV (SEQ ID NO:65);
(v) each [Xaa]_{w} is TCHILGPDCAIEPHDWTKNI (SEQ ID NO:66), [Xaa]ₓ is DKIDQI (SEQ ID NO: 67), and each [Xaa]_{y} is HDFV (SEQ ID NO:68);
(vi) each [Xaa]_{w} is TCHILGPDCAIEPHDWTKNIT (SEQ ID NO:69), [Xaa]ₓ is KIDQII (SEQ ID NO: 70), and each [Xaa]_{y} is DFV;
(vii) each [Xaa]_{w} is TCHILGPDCAIEPHDWTKNITD (SEQ ID NO:72), [Xaa]ₓ is IDQIIH (SEQ ID NO: 73), and each [Xaa]_{y} is FV;
(viii) each [Xaa]_{w} is TCHILGPDCAIEPHDWTKNITDK (SEQ ID NO:75), [Xaa]ₓ is DQIIHD (SEQ ID NO: 76), and each [Xaa]_{y} is V; or
(ix) each [Xaa]_{w} is TCHILGPDCAIEPHDWTKNITDKI (SEQ ID NO:78), [Xaa]ₓ is QIIHDF (SEQ ID NO: 79), and each [Xaa]_{y} is absent.

Also provided herein is a pharmaceutical composition comprising a conjugate described herein or a structurally-stabilized peptide described herein, and a pharmaceutically acceptable carrier.

Also provided herein is a method of treating an ebolavirus infection in a subject (e.g., a human, non-human primate, or fruit bat) in need thereof, the method comprising administering to the subject a therapeutically-effective amount of a conjugate described herein or of a structurally-stabilized peptide described herein. In some instances, the subject is a human.

Also provided herein is a method of preventing an ebolavirus infection in a subject (e.g., a human, non-human primate, or fruit bat) in need thereof, the method comprising administering to the subject a therapeutically-effective amount of a conjugate described herein or of a structurally-stabilized peptide described herein. In some instances, the subject is a human.

Also provided herein is a method of making a structurally-stabilized peptide, the method comprising: (a) providing a peptide having an amino acid sequence comprising 28-32 contiguous amino acids of the sequence set forth in SEQ ID NO:10 with two to five amino acid substitutions relative to the sequence set forth in SEQ ID NO:10; wherein two of the two to five amino acid substitutions are with α, α-disubstituted non-natural amino acids with olefinic side chains at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32; and
(b) cross-linking the peptide, and optionally purifying the structurally-stabilized peptide. In some instances, the cross-linking is by a ruthenium catalyzed metathesis reaction. In some instances, the method further comprises derivatizing a resin bound amine of the structurally-stabilized peptide with PEG and/or cholesterol containing a carboxylic acid on a resin. In some instances, the method further comprises formulating the structurally-stabilized peptide as a sterile pharmaceutical composition.

Also provided herein is a method of treating a Marburg virus infection, a Bombali ebolavirus infection, or a Mengla dianlovirus infection in a subject in need thereof, the method comprising administering to the subject a therapeutically-effective amount of a conjugate described herein or of a structurally-stabilized peptide described herein. In some instances, the subject is a human.

Also provided herein is a method of preventing a Marburg virus infection, a Bombali ebolavirus infection, or a Mengla dianlovirus infection in a subject in need thereof, the method comprising administering to the subject a therapeutically-effective amount of a conjugate described herein or of a structurally-stabilized peptide described herein. In some instances, the subject is a human.

In some instances of the foregoing methods, the conjugate comprises or consists of the sequence of any one of SEQ ID NOs:119, 124, 125, and 127-131. In some instances of the foregoing methods, the conjugate comprises or consists of the sequence of any one of SEQ ID NOs:106, 111, 112, and 114-118. In some instances of the foregoing methods, the structurally-stabilized peptide comprises or consists of the sequence of any one of SEQ ID NOs:80, 85, 86, and 88-92. In some instances of the foregoing methods, the structurally-stabilized peptide comprises or consists of the sequence of any one of SEQ ID NOs:93, 98, 99, and 101-105.

### BRIEF DESCRIPTION OF DRAWINGS

FIG. 1 depicts an EBOV membrane fusion mechanism (A) and a mechanism of action of stapled lipopeptide fusion inhibitors of Ebolaviruses (B).

FIG. 2A provides the amino acid sequence of an exemplary GP2 protein (SEQ ID NO: 1) of Zaire Ebolavirus, with the underlined sequence indicating exemplary HR2 sequences for construction of structurally-stabilized EBOV peptides and peptide conjugates.

FIG. 2B provides the amino acid sequences of exemplary Ebolavirus Heptad Repeat 1 and 2 domains. SEQ ID NOs:2-5 from top to bottom, respectively.

FIG. 2C shows the structure of an exemplary Ebolavirus six-helix bundle (a trimer of HR1 and HR2 dimers) that mediates fusion between the viral and host membranes. The black region highlights the exemplary HR2 motif that formed the basis for designing structurally-stabilized EBOV peptides and peptide conjugates.

FIG. 2D shows a helical wheel depiction of the C-terminal alpha-helical portion of the EBOV HR2 domain that is subject to structural-stabilization (e.g., peptide stapling).

FIG. 3 shows a variety of α, α-disubstituted non-natural amino acids with olefinic side chains that can be used to generate hydrocarbon stapled EBOV HR2 peptides bearing staples spanning i, i+3; i, i+4; and i, i+7 positions. Single staple scanning is used to generate a library of singly stapled EBOV HR2 peptides for conjugation to PEG(n)-thiocholesterol or PEG(n)-cholesterol moieties.

FIG. 4 shows a variety of staple compositions in multiply stapled peptides and staple scanning to generate a library of multiply stapled EBOV HR2 peptides for conjugation to PEG(n)-thiocholesterol or PEG(n)-cholesterol moieties.

FIG. 5 shows a variety of staple compositions in tandem stitched peptides to generate a library of stitched EBOV HR2 peptides for conjugation to PEG(n)-thiocholesterol or PEG(n)-cholesterol moieties.

FIG. 6 is an illustration of an exemplary approach to designing, synthesizing, and identifying optimal stapled peptide constructs to target the EBOV fusion apparatus, including the generation of Ala scan, staple scan, and variable N- and C-terminal deletion, addition, and derivatization libraries for conjugation to PEG(n)-thiocholesterol or PEG(n)-cholesterol moieties. Singly and doubly stapled and stitched constructs, including alanine and staple and stitch scans, are used to identify optimal stapled peptides for conjugation to PEG(n)-thiocholesterol or PEG(n)-cholesterol moieties and application in in vitro and in vivo analyses.

FIG. 7 shows exemplary PEG(n)-cholesterol derivatizations of the stapled lipopeptide inhibitors of EBOVs.

FIG. 8 show a series of stapled lipopeptide inhibitor compositions to prevent and treat EBOV infections or EBOV diseases based on the EBOV HR2 domain sequences. 8 is an α, α-disubstituted non-natural amino acid with olefinic side chain (e.g., (R)-α-(7′-octenyl)alanine) cross-linked to X. X is an α, α-disubstituted non-natural amino acid with olefinic side chain (e.g., (S)-α-(4′-pentenyl)alanine) cross-linked to 8. A cysteine to alanine mutation is incorporated at amino acid position 609 (numbered according to SEQ ID NO:1).

FIG. 9 shows a synthetic schema of the steps for on-resin derivatization of the stapled peptide sequence (SEQ ID NO:6) with a PEG-linked thiocholesterol moiety.

FIG. 10 shows the differential antiviral activity of a library of i, i+7 stapled, and C609A mutant, cholesterol conjugates (also referred to herein as “lipopeptides”) of an exemplary HR2 sequence bearing a PEG4-thiocholesterol moiety appended on-resin, with a subset of peptides, namely of SEQ ID NO: 22, 23, 24, 25, 26, and 29, showing dose-responsive anti-viral activity. Peptides from top to bottom: SEQ ID NOs: 21-29, respectively. For each peptide, the dose from top to bottom is 0.1 μM, 0.2 μM, 0.4 μM, 0.8 μM, 1.6 μM, 3.1 μM, 6.3 M, 12.5 μM, and 25 μM, respectively.

FIG. 11A shows the dose-response curve for a lead stapled and C609A mutant EBOV peptide conjugate (lipopeptide) inhibitor of EBOV infection (SEQ ID NO:22).

FIG. 11B shows a helical wheel depiction of positions 14-33 of SEQ ID NO:22 (a lead structurally-stabilized EBOV peptide conjugate), with i, i+7 staple localized to the non-interacting face of the HR2 helix.

FIG. 12 shows that an unstapled lipopeptide of SEQ ID NO: 7 exhibits no anti-EBOV activity, yet structural-stabilization of SEQ ID NO:7 (yielding SEQ ID NO:9) confers anti-EBOV activity. Also of note, an i, i+7 stapled lipopeptide of shortened sequence (SEQ ID NO:8) that excludes the non-helical region of the EBOV HR2 domain is inactive in this live virus assay. Sequences from top to bottom: SEQ ID NOs:7-9, respectively.

FIG. 13 depicts the amino acid sequences of SEQ ID NOs:119-131.

FIG. 14 is a graph depicting the differential antiviral activity of the indicated stapled lipopeptides (SEQ ID NOs: 22, 119-122, and 124-131 from top to bottom, respectively; ! is diaminobutanoic acid; *, 8, and X are as defined in FIG. 13. For each stapled lipopeptide, the dose from top to bottom is 0.02 μM, 0.04 M, 0.04 μM, 0.08 M, 0.16 μM, 0.31 μM, 0.63 μM, 1.25 μM, 2.5 μM, 5 μM, and 10 μM.

FIG. 15A is a graph depicting the dose-response curve stapled lipopeptides against EBOV infection; triangle: SEQ ID NO:22; circle: SEQ IDNO:131.

FIG. 15B is a graph depicting the dose-response curve stapled lipopeptides against EBOV infection; triangle: SEQ ID NO:22; circle: SEQ IDNO:130.

FIG. 15C is a graph depicting the dose-response curve stapled lipopeptides against EBOV infection; triangle: SEQ ID NO:22; circle: SEQ IDNO:129.

FIG. 15D is a graph depicting the dose-response curve stapled lipopeptides against EBOV infection; triangle: SEQ ID NO:22; circle: SEQ IDNO:128.

FIG. 15E is a graph depicting the dose-response curve stapled lipopeptides against EBOV infection; triangle: SEQ ID NO:22; circle: SEQ IDNO:127.

FIG. 15F is a graph depicting the dose-response curve stapled lipopeptides against EBOV infection; triangle: SEQ ID NO:22; circle: SEQ IDNO:125.

FIG. 15G is a graph depicting the dose-response curve stapled lipopeptides against EBOV infection; triangle: SEQ ID NO:22; circle: SEQ IDNO:124.

FIG. 15H is a graph depicting the dose-response curve stapled lipopeptides against EBOV infection; triangle: SEQ ID NO:22; circle: SEQ IDNO:119.

FIG. 16A depicts homology at an HR2 domain of Marburg Virus versus Ebola Virus (Zaire strain). SEQ ID NOs: 147, 149, and 148 from top to bottom, respectively.

FIG. 16B is a graph depicting the antiviral activity of the stapled lipopeptide of SEQ ID NO:22 against Marburg virus (pseudovirus: Integral Molecular RVP-1501, Marburg Uganda 2007; cells: 293T-ACE2; peptides: serial 2-fold dilution starting at 10 M; read-out: 72 h). Vehicle treatment average is shown as a dotted line.

FIG. 16C depicts homology between an HIR2 domain of Bombali ebolavirus and an Ebola Virus (Zaire strain) and also homology between an HR2 domain of Mengla dianlovirus and an Ebola Virus (Zaire strain) (SEQ ID NOs:150-155 from top to bottom, respectively).

### DETAILED DESCRIPTION

The present disclosure is based, inter alia, on structurally-stabilized (e.g., stapled, e.g., hydrocarbon stapled) EBOV peptides and the discovery that they may be lipidated (e.g., with PEG and/or cholesterol (or a variant of cholesterol, e.g., thiocholesterol), e.g., PEG(n)-cholesterol or PEG(n)-thiocholesterol) to selectively bind to one or more EBOV and exhibit antiviral activity against EBOV. Accordingly, the present disclosure provides methods (e.g., approaches to convert cholesterol/thiocholesterol into carboxylic acids for on-resin derivatization) and compositions (e.g., structurally-stabilized EBOV peptides and PEG(n)-cholesterol or PEG(n)-thiocholesterol conjugates) for treating, for developing treatments for, and for preventing infection or disease with one or more EBOVs. Thus, the peptides and compositions disclosed herein can be used to prevent and/or treat an EBOV infection or EBOV disease. The peptides and compositions disclosed herein can also be used to treat and/or prevent a Marburg virus infection, a Bombali ebolavirus infection, or a Mengla dianlovirus infection.

### Ebolavirus Peptides

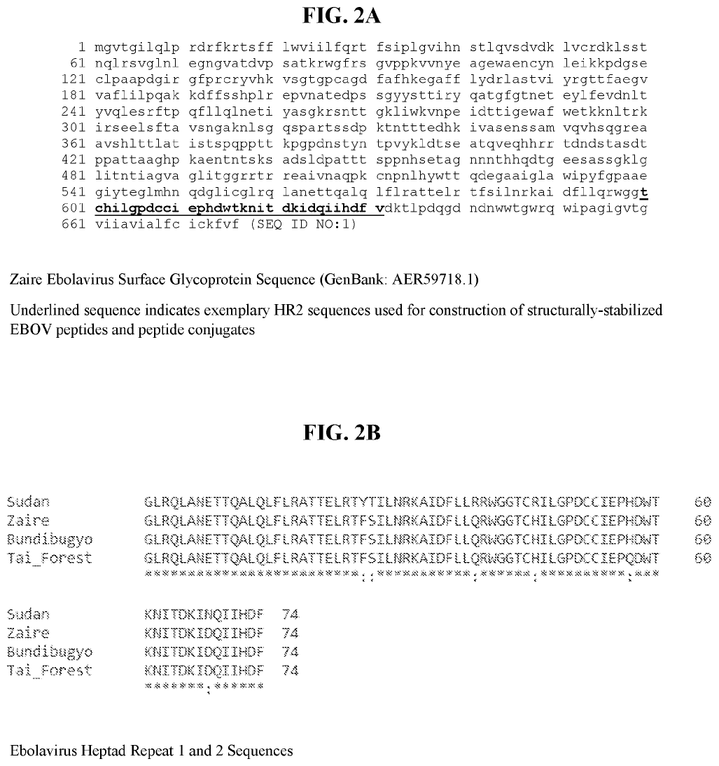

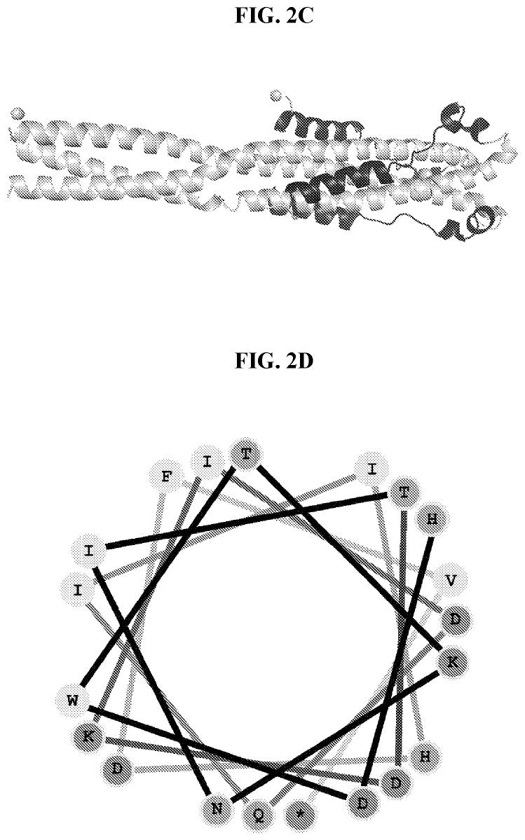

The amino acid sequence (SEQ ID NO:1) of an exemplary Zaire Ebolavirus (EBOV) surface glycoprotein 2 (GP2) sequence is depicted in FIG. 2A.

EBOV GP2 contains an N-terminal helical heptad repeat and a C-terminal helical heptad repeat (NHR and CHR, respectively; also referred to as “HR1” and “HR2”, respectively), separated by a turn/linker. At its N-terminus, GP2 contains a fusion loop, which, upon a conformational transition, can embed into the host endosomal membrane, leading to a transient intermediate known as the “prehairpin” intermediate in which NHR and CHR are exposed and link the viral and host membranes. Upon pH-mediated maturation of the endosome, GP2 collapses into a highly stable six-helix bundle that brings the host and viral membranes into proximity, providing the driving force for membrane fusion, pore formation, and subsequent infection. The six-helix bundle contains a long, central NHR core with three shorter CHR segments packed alongside in an anti-parallel configuration, together forming a trimeric coiled-coil. An additional intramolecular disulfide bond stabilizes a helix-turn-helix motif between the NHR and CHR and is important for overall bundle stability.

The GP2 proteins among the different Ebolavirus species have high homology. See FIG. 2B for an alignment of exemplary amino acid sequences for the HR1 (NHR) and HR2 (CHR) separated by a GG linker for exemplary Sudan ebolavirus, Zaire ebolavirus, Bundibugyo ebolavirus, and Tai Forest ebolavirus GP2 sequences. An exemplary Sudan EBOV HR2 amino acid sequence is TCRILGPDCCIEPHDWTKNITDKINQIIHDF (SEQ ID NO:48). An exemplary Zaire EBOV HR2 amino acid sequence is TCHILGPDCCIEPHDWTKNITDKIDQIIHDF (SEQ ID NO:49). An exemplary Bundibugyo EBOV HR2 amino acid sequence is TCHILGPDCCIEPHDWTKNITDKIDQIIHDF (SEQ ID NO:49). An exemplary Tai Forest EBOV HR2 amino acid sequence is TCHILGPDCCIEPQDWTKNITDKIDQIIHDF (SEQ ID NO:50). In some instances, the EBOV HR2 amino acid sequence further comprises a C-terminal valine corresponding to amino acid position 631 of SEQ ID NO:1 (Val631). For example, in some instances, an exemplary Zaire EBOV HR2 amino acid sequence is TCHILGPDCCIEPHDWTKNITDKIDQIIHDFV (SEQ ID NO:51). In some instances, the EBOV HR2 amino acid sequence further comprises a C-terminal valine corresponding to amino acid position 631 of SEQ ID NO:1 (Val631) and a C-terminal aspartic acid corresponding to amino acid position 632 of SEQ ID NO:1 (Asp632). For example, in some instances, an exemplary EBOV HR2 amino acid sequence is TCHILGPDCAIEPHDWTKNITDKIDQIIHDFVD (SEQ ID NO:156). In some instances, an EBOV HR2 peptide comprises a C609A substitution (numbered according to SEQ ID NO:1. For example, in some instances an EBOV HR2 peptide comprises the amino acid sequence TCHILGPDCAIEPHDWTKNITDKIDQIIHDFV (SEQ ID NO:10).

In certain instances, the EBOV HR2 peptides described herein (e.g., SEQ ID NO: 10) may also contain one or more (e.g., 1, 2, 3, 4, 5, 6, 7, 8, 9, 10, 11, 12, 13, 14, 15, or 16) amino acid substitutions (e.g., relative to the amino acid sequence of SEQ ID NO: 10), e.g., one or more (e.g., 1, 2, 3, 4, 5, 6, 7, 8, 9, 10, 11, 12, 13, 14, 15, or 16) conservative and/or non-conservative amino acid substitutions. In addition, in some instances at least two (e.g., 2, 3, 4, or 5) amino acids (e.g., separated by 2, 3, or 6 amino acids) of an EBOV HR2 peptide described herein (e.g., SEQ ID NO: 10) may be substituted by α, α-disubstituted non-natural amino acids with olefinic side chains that may be cross-linked form one or more staples or stitches. The type of substitutions that are made can, e.g., be guided by an alignment of the HR2 peptide of two or more EBOV GP2 sequences (see, e.g., FIG. 2B). The guidance provided in the Structurally-Stabilized Peptides section below regarding the amino acids that can be varied is equally relevant for the EBOV HR2 peptides described herein. Residues that are unchanged between two or more different EBOV species (e.g., Zaire and Sudan) in such an alignment may be either unmodified or substituted with a non-natural amino acids or conservative amino acids. Residues in the alignment that differ by conservative amino acid substitutions in two or more different EBOV species (e.g., Zaire and Sudan) in such an alignment may be either not replaced or replaced by conservative amino acid substitutions. Residues that are not conserved between two or more different EBOV species (e.g., Zaire and Sudan) in such an alignment may be replaced by any amino acid. In some instances, residues that are conserved between two or more different EBOV species (e.g., Zaire and Sudan) in such an alignment but are located on the non-interacting face of HR2 can be replaced by any amino acid. For example, in view of the alignment in FIG. 2B, conservative amino acid substitutions are permitted at the amino acids corresponding to positions 602, 613, and 624 of SEQ ID NO:1. In certain instances, the substituted amino acid(s) are selected from the group consisting of L-Ala, D-Ala, Aib, Sar, Ser, a substituted alanine, or a substituted glycine derivative.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

In some instances, the EBOV HR2 peptides described herein (e.g., SEQ ID NO: 10) may also contain at least one, at least 2, at least 3, at least 4, or at least 5 (e.g., 1, 2, 3, 4, 5) amino acids added to the N-terminus of the peptide. In some instances, the EBOV HR2 peptides described herein (e.g., SEQ ID NO: 10) may also contain at least one, at least 2, at least 3, at least 4, or at least 5 (e.g., 1, 2, 3, 4, 5) amino acids added to the C-terminus of the peptide. In some instances, the EBOV HR2 peptides described herein (e.g., SEQ ID NO: 10) may also contain at least one, at least 2, at least 3, at least 4, or at least 5 amino acids (e.g., 1, 2, 3, 4, 5) deleted at the N-terminus of the peptide. In some instances, the EBOV HR2 peptides described herein (e.g., SEQ ID NO: 10) may also contain at least one, at least 2, at least 3, at least 4, or at least 5 amino acids (e.g., SEQ ID NO: 10) deleted at the C-terminus of the peptide.

In some cases, the peptides are lipidated. See the Structurally-Stabilized Peptide Conjugates section below. In some cases, the peptides are modified to comprise polyethylene glycol and/or cholesterol (or a cholesterol variant, e.g., thiocholesterol). In some cases, the peptides (e.g., SEQ ID NO: 10) include the following formula affixed to the C-terminus of the peptide:

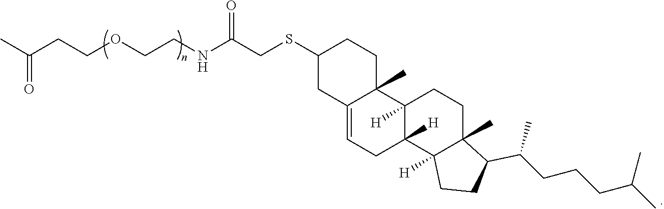

In some instance, the sulfur atom in the formula is replaced with an oxygen atom. In some cases, the peptides (e.g., SEQ ID NO: 10) include the following formula affixed to the C-terminus of the peptide:

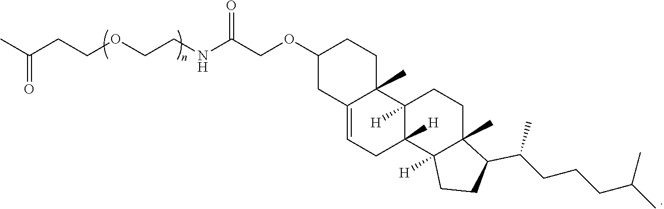

In some instances, n in the above formulas is n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=4. In some instances, n=8.

In some instances, the peptides described herein comprise an amino acid sequence that is at least 50%, at least 60%, at least 70%, at least 80%, at least 85%, or at least 90% identical to sequence set forth in SEQ ID NO:10. In some instances, a peptide as described above (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptides inhibit infection of a cell by EBOV in pseudovirus and/or live EBOV virus assays and/or prevent infection of a cell by EBOV in the pseudovirus and/or the live EBOV virus assays. EBOV pseudovirus assays are known in the art, see, e.g., Steeds et al., 2020, Sci Rep 10, 14289; Li et al., 2018, Rev Med Virol.; 28(1):e1963. doi:10.1002/rmv.1963; Eichler et al., 2021, STAR Protocols, 2(4):100818, ISSN 2666-1667, doi.org/10.1016/j.xpro.2021.100818, each of which is incorporated by reference herein in its entirety.

In some instances, the peptides include an amino acid sequence that has 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, or 2 substitutions, insertions, and/or deletions relative to SEQ ID NO: 10. In some instances, the peptides include 2, 3, 4, 5, or 6 substitutions, insertions, and/or deletions relative to SEQ ID NO: 10. In some instances, a peptide having substitutions, insertions, and/or deletions relative to SEQ ID NO: 10 as described above (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptides inhibit infection of a cell by EBOV in pseudovirus and/or live EBOV virus assays and/or prevent infection of a cell by EBOV in the pseudovirus and/or the live EBOV virus assays.

In some instances, the peptide comprises the N-terminal non-helical portion of EBOV HR2 (e.g., residues 600-612 of SEQ ID NO:1). In some instances, the peptide does not comprise amino acids corresponding to positions 634-639 of SEQ ID NO:1.

In some instances, the peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the peptide is 28 to 40 (e.g., 28, 29, 30, 31, 32, 33, 34, 35, 36, 37, 38, 39, 40) amino acids in length. In some instances, the peptide is 28 to 35 (e.g., 28, 29, 30, 31, 32, 33, 34, 35) amino acids in length. In some instances, the peptide is 32 amino acids in length. In instances in which the peptide is modified to comprise polyethylene glycol and/or cholesterol (or a cholesterol variant, e.g., thiocholesterol), the peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the peptide is 32 amino acids in length.

In some instances, the peptides described above (i) are alpha-helical; (ii) are protease resistant; (iii) bind to a 5 helix bundle or fusion bundle intermediate of EBOV GP2; (iv) inhibit fusion of EBOV with a host cell; and/or (v) inhibit infection of a cell by EBOV. In some instances, the peptides inhibits infection of a cell by EBOV in pseudovirus and/or live EBOV virus assays and/or wherein the structurally-stabilized peptide prevents infection of a cell by EBOV in the pseudovirus and/or the live EBOV virus assays.

In certain instances, each of the EBOV HR2 peptides described above bind to a 5 helix bundle of EBOV GP2 or fusion bundle intermediate of EBOV GP2. In certain instances, each of the EBOV HR2 peptides described above binds to a 5 helix bundle of EBOV GP2 or fusion bundle intermediate of EBOV GP2 and prevents or blocks fusion of an EBOV membrane and a host membrane.

Methods of determining whether a peptide (e.g., a structurally-stabilized peptide described herein) binds to a 5 helix bundle or a fusion bundle intermediate of EBOV GP2 are known in the art, such as, high resolution clear native electrophoresis (hrCNE). See, e.g., Harrison et al., Protein Science, 2011, 20:1587-1596, which is incorporated by reference herein in its entirety. For instance, a tagged (e.g., hexa-histidine (SEQ ID NO:52) tagged) EBOV GP2 ectodomain construct that lacks one of the CHR helices in the post-fusion complex (e.g., a construct having the amino acid sequence MGLRQLANETTQALQLFLRATTELRTFSILNRKAIDFLLQRWGGTCHILGPDCAIE PHDWTKNITDKIDQIIHDFGSSGGLRQLANETTQALQLFLRATTELRTFSILNRKAI DFLLQRWGGTCHILGPDCAIEPHDWTKNITDKIDQIIHDFGSSGGLRQLANETTQA LQLFLRATTELRTFSILNRKAIDFLLQRWGGHHHH-HH (SEQ ID NO:53)) is expressed and incubated with a labeled (e.g., FITC-labeled) control peptide (e.g., a peptide comprising the amino acid sequence of SEQ ID NO:10) or a labeled (e.g., FITC-labeled) test peptide (e.g., a structurally-stabilized peptide described herein) at 37° C. for, e.g., 30 minutes. After incubation, the mixture is analyzed by native PAGE electrophoresis and the native PAGE gel is imaged in a fluorescence scanner to detect the migration of the labeled species and immunoblotted to reveal the location of the tagged EBOV GP2 ectodomain construct. Co-migration of the labeled test peptide and the tagged EBOV GP2 ectodomain construct indicates binding of the test peptide to a 5 helix bundle or a fusion bundle intermediate of EBOV GP2. In some instances, the control peptide is an unstapled version of the test peptide. For example, the control peptide may have the amino acid sequence of the test peptide except that the control peptide contains the corresponding wild type amino acids at the positions of the staple(s) or stitch(es) in the test peptide.

Methods of determining whether a peptide (e.g., a structurally-stabilized peptide or a structurally-stabilized peptide conjugate described herein) prevents or blocks fusion of an EBOV membrane and a host membrane are known in the art, such as, e.g., cytotoxicity and immunofluorescence. In some instances, a peptide prevents or blocks fusion of an Ebola virus membrane and a host membrane if less than 1%, less than 5%, less than 10%, less than 15% less than 20%, less than 30%, less than 40%, or less than 50% of cells are infected with Ebola virus or an EBOV pseudovirus at a multiplicity of infection of 0.1, 0.5, 1, or 10 in the presence the peptide. In some instances, a peptide prevents or blocks fusion of an Ebola virus membrane and a host membrane if less than 1%, less than 5%, less than 10%, less than 15% less than 20%, less than 30%, less than 40%, or less than 50% of cells exhibit fusion of the Ebola virus membrane and the host membrane after infection with Ebola virus at a multiplicity of infection of 0.1, 0.5, 1, or 10 in the presence the peptide.

Methods of determining whether a peptide (e.g., a structurally-stabilized peptide or a structurally-stabilized peptide conjugate described herein) inhibits infection of a cell by EBOV are known in the art, such as, e.g., cytotoxicity and immunofluorescence, and described in the working examples. In some instances, a peptide (e.g., a structurally-stabilized peptide or a structurally-stabilized peptide conjugate described herein) inhibits infection of a cell by EBOV if less than 1%, less than 5%, less than 10%, less than 15% less than 20%, less than 30%, less than 40%, or less than 50% of cells are infected with an EBOV or an EBOV pseudovirus at a multiplicity of infection of 0.1, 0.5, 1, or 10 in the presence the peptide. In some instances, a peptide (e.g., a structurally-stabilized peptide or a structurally-stabilized peptide conjugate described herein) inhibits infection of a cell if the level of EBOV infection of a population of cells in the presence of the peptide is at least 20%, at least 30%, at least 40%, at least 50%, at least 60%, at least 70%, at least 80%, at least 90%, or at least 95% less than the level of EBOV infection of a population of cells in the absence of the peptide under the same conditions. In some instances, the infection with the EBOV is at a multiplicity of infection of 0.1, 0.5, 1, or 10.

### Structurally-Stabilized Peptides

Disclosed herein are structurally-stabilized (e.g., stapled, e.g., hydrocarbon stapled) EBOV peptides based on the HR2 region of the GP2 protein. In some instances, the structurally-stabilized (e.g., stapled, e.g., hydrocarbon stapled) EBOV peptides are derived from EBOV GP2 HR2(600-631, C609A) (TCHILGPDCAIEPHDWTKNITDKIDQIIHDFV (SEQ ID NO:10)). In some instances, the alanine corresponding to residue 10 of SEQ ID NO:10 is substituted with a cysteine as in the native EBOV HR2 sequence (see residue 609 of SEQ ID NO:1).

In some instances, the structurally-stabilized (e.g., stapled, e.g., hydrocarbon stapled) EBOV HR2 peptides comprise or consist of an amino acid sequence comprising 25-32 contiguous amino acids of the sequence set forth in SEQ ID NO:10 with 2 to 16 (e.g., 2, 3, 4, 5, 6, 7, 8, 9, 10, 11, 12, 13, 14, 15, 16) amino acid substitutions relative to the sequence set forth in SEQ ID NO:10; wherein two of the 2 to 16 amino acid substitutions are with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32.

In some instances, the two of the 2 to 16 amino acid substitutions with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 18 and 25,
(ii) positions 19 and 26,
(iii) positions 20 and 27,
(iv) positions 21 and 28,
(v) positions 22 and 29, or
(vi) positions 25 and 32.

In some instances, the two of the 2 to 16 amino acid substitutions with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10): positions 18 and 25.

In some instances, the amino acid sequence comprises 25-32 (e.g., 25, 26, 27, 28, 29, 30, 31, 32) contiguous amino acids of the sequence set forth in SEQ ID NO:10 with 2 to 5 (e.g., 2, 3, 4, 5) amino acid substitutions relative to the sequence set forth in SEQ ID NO:10. In some instances, the amino acid sequence comprises 28-32 (e.g., 28, 29, 30, 31, 32) contiguous amino acids of the sequence set forth in SEQ ID NO:10 with 2 to 16 amino acid substitutions relative to the sequence set forth in SEQ ID NO:10. In some instances, the amino acid sequence comprises 28-32 (e.g., 28, 29, 30, 31, 32) contiguous amino acids of the sequence set forth in SEQ ID NO: 10 with 2 to 5 amino acid substitutions relative to the sequence set forth in SEQ ID NO:10. In certain instances, substitutions to the contiguous amino acid sequence of SEQ ID NO:10 are conservative. In certain instances, substitutions to the contiguous amino acid sequence of SEQ ID NO:10 are non-conservative. Methods for determining the type of substitution are described herein, see, e.g., the Ebolavirus Peptides section above. In some instances, the structurally-stabilized peptide comprises the N-terminal non-helical portion of EBOV HR2 (e.g., residues 600-612 of SEQ ID NO:1). In some instances, the structurally-stabilized peptide does not comprise amino acids corresponding to residues 634-639 of SEQ ID NO:1. In some instances, the structurally-stabilized peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide is 28 to 40 (e.g., 28, 29, 30, 31, 32, 33, 34, 35, 36, 37, 38, 39, 40) amino acids in length. In some instances, the structurally-stabilized peptide is 28 to 35 (e.g., 28, 29, 30, 31, 32, 33, 34, 35) amino acids in length. In some instances, the structurally-stabilized peptide is 32 amino acids in length. In some instances, the structurally-stabilized peptide described above have one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide inhibits infection of a cell by EBOV in pseudovirus and/or live EBOV virus assays and/or prevents infection of a cell by EBOV in the pseudovirus and/or the live EBOV virus assays.

Modifications to an EBOV HR2 peptide (e.g., described herein) can be made by examining the residues at the HR2 binding interface with HR1. The skilled artisan will appreciate that this interface can be discerned, e.g., by looking at the structure of the ebola virus membrane-fusion subunit, gp2, from the envelope glycoprotein ectodomain. See, e.g., Research Collaboratory for Structural Bioinformatics Protein Data Bank (RCSB PDB) ID No. 1EBO (rcsb.org/structure/IEBO).

In some instances, the structurally-stabilized (e.g., stapled, e.g., hydrocarbon stapled) EBOV HR2 peptide is a peptide shown in Table 1, below. In some instances, the structurally-stabilized EBOV HR2 peptide comprises or consists of the amino acid sequence of any one of SEQ ID NOs:31-35 and 38. In some instances, the structurally-stabilized EBOV HR2 peptide comprises or consists of the amino acid sequence of any one of SEQ ID NOs:40-44 and 47.

TABLE 1
Structurally-Stabilized EBOV HR2 Peptides

| | SEQ | | SEQ |
|---|---|---|---|
| | ID | | ID |
| Sequence | NO | Sequence | NO |
| TCHILGPDCAIEPHDW8KNITDKXDQIIHDFV | 30 | TCHILGPDCAIEPHDW8KNITDKXDQIIHDFV | 39 |
| TCHILGPDCAIEPHDWT8NITDKIXQIIHDFV | 31 | TCHILGPDCAIEPHDWT8NITDKIXQIIHDFV | 40 |
| TCHILGPDCAIEPHDWTK8ITDKIDXIIHDFV | 32 | TCHILGPDCAIEPHDWTK8ITDKIDXIIHDFV | 41 |
| TCHILGPDCAIEPHDWTKN8TDKIDQXIHDFV | 33 | TCHILGPDCAIEPHDWTKN8TDKIDQXIHDFV | 42 |
| TCHILGPDCAIEPHDWTKNI8DKIDQIXHDFV | 34 | TCHILGPDCAIEPHDWTKNI8DKIDQIXHDFV | 43 |
| TCHILGPDCAIEPHDWTKNIT8KIDQIIXDFV | 35 | TCHILGPDCAIEPHDWTKNIT8KIDQIIXDFV | 44 |
| TCHILGPDCAIEPHDWTKNITD8IDQIIHXFV | 36 | TCHILGPDCAIEPHDWTKNITD8IDQIIHXFV | 45 |
| TCHILGPDCAIEPHDWTKNITDK8DQIIHDXV | 37 | TCHILGPDCAIEPHDWTKNITDK8DQIIHDXV | 46 |
| TCHILGPDCAIEPHDWTKNITDKI8QIIHDFX | 38 | TCHILGPDCAIEPHDWTKNITDKI8QIIHDFX | 47 |
| TCHILGPDCAIEPHDWT8NITDKIXQIIHDFE | 80 | TCHILGPDCAIEPHDWT8NITDKIXQIIHDFE | 93 |
| TCHILGPDCAIEPHDWT8NITDKIXQIIH#FV | 81 | TCHILGPDCAIEPHDWT8NITDKIXQIIH#FV | 94 |
| TCHILGPDCAIEPHDWT8NITDKIXQIQHDFV | 82 | TCHILGPDCAIEPHDWT8NITDKIXQIQHDFV | 95 |
| TCHILGPDCAIEPHDWT8NITDKIX#IIHDFV | 83 | TCHILGPDCAIEPHDWT8NITDKIX#IIHDFV | 96 |
| TCHILGPDCAIEPHDWT8NITDNIXQIIHDFV | 84 | TCHILGPDCAIEPHDWT8NITDNIXQIIHDFV | 97 |
| TCHILGPDCAIEPHDWT8NILDKIXQIIHDFV | 85 | TCHILGPDCAIEPHDWT8NILDKIXQIIHDFV | 98 |
| TCHILGPDCAIEPHDWT8EITDKIXQIIHDFV | 86 | TCHILGPDCAIEPHDWT8EITDKIXQIIHDFV | 99 |
| TCHILGPDCAIEPHDLT8NITDKIXQIIHDFV | 87 | TCHILGPDCAIEPHDLT8NITDKIXQIIHDFV | 100 |
| TCHILGPDCAIEPHDET8NITDKIXQIIHDFV | 88 | TCHILGPDCAIEPHDET8NITDKIXQIIHDFV | 101 |
| TCHILGPDCASEPHDWT8NITDKIXQIIHDFV | 89 | TCHILGPDCASEPHDWT8NITDKIXQIIHDFV | 102 |
| TCHIFGPDCAIEPHDWT8NITDKIXQIIHDFV | 90 | TCHIFGPDCAIEPHDWT8NITDKIXQIIHDFV | 103 |
| TCHFLGPDCAIEPHDWT8NITDKIXQIIHDFV | 91 | TCHFLGPDCAIEPHDWT8NITDKIXQIIHDFV | 104 |
| LCHILGPDCAIEPHDWT8NITDKIXQIIHDFV | 92 | LCHILGPDCAIEPHDWT8NITDKIXQIIHDFV | 105 |

In Table 1, with respect to SEQ ID NOs: 30-38 and 80-92, “8”=α, α-disubstituted non-natural amino acids with olefinic side chain cross-linked to X; “X”=α, α-disubstituted non-natural amino acids with olefinic side chain cross-linked to 8. In Table 1, with respect to SEQ ID NOs: 39-47 and 93-105, “8”=(R)-α-(7′-octenyl)alanine; “X”=(S)-α-(4′-pentenyl)alanine. In Table 1, # is diaminobutanoic acid.

In some instances, a peptide of Table 1 forms part of a structurally-stabilized peptide conjugate described in the Structurally-Stabilized Peptide Conjugate section below.

Note that the bolded residues in Table 1 identify the stapling amino acids. In some instances (e.g., a peptide described in Table 1), the structurally-stabilized peptide is single-stapled peptide.

The disclosure encompasses each and every peptide and structurally-stabilized peptide listed in Table 1 as well as variants thereof (e.g., having 1, 2, 3, 4, 5, 6, 7, 8, 9, 10, 11, 12, 13, 14, 15, 16) amino acid substitutions, insertions, and/or deletions, except at the positions of the staple (i.e., the bolded residues in Table 1). In some instances, the variant has 1 to 10 amino acid substitutions, insertions, and/or deletions, except at the positions of the staple (i.e., the bolded residues in Table 1). In some instances, the variant has 1 to 5 amino acid substitutions, insertions, and/or deletions, except at the positions of the staple, (i.e., the bolded residues in Table 1). In some instances, the variant has 1 to 3 amino acid substitutions, insertions, and/or deletions, except at the positions of the staple, (i.e., the bolded residues in Table 1). In some instances, the variant has 1 to 10, 10 to 5, 1 to 3, 2, or 1 amino acid substitutions, insertions, and/or deletions, except at the positions of the staple (i.e., the bolded residues in Table 1), relative to the amino acid sequence set forth in any one of SEQ ID NOs: 31-35 and 38. In some instances, the variant has 1 to 10, 10 to 5, 1 to 3, 2, or 1 amino acid substitutions, insertions, and/or deletions, except at the positions of the staple (i.e., the bolded residues in Table 1), relative to the amino acid sequence set forth in any one of SEQ ID NOs: 40-44 and 47. In some instances, the structurally-stabilized peptide comprises the N-terminal non-helical portion of EBOV HR2 (e.g., residues 600-612 of SEQ ID NO:1). In some instances, the structurally-stabilized peptide does not comprise amino acids corresponding to residues 634-639 of SEQ ID NO: 1. In some instances, the structurally-stabilized peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide is 28 to 40 (e.g., 28, 29, 30, 31, 32, 33, 34, 35, 36, 37, 38, 39, 40) amino acids in length. In some instances, the structurally-stabilized peptide is 28 to 35 (e.g., 28, 29, 30, 31, 32, 33, 34, 35) amino acids in length. In some instances, the structurally-stabilized peptide is 32 amino acids in length. In some instances, the structurally-stabilized peptide described above have one or more (1, 2, 3, 4, 5, 6) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide inhibits infection of a cell by EBOV in pseudovirus and/or live EBOV virus assays and/or prevents infection of a cell by EBOV in the pseudovirus and/or the live EBOV virus assays.

In some instances, the structurally-stabilized peptide includes an amino acid sequence that has 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, or 2 substitutions, insertions, and/or deletions relative to SEQ ID NO: 10. In some instances, a structurally-stabilized peptide having substitutions, insertions, and/or deletions relative to SEQ ID NO: 10 as described above (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide inhibits infection of a cell by EBOV in pseudovirus and/or live EBOV virus assays and/or prevents infection of a cell by EBOV in the pseudovirus and/or the live EBOV virus assays.

In some instances, disclosed herein are peptides that comprise 0-16 (0, 1, 2, 3, 4, 5, 6, 7, 8, 9, 10, 11, 12, 13, 14, 15, 16) amino acid substitutions compared to one of the single-stapled peptides in Table 1. In some instances, disclosed herein are peptides that are at least 50%, at least 60%, at least 70%, at least 80%, or at least 90% identical to one of the single-stapled peptides in Table 1. In some instances, disclosed herein are peptides that are at least 50%, at least 60%, at least 70%, at least 80%, or at least 90% identical to the amino acid sequence set forth in any one of SEQ ID NOs: 31-35 and 38. In some instances, disclosed herein are peptides that are at least 50%, at least 60%, at least 70%, at least 80%, or at least 90% identical to the amino acid sequence set forth in any one of SEQ ID NOs: 40-44 and 47. It is understood that the variation is not at the staple position (i.e., the bolded residues in Table 1). In some instances, disclosed herein are peptides that are 100% identical to one of the single-stapled peptides in Table 1. In some instances, disclosed herein are peptides that are 100% identical to the amino acid sequence set forth in any one of SEQ ID NOs: 31-35 and 38. In some instances, disclosed herein are peptides that are 100% identical to one of the single-stapled peptides in Table 1. In some instances, disclosed herein are peptides that are 100% identical to the amino acid sequence set forth in any one of SEQ ID NOs: 40-44 and 47. In some instances, the structurally-stabilized peptide comprises the N-terminal non-helical portion of EBOV HR2 (e.g., residues 600-612 of SEQ ID NO:1). In some instances, the structurally-stabilized peptide does not comprise amino acids corresponding to positions 634-639 of SEQ ID NO:1. In some instances, the structurally-stabilized peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide is 28 to 40 (e.g., 28, 29, 30, 31, 32, 33, 34, 35, 36, 37, 38, 39, 40) amino acids in length. In some instances, the structurally-stabilized peptide is 28 to 35 (e.g., 28, 29, 30, 31, 32, 33, 34, 35) amino acids in length. In some instances, the structurally-stabilized peptide is 32 amino acids in length. In some instances, the structurally-stabilized peptide described above have one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide inhibits infection of a cell by EBOV in pseudovirus and/or live EBOV virus assays and/or prevents infection of a cell by EBOV in the pseudovirus and/or the live EBOV virus assays.

In some instances, any substitution as described herein can be a conservative substitution. In some instances, any substitution as described herein is a non-conservative substitution.

In some instances, a structurally-stabilized peptide described herein comprises an E (or a conservative substitution of an E) at the amino acid corresponding to position 32 of SEQ ID NO:10. In some instances, a structurally-stabilized peptide described herein comprises a diaminobutanoic acid (or a conservative substitution of a diaminobutanoic acid) at the amino acid corresponding to position 30 of SEQ ID NO:10. In some instances, a structurally-stabilized peptide described herein comprises a Q (or a conservative substitution of a Q) at the amino acid corresponding to position 28 of SEQ ID NO:10. In some instances, a structurally-stabilized peptide described herein comprises a diaminobutanoic acid (or a conservative substitution of a diaminobutanoic acid) at the amino acid corresponding to position 26 of SEQ ID NO:10. In some instances, a structurally-stabilized peptide described herein comprises an N (or a conservative substitution of an N) at the amino acid corresponding to position 23 of SEQ ID NO:10. In some instances, a structurally-stabilized peptide described herein comprises an L (or a conservative substitution of an L) at the amino acid corresponding to position 21 of SEQ ID NO:10. In some instances, a structurally-stabilized peptide described herein comprises an E (or a conservative substitution of an E) at the amino acid corresponding to position 19 of SEQ ID NO:10. In some instances, a structurally-stabilized peptide described herein comprises an L (or a conservative substitution of an L) at the amino acid corresponding to position 16 of SEQ ID NO:10. In some instances, a structurally-stabilized peptide described herein comprises an E or an L (or a conservative substitution of an E or an L) at the amino acid corresponding to position 16 of SEQ ID NO:10. In some instances, a structurally-stabilized peptide described herein comprises an S (or a conservative substitution of an S) at the amino acid corresponding to position 11 of SEQ ID NO:10. In some instances, a structurally-stabilized peptide described herein comprises an F (or a conservative substitution of an F) at the amino acid corresponding to position 5 of SEQ ID NO:10. In some instances, a structurally-stabilized peptide described herein comprises an F (or a conservative substitution of an F) at the amino acid corresponding to position 4 of SEQ ID NO:10. In some instances, a structurally-stabilized peptide described herein comprises an L (or a conservative substitution of an L) at the amino acid corresponding to position 1 of SEQ ID NO:10.

It is understood that the variation is not at a mutated position (i.e., the bolded, italicized residues in Table 1) or is with a conservative amino acid substitution at the mutated position.

In some instances, the non-natural amino acids that may be used as stapling amino acids are: (R)-2-(2′-propenyl)alanine; (R)-2-(4′-pentenyl)alanine; (R)-α-(7′-octenyl)alanine; (S)-α-(2′-propenyl)alanine; (S)-α-(4′-pentenyl)alanine; (S)-2-(7′-octenyl)alanine; α,α-Bis(4′-pentenyl)glycine; and α,α-Bis(7′-octeny)glycine.

In some instances, an internal staple replaces the side chains of 2 amino acids, i.e., each staple is between two amino acids separated by, for example, 6 amino acids. In some instances, the amino acids forming the staple are at each of positions i and i+7 of the staple. For example, where a peptide has the sequence . . . X1, X2, X3, X4, X5, X6, X7, X8, X9 . . . , cross-links between X1 and X8 (i and i+7) are useful hydrocarbon stapled forms of that peptide. The use of an i and i+4 staple, multiple cross-links (e.g., 2, 3, 4, or more), or a tandem stitch is also contemplated. Additional description regarding making and use of hydrocarbon-stapled peptides can be found, e.g., in U.S. Patent Publication Nos. 2012/0172285, 2010/0286057, and 2005/0250680, the contents of all of which are incorporated by reference herein in their entireties.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). The structural-stabilization may be by, e.g., stapling the peptide (see, e.g., Walensky, J Med. Chem., 57:6275-6288 (2014), the contents of which are incorporated by reference herein in its entirety). In some cases, the staple is a hydrocarbon staple.

In some instances, a staple used herein is an all hydrocarbon staple.

In some instances, a staple used herein is a lactam staple; a UV-cycloaddition staple; an oxime staple; a thioether staple; a double-click staple; a bis-lactam staple; a bis-arylation staple; or a combination of any two or more thereof. Stabilized peptides as described herein include stapled peptides as well as peptides containing multiple staples or any other chemical strategies for structural reinforcement (see. e.g., Balaram P. Cur. Opin. Struct. Biol. 1992; 2:845; Kemp D S, et al., J Am. Chem. Soc. 1996; 118:4240; Orner B P, et al., J. Am. Chem. Soc. 2001; 123:5382; Chin J W, et al., Jnt. Ed. 2001; 40:3806; Chapman R N, et al., J. Am. Chem. Soc. 2004; 126:12252; Horne W S, et al., Chem., Int. Ed. 2008; 47:2853; Madden et al., Chem Commun (Camb). 2009 Oct. 7; (37): 5588-5590; Lau et al., Chem. Soc. Rev., 2015, 44:91-102; and Gunnoo et al., Org. Biomol. Chem., 2016, 14:8002-8013; each of which is incorporated by reference herein in its entirety).

A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and may increase target binding affinity, hydrophobicity, plasma membrane binding, and/or cell permeability. Accordingly, the stapled (cross-linked) peptides described herein have improved biological activity and pharmacology relative to a corresponding non-stapled (un-cross-linked) peptide.

In some instances, the structurally-stabilized EBOV HR2 peptide comprises an amino acid sequence comprising 25-32 contiguous amino acids of the sequence set forth in SEQ ID NO:10 with 2 to 16 (e.g., 2, 3, 4, 5, 6, 7, 8, 9, 10, 11, 12, 13, 14, 15, 16) amino acid substitutions relative to the sequence set forth in SEQ ID NO:10 and comprising the formula:

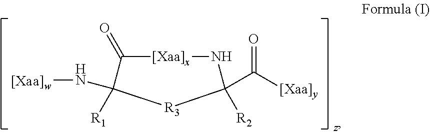

or a pharmaceutically acceptable salt thereof;
wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
wherein each R₃ is independently alkane alkylene, alkenylene, or alkynylene, any of which is substituted or unsubstituted;
wherein z is 1, 2, 3, 4, 5, 6, 7, 8, 9, or 10; and
wherein:
   (i) each [Xaa]ₓ is KNITDK (SEQ ID NO: 55), or a variant thereof having one amino acid substitution;
   (ii) each [Xaa]ₓ is NITDKI (SEQ ID NO: 58), or a variant thereof having one amino acid substitution;
   (iii) each [Xaa]ₓ is ITDKID (SEQ ID NO: 61), or a variant thereof having one amino acid substitution;
   (iv) each [Xaa]ₓ is TDKIDQ (SEQ ID NO: 64), or a variant thereof having one amino acid substitution;
   (v) each [Xaa]ₓ is DKIDQI (SEQ ID NO: 67), or a variant thereof having one amino acid substitution;
   (vi) each [Xaa]ₓ is KIDQII (SEQ ID NO: 70), or a variant thereof having one amino acid substitution;
   (vii) each [Xaa]ₓ is IDQIIH (SEQ ID NO: 73), or a variant thereof having one amino acid substitution;
   (viii) each [Xaa]ₓ is DQIIHD (SEQ ID NO: 76), or a variant thereof having one amino acid substitution; or
   (ix) each [Xaa]ₓ is QIIHDF (SEQ ID NO: 79), or a variant thereof having one amino acid substitution.

In some instances, the amino acid sequence comprises 25-32 (e.g., 25, 26, 27, 28, 29, 30, 31, 32) contiguous amino acids of the sequence set forth in SEQ ID NO:10 with 2 to 5 (e.g., 2, 3, 4, 5) amino acid substitutions relative to the sequence set forth in SEQ ID NO:10. In some instances, the amino acid sequence comprises 28-32 (e.g., 28, 29, 30, 31, 32) contiguous amino acids of the sequence set forth in SEQ ID NO:10 with 2 to 16 amino acid substitutions relative to the sequence set forth in SEQ ID NO:10. In some instances, the amino acid sequence comprises 28-32 (e.g., 28, 29, 30, 31, 32) contiguous amino acids of the sequence set forth in SEQ ID NO:10 with 2 to 5 amino acid substitutions relative to the sequence set forth in SEQ ID NO:10.

In certain instances, substitutions to the contiguous amino acid sequence of SEQ ID NO:10 are conservative. In certain instances, substitutions to the contiguous amino acid sequence of SEQ ID NO:10 are non-conservative. Methods for determining the type of substitution are described herein, see, e.g., the Ebolavirus Peptides section above. In some instances, the structurally-stabilized peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide is 28 to 40 (e.g., 28, 29, 30, 31, 32, 33, 34, 35, 36, 37, 38, 39, 40) amino acids in length. In some instances, the structurally-stabilized peptide is 28 to 35 (e.g., 28, 29, 30, 31, 32, 33, 34, 35) amino acids in length. In some instances, the structurally-stabilized peptide is 32 amino acids in length. In some instances, the structurally-stabilized peptide described above have one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide inhibits infection of a cell by EBOV in pseudovirus and/or live EBOV virus assays and/or prevents infection of a cell by EBOV in the pseudovirus and/or the live EBOV virus assays.

In some instances of Formula (I),
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄-K-R₄]n; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

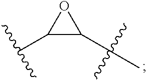 [SMILES: *C1OC1*]

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid.

In some instances, each of the [Xaa]_{w} of Formula (I), the [Xaa]ₓ of Formula (I), and the [Xaa]_{y} of Formula (I) is as described for any one of constructs 1-9 of Table 2. For example, for a structurally-stabilized peptide comprising the [Xaa]_{w}, the [Xaa]ₓ, and the [Xaa]_{y} of construct 1 of Table 2, the [Xaa]_{w}, the [Xaa]ₓ, and the [Xaa]_{y} are: TCHILGPDCAIEPHDW (SEQ ID NO:54), KNITDK (SEQ ID NO: 55), and DQIIDFV (SEQ ID NO:56), respectively.

TABLE 2
[Xaa]_{w}, [Xaa]ₓ, and [Xaa]_{y} sequences for Formula (I) constructs 1-
15. In Table 2, # is diaminobutanoic acid.

| Construct | [Xaa]_{w} | [Xaa]ₓ | [Xaa]_{y} |
|---|---|---|---|
| 1 | TCHILGPDCAIEPHDW | KNITDK (SEQ ID NO: | DQIIHDFV (SEQ ID |
| | (SEQ ID NO: 54) | 55) | NO: 56) |
| 2 | TCHILGPDCAIEPHDW | NITDKI (SEQ ID NO: | QIIHDFV (SEQ ID |
| | T (SEQ ID NO: 57) | 58) | NO: 59) |
| 3 | TCHILGPDCAIEPHDW | ITDKID (SEQ ID NO: | IIHDFV (SEQ ID |
| | TK (SEQ ID NO: 60) | 61) | NO: 62) |
| 4 | TCHILGPDCAIEPHDW | TDKIDQ (SEQ ID NO: | IHDFV (SEQ ID |
| | TKN (SEQ ID NO: 63) | 64) | NO: 65) |
| 5 | TCHILGPDCAIEPHDW | DKIDQI (SEQ ID NO: | HDFV (SEQ ID NO: 68) |
| | TKNI (SEQ ID NO: 66) | 67) | |
| 6 | TCHILGPDCAIEPHDW | KIDQII (SEQ ID NO: | DFV |
| | TKNIT (SEQ ID NO: 69) | 70) | |
| 7 | TCHILGPDCAIEPHDW | IDQIIH (SEQ ID NO: | FV |
| | TKNITD (SEQ ID | 73) | |
| | NO: 72) | | |
| 8 | TCHILGPDCAIEPHDW | DQIIHD (SEQ ID NO: | V |
| | TKNITDK (SEQ ID | 76) | |
| | NO: 75) | | |
| 9 | TCHILGPDCAIEPHDW | QIIHDF (SEQ ID NO: | Absent |
| | TKNITDKI (SEQ ID | 78) | |
| | NO: 77) | | |
| 10 | TCHILGPDCAIEPHDW | NITDKI (SEQ ID NO: | QIIHDFE (SEQ ID NO: |
| | T (SEQ ID NO: 57) | 58) | 132) |
| 11 | TCHILGPDCAIEPHDW | NITDKI (SEQ ID NO: | QIIH#FV (SEQ ID NO: |
| | T (SEQ ID NO: 57) | 58) | 133) |
| 12 | TCHILGPDCAIEPHDW | NITDKI (SEQ ID NO: | QIQHDFV (SEQ ID |
| | T (SEQ ID NO: 57) | 58) | NO: 134) |
| 13 | TCHILGPDCAIEPHDW | NITDKI (SEQ ID NO: | #IIHDFV (SEQ ID NO: |
| | T (SEQ ID NO: 57) | 58) | 135) |
| 14 | TCHILGPDCAIEPHDW | NITDNI (SEQ ID NO: | QIIHDFV (SEQ ID |
| | T (SEQ ID NO: 57) | 136) | NO: 59) |
| 15 | TCHILGPDCAIEPHDW | NILDKI (SEQ ID NO: | QIIHDFV (SEQ ID |
| | T (SEQ ID NO: 57) | 137) | NO: 59) |
| 16 | TCHILGPDCAIEPHDW | EITDKI (SEQ ID NO: | QIIHDFV (SEQ ID |
| | T (SEQ ID NO: 57) | 138) | NO: 59) |
| 17 | TCHILGPDCAIEPHDL | NITDKI (SEQ ID NO: | QIIHDFV (SEQ ID |
| | T (SEQ ID NO: 139) | 58) | NO: 59) |
| 18 | TCHILGPDCAIEPHDE | NITDKI (SEQ ID NO: | QIIHDFV (SEQ ID |
| | T (SEQ ID NO: 140) | 58) | NO: 59) |
| 19 | TCHILGPDCASEPHD | NITDKI (SEQ ID NO: | QIIHDFV (SEQ ID |
| | WT (SEQ ID NO: 141) | 58) | NO: 59) |
| 20 | TCHIFGPDCAIEPHDW | NITDKI (SEQ ID NO: | QIIHDFV (SEQ ID |
| | T (SEQ ID NO: 142) | 58) | NO: 59) |
| 21 | TCHFLGPDCAIEPHD | NITDKI (SEQ ID NO: | QIIHDFV (SEQ ID |
| | WT (SEQ ID NO: 143) | 58) | NO: 59) |
| 22 | LCHILGPDCAIEPHDW | NITDKI (SEQ ID NO: | QIIHDFV (SEQ ID |
| | T (SEQ ID NO: 144) | 58) | NO: 59) |

In some instances, the structurally-stabilized peptide comprises or consists of any one of Constructs 2-6 and 9 of Table 2. In some instances, the structurally-stabilized peptide comprises or consists of any one of Constructs 2-6 and 9 of Table 2 except for at least one (e.g., 1, 2, 3, 4, 5, or 6) amino acid substitution or deletion (e.g., up to a total of 2 to 16 (e.g., 2, 3, 4, 5, 6, 7, 8, 9, 10, 11, 12, 13, 14, 15, 16) substitutions or deletions relative to the sequence of any one of Constructs 2-6 and 9, respectively).

In certain instances, the sequences set forth above in Table 2 can have at least one (e.g., 1, 2, 3, 4, 5, or 6) amino acid substitution or deletion (e.g., up to a total of 2 to 16 (e.g., 2, 3, 4, 5, 6, 7, 8, 9, 10, 11, 12, 13, 14, 15, 16) substitutions or deletions relative to the sequence of SEQ ID NO:10). The EBOV HR2 peptides can include any amino acid sequence described herein.

In some instances, Formula (I) comprising the sequences set forth above in Table 2 can have one or more of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, Formula (I) comprising the sequences set forth above in Table 2 can have one or both of the properties listed below: inhibits infection of a cell by EBOV in pseudovirus and/or live EBOV virus assays and/or prevents infection of a cell by EBOV in the pseudovirus and/or the live EBOV virus assays.

The tether of Formula (I) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is C₁₁ alkyl and x is 6. In some instances of Formula (I), x is 6 and R₃ is Cu alkenyl. In some instances, R₃ is a straight chain alkyl, alkenyl, or alkynyl. In some instances, R₃ is CH₂—CH₂—CH₂—CH═CH—CH₂—CH₂—CH₂.

In one aspect, a structurally-stabilized EBOV HR2 peptide comprises Formula (I), or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1, 2, 3, 4, 5, 6, 7, 8, 9, or 10; and
   (i) each [Xaa]_{w} is TCHILGPDCAIEPHDW (SEQ ID NO:54), [Xaa]ₓ is KNITDK (SEQ ID NO: 55), and each [Xaa]_{y} is DQIIHDFV (SEQ ID NO:56);
   (ii) each [Xaa]_{w} is TCHILGPDCAIEPHDWT (SEQ ID NO:57), [Xaa]ₓ is NITDKI (SEQ ID NO: 58), and each [Xaa]_{y} is QIIHDFV (SEQ ID NO:59);
   (iii) each [Xaa]_{w} is TCHILGPDCAIEPHDWTK (SEQ ID NO:60), [Xaa]ₓ is ITDKID (SEQ ID NO: 61), and each [Xaa]_{y} is IIHDFV (SEQ ID NO:62);
   (iv) each [Xaa]_{w} is TCHILGPDCAIEPHDWTKN (SEQ ID NO:63), [Xaa]ₓ is TDKIDQ (SEQ ID NO: 64), and each [Xaa]_{y} is IHDFV (SEQ ID NO:65);
   (v) each [Xaa]_{w} is TCHILGPDCAIEPHDWTKNI (SEQ ID NO:66), [Xaa]ₓ is DKIDQI (SEQ ID NO: 67), and each [Xaa]_{y} is HDFV (SEQ ID NO:68);
   (vi) each [Xaa]_{w} is TCHILGPDCAIEPHDWTKNIT (SEQ ID NO:69), [Xaa]ₓ is KIDQII (SEQ ID NO: 70), and each [Xaa]_{y} is DFV;
   (vii) each [Xaa]_{w} is TCHILGPDCAIEPHDWTKNITD (SEQ ID NO:72), [Xaa]ₓ is IDQIIH (SEQ ID NO: 73), and each [Xaa]_{y} is FV;
   (viii) each [Xaa]_{w} is TCHILGPDCAIEPHDWTKNITDK (SEQ ID NO:75), [Xaa]ₓ is DQIIHD (SEQ ID NO: 76), and each [Xaa]_{y} is V; or
   (ix) each [Xaa]_{w} is TCHILGPDCAIEPHDWTKNITDKI (SEQ ID NO:77), [Xaa]ₓ is QIIHDF (SEQ ID NO: 78), and each [Xaa]_{y} is absent.

In some instances, the structurally-stabilized peptide (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, Formula (I) comprising the sequences set forth above in Table 2 can have one or both of the properties listed below: inhibits infection of a cell by EBOV in pseudovirus and/or live EBOV virus assays and/or prevents infection of a cell by EBOV in the pseudovirus and/or the live EBOV virus assays. In some instances, wherein R₁ is an alkyl. In some instances, R₁ is a methyl group. In some instances, R₃ is an alkyl. In some instances, R₃ is a methyl group. In some instances, R₂ is an alkenyl. In some instances, z is 1.

In another aspect of Formula (I), the two alpha, alpha disubstituted stereocenters are both in the R configuration or S configuration (e.g., i, i+4 cross-link), or one stereocenter is R and the other is S (e.g., i, i+7 cross-link). Thus, where Formula (I) is depicted as:

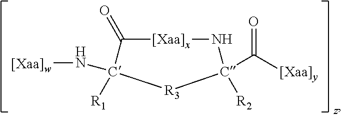

The C′ and C″ disubstituted stereocenters can both be in the R configuration or they can both be in the S configuration. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄-K-R₄]n; and R₄ is a straight chain alkyl, alkenyl, or alkynyl.

As used herein, the term “alkyl,” employed alone or in combination with other terms, refers to a saturated hydrocarbon group that may be straight-chain or branched. In some instances, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some instances, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some instances, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some instances, the alkyl portion has 1 to 4, 1 to 3, 1 to 2, or 1 carbon atom(s). In some instances, the alkyl portion is methylene. In some instances, the cycloalkyl portion has 3 to 10 ring members or 3 to 7 ring members. In some instances, the cycloalkyl group is monocyclic or bicyclic. In some instances, the cycloalkyl portion is monocyclic. In some instances, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some instances, the alkyl portion has 1 to 4, 1 to 3, 1 to 2, or 1 carbon atom(s). In some instances, the alkyl portion is methylene. In some instances, the heteroaryl portion is a monocyclic or bicyclic group having 1, 2, 3, or 4 heteroatoms independently selected from nitrogen, sulfur and oxygen. In some instances, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some instances, halo is F or Cl.

While hydrocarbon tethers are provided herein, other tethers can also be employed in the structurally-stabilized EBOV HR2 peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the EBOV HR2 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A, 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al.,J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+7 are provided herein in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

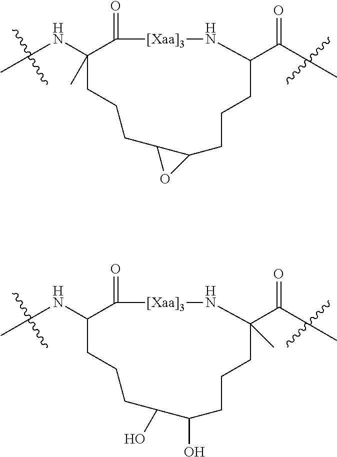

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, alpha disubstituted amino acids are used in the peptide to improve the stability of the alpha helical secondary structure. However, alpha disubstituted amino acids are not required, and instances using mono-alpha substituents (e.g., in the tethered amino acids) are also envisioned.

The structurally-stabilized (e.g., stapled) peptides can include a drug, a toxin, a derivative of polyethylene glycol; a second peptide; a carbohydrate, etc. Where a polymer or other agent is linked to the structurally-stabilized (e.g., stapled) peptide, it can be desirable for the composition to be substantially homogeneous.

The structurally-stabilized (e.g., stapled) peptides can also be modified, e.g., to further facilitate mucoadhesion, membrane binding, or increase in vivo stability, in some instances. For example, acylating or PEGylating a structurally-stabilized peptide increases bioavailability, increases blood circulation, alters pharmacokinetics, alters immunogenicity and/or decreases the needed frequency of administration.

In some instances, the structurally-stabilized (e.g., stapled) peptides disclosed herein have an enhanced ability to bind to or penetrate cell membranes (e.g., relative to non-stabilized peptides). See, e.g., International Publication No. WO 2017/147283, which is incorporated by reference herein in its entirety.

In some instances, the structurally-stabilized peptide is a peptide described in the figures or in the working examples.

In some instances, the structurally-stabilized peptide comprises or consists of the sequence of any one of SEQ ID NOs:80, 85, 86, and 88-92. In some instances, the structurally-stabilized peptide comprises or consists of the sequence of any one of SEQ ID NOs:93, 98, 99, and 101-105.

### Structurally-Stabilized Peptide Conjugates

Also provided herein are conjugates comprising a structurally-stabilized peptide described herein (see Structurally-Stabilized Peptide section above, e.g., a peptide of Table 1 or a construct of Table 2, or a variant thereof) and polyethylene glycol (PEG) and/or cholesterol (or a cholesterol variant, e.g., thiocholesterol). These conjugates have one or more (e.g., 1, 2, 3, 4, 5, 6, 7) of the properties listed below: (i) are alpha-helical; (ii) are protease resistant; (iii) bind to a 5 helix bundle or fusion bundle intermediate of EBOV GP2; (iv) inhibit fusion of EBOV with a host cell; and/or (v) inhibit infection of a cell by EBOV. In some instances, the conjugate inhibits infection of a cell by EBOV in pseudovirus and/or live EBOV virus assays and/or prevents infection of a cell by EBOV in the pseudovirus and/or the live EBOV virus assays. In some instances, the structurally-stabilized peptide of the conjugate comprises or consists of the amino acid sequence of any one of SEQ ID NOs:31-35 and 38. In some instances, the structurally-stabilized peptide of the conjugate comprises or consists of the amino acid sequence of any one of SEQ ID NOs: 40-44 and 47. In some instances, the structurally-stabilized peptide of the conjugate comprises or consists of any one of Constructs 2-6 and 9 of Table 2. In some instances, the structurally-stabilized peptide of the conjugate comprises or consists of the amino acid sequence of any one of SEQ ID NOs:31-35 and 38 except for 1 to 10, 1 to 5, 1 to 3, 2, or 1 amino acid substitutions, insertions, and/or deletions (except at the position of the staple). In some instances, the structurally-stabilized peptide of the conjugate comprises or consists of the amino acid sequence of any one of SEQ ID NOs: 40-44 and 47 except for I to 10, 1 to 5, 1 to 3, 2, or I amino acid substitutions, insertions, and/or deletions (except at the position of the staple). In some instances, the structurally-stabilized peptide of the conjugate comprises or consists of any one of Constructs 2-6 and 9 of Table 2 except for 1 to 10, 1 to 5, 1 to 3, 2, or 1 amino acid substitutions, insertions, and/or deletions (except at the position of the staple). The addition of PEG molecules can improve the pharmacokinetic and pharmacodynamic properties of the structurally-stabilized peptide. For example, PEGylation can reduce renal clearance and can result in a more stable plasma concentration. PEG is a water soluble polymer and can be represented as linked to the peptide as formula:
XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the structurally-stabilized peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG as used herein in some instances functions as a linker or spacer between one of the peptides (e.g., structurally-stabilized peptides of Table 1 or constructs of Table 2) and a cholesterol or thiocholesterol moiety.

In some instances, the PEG molecule includes a cholesterol moiety. In some instances, the cholesterol moiety is thiocholesterol. In some instances, the sulfur of the thioether moiety in thiocholesterol is replaced by an oxygen atom to produce an ether moiety in the cholesterol derivatization.

In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

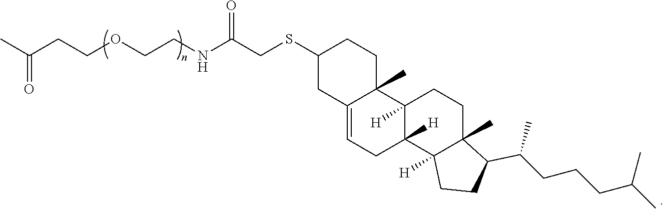

In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

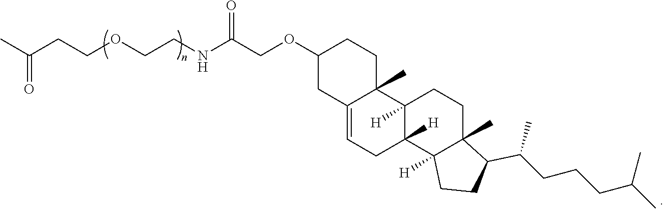

In some instances for each of the formulae above, n=4. In some instances for each of the formulae above, n=5. In some instances for each of the formulae above, n=6. In some instances for each of the formulae above, n=7. In some instances for each of the formulae above, n=8.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain instances, macromolecular polymer (e.g., PEG) is attached to a structurally-stabilized (e.g., stapled) peptide described herein through an intermediate linker. In certain instances, the linker is made up of from 1 to 20 amino acids linked by peptide bonds, wherein the amino acids are selected from the 20 naturally occurring amino acids. Some of these amino acids may be glycosylated, as is well understood by those in the art. In other instances, the 1 to 20 amino acids are selected from glycine, alanine, proline, asparagine, glutamine, and lysine. In other instances, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

Exemplary structurally-stabilized EBOV HR2 peptide conjugates are provided in Table 3, below. In some instances, the structurally-stabilized EBOV HR2 peptide conjugate comprises or consists of the amino acid sequence set forth in any one of SEQ ID NOs:13-17 and 20. In some instances, the structurally-stabilized EBOV HR2 peptide conjugate comprises or consists of the amino acid sequence set forth in any one of SEQ ID NOs:22-26 and 29. In some instances, the structurally-stabilized EBOV HR2 peptide conjugate comprises or consists of the amino acid sequence set forth in any one of SEQ ID NOs:13-17 and 20 except for 1 to 10, 1 to 5, 1 to 3, 2, or 1 amino acid substitutions, insertions, and/or deletions (except at the position of the staple). In some instances, the structurally-stabilized EBOV HR2 peptide conjugate comprises or consists of the amino acid sequence set forth in any one of SEQ ID NOs:22-26 and 29 except for 1 to 10, 1 to 5, 1 to 3, 2, or I amino acid substitutions, insertions, and/or deletions (except at the position of the staple).

TABLE 3
Structurally-Stabilized EBOV HR2 Peptide Conjugates

| | SEQ ID | Sequence | SEQ ID |
|---|---|---|---|
| Sequence | NO | | NO |
| TCHILGPDCAIEPHDW8KNITDKXDQIIHDFV* | 12 | TCHILGPDCAIEPHDW8KNITDKXDQIIHDFV* | 21 |
| TCHILGPDCAIEPHDWT8NITDKIXQIIHDFV* | 13 | TCHILGPDCAIEPHDWT8NITDKIXQIIHDFV* | 22 |
| TCHILGPDCAIEPHDWTK8ITDKIDXIIHDFV* | 14 | TCHILGPDCAIEPHDWTK8ITDKIDXIIHDFV* | 23 |
| TCHILGPDCAIEPHDWTKN8TDKIDQXIHDFV* | 15 | TCHILGPDCAIEPHDWTKN8TDKIDQXIHDFV* | 24 |
| TCHILGPDCAIEPHDWTKNI8DKIDQIXHDFV* | 16 | TCHILGPDCAIEPHDWTKNI8DKIDQIXHDFV* | 25 |
| TCHILGPDCAIEPHDWTKNIT8KIDQIIXDFV* | 17 | TCHILGPDCAIEPHDWTKNIT8KIDQIIXDFV* | 26 |
| TCHILGPDCAIEPHDWTKNITD8IDQIIHXFV* | 18 | TCHILGPDCAIEPHDWTKNITD8IDQIIHXFV* | 27 |
| TCHILGPDCAIEPHDWTKNITDK8DQIIHDXV* | 19 | TCHILGPDCAIEPHDWTKNITDK8DQIIHDXV* | 28 |
| TCHILGPDCAIEPHDWTKNITDKI8QIIHDFX* | 20 | TCHILGPDCAIEPHDWTKNITDKI8QIIHDEX* | 29 |
| TCHILGPDCAIEPHDWT8NITDKIXQIIHDFE* | 106 | TCHILGPDCAIEPHDWT8NITDKIXQIIHDFE* | 119 |
| TCHILGPDCAIEPHDWT8NITDKIXQIIH#FV* | 107 | TCHILGPDCAIEPHDWT8NITDKIXQIIH#FV* | 120 |
| TCHILGPDCAIEPHDWT8NITDKIXQIQHDFV* | 108 | TCHILGPDCAIEPHDWT8NITDKIXQIQHDFV* | 121 |
| TCHILGPDCAIEPHDWTSNITDKIX#IIHDFV* | 109 | TCHILGPDCAIEPHDWT8NITDKIX#IIHDFV* | 122 |
| TCHILGPDCAIEPHDWT8NITDNIXQIIHDFV* | 110 | TCHILGPDCAIEPHDWT8NITDNIXQIIHDEV* | 123 |
| TCHILGPDCAIEPHDWT8NILDKIXQIIHDEV* | 111 | TCHILGPDCAIEPHDWT8NILDKIXQIIHDFV* | 124 |
| TCHILGPDCAIEPHDWT8EITDKIXQIIHDFV* | 112 | TCHILGPDCAIEPHDWT8EITDKIXQIIHDFV* | 125 |
| TCHILGPDCAIEPHDLT8NITDKIXQIIHDFV* | 113 | TCHILGPDCAIEPHDLT8NITDKIXQIIHDFV* | 126 |
| TCHILGPDCAIEPHDET8NITDKIXQIIHDFV* | 114 | TCHILGPDCAIEPHDET8NITDKIXQIIHDFV* | 127 |
| TCHILGPDCASEPHDWT8NITDKIXQIIHDFV* | 115 | TCHILGPDCASEPHDWT8NITDKIXQIIHDFV* | 128 |
| TCHIFGPDCAIEPHDWT8NITDKIXQIIHDFV* | 116 | TCHIFGPDCAIEPHDWT8NITDKIXQIIHDFV* | 129 |
| TCHFLGPDCAIEPHDWT8NITDKIXQIIHDFV* | 117 | TCHFLGPDCAIEPHDWT8NITDKIXQIIHDFV* | 130 |
| LCHILGPDCAIEPHDWT8NITDKIXQIIHDFV* | 118 | LCHILGPDCAIEPHDWT8NITDKIXQIIHDFV* | 131 |

In Table 3, with respect to SEQ ID NOs: 12-20, “8”=α, α-disubstituted non-natural amino acids with olefinic side chain cross-linked to X; “X”=α, α-disubstituted non-natural amino acids with olefinic side chain cross-linked to 8, and * comprises PEG(n)-cholesterol or PEG(n)-thiocholesterol, wherein n=1-36. In Table 3, with respect to SEQ ID NOs: 21-29, “8”=(R)-α-(7′-octenyl)alanine; “X”=(S)-α-(4′-pentenyl)alanine, and *=Lys(epsilon-(PEG)₄-thiocholesterol). In Table 3, # is diaminobutanoic acid. In some instances of SEQ ID NOs:12-20, the * is Lys(epsilon-(PEG)₄-cholesterol) or Lys(epsilon-(PEG)₄-thiocholesterol), wherein n=1-36. In some instances of SEQ ID NOs:12-20, the *=the one of the two formulae shown below, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=4. In some instances, n=8. The two formulae indicated by the “*” include:

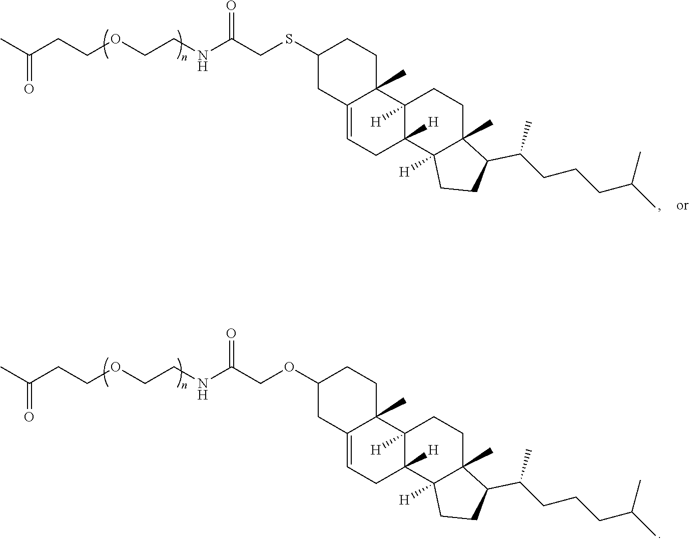

It should be understood that the above peptide conjugates can be modified to include additional amino acids (e.g., 1, 2, 3, 4, 5, 6, 7, 8, 9, 10 amino acids added) at the N and/or C-terminus, and/or to have N and/or C terminal deletions (e.g., 1, 2, 3, 4, 5, 6, 7, 8, 9, 10 amino acids deleted). In some instances, the conjugates are derived from SEQ ID NO:10.

The disclosure encompasses each and structurally-stabilized peptide conjugate listed in Table 3 as well as variants thereof (e.g., having 1, 2, 3, 4, 5, 6, 7, 8, 9, 10, 11, 12, 13, 14, 15, 16) amino acid substitutions, insertions, and/or deletions, except at the positions of the staple, i.e., the bolded residues in Table 3, and at the position of the lipidation (i.e., the * in Table 3)). In some instances, the variant has 1 to 10 amino acid substitutions, insertions, and/or deletions, except at the positions of the staple, (i.e., the bolded residues in Table 3). In some instances, the variant has I to 5 amino acid substitutions, insertions, and/or deletions, except at the positions of the staple, (i.e., the bolded residues in Table 3). In some instances, the variant has 1 to 3 amino acid substitutions, insertions, and/or deletions, except at the positions of the staple (i.e., the bolded residues in Table 3). In some instances, the variant has 1 to 10, 10 to 5, 1 to 3, 2, or 1 amino acid substitutions, insertions, and/or deletions, except at the positions of the staple (i.e., the bolded residues in Table 3), relative to the amino acid sequence set forth in any one of SEQ ID NOs: 22-26 and 29. In some instances, the variant has 1 to 10, 10 to 5, 1 to 3, 2, or 1 amino acid substitutions, insertions, and/or deletions, except at the positions of the staple (i.e., the bolded residues in Table 3), relative to the amino acid sequence set forth in any one of SEQ ID NOs: 13-17 and 20. In some instances, the structurally-stabilized peptide conjugate comprises the N-terminal non-helical portion of EBOV HR2 (e.g., residues 600-612 of SEQ ID NO:1). In some instances, the structurally-stabilized peptide does not comprise amino acids corresponding to positions 634-639 of SEQ ID NO:1. In some instances, the structurally-stabilized peptide conjugate is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide conjugate is 28 to 40 (e.g., 28, 29, 30, 31, 32, 33, 34, 35, 36, 37, 38, 39, 40) amino acids in length. In some instances, the structurally-stabilized peptide conjugate is 28 to 35 (e.g., 28, 29, 30, 31, 32, 33, 34, 35) amino acids in length. In some instances, the structurally-stabilized peptide is 32 amino acids in length. In some instances, the structurally-stabilized peptide conjugate described above have one or more (1, 2, 3, 4, 5, 6) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide conjugate inhibits infection of a cell by EBOV in pseudovirus and/or live EBOV virus assays and/or prevents infection of a cell by EBOV in the pseudovirus and/or the live EBOV virus assays.

In some instances, the structurally-stabilized peptide conjugate includes an amino acid sequence that has 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, or 2 substitutions, insertions, and/or deletions relative to SEQ ID NO: 10. In some instances, a structurally-stabilized peptide conjugate having substitutions, insertions, and/or deletions relative to SEQ ID NO: 10 as described above (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide conjugate inhibits infection of a cell by EBOV in pseudovirus and/or live EBOV virus assays and/or prevents infection of a cell by EBOV in the pseudovirus and/or the live EBOV virus assays.

In some instances, disclosed herein are peptides conjugate that comprise 0-16 (0, 1, 2, 3, 4, 5, 6, 7, 8, 9, 10, 11, 12, 13, 14, 15, 16) amino acid substitutions compared to one of the single-stapled peptides in Table 3. In some instances, disclosed herein are peptides conjugate that are at least 50%, at least 60%, at least 70%, at least 80%, or at least 90% identical to one of the single-stapled peptides in Table 3. In some instances, disclosed herein are peptides that are at least 50%, at least 60%, at least 70%, at least 80%, or at least 90% identical to the amino acid sequence set forth in any one of SEQ ID NOs: 22-26 and 29. In some instances, disclosed herein are peptides that are at least 50%, at least 60%, at least 70%, at least 80%, or at least 90% identical to the amino acid sequence set forth in any one of SEQ ID NOs: 13-17 and 20. It is understood that the variation is not at the staple position (i.e., the bolded residues in Table 3), nor at the site of lipidation (i.e., the * in Table 3). In some instances, disclosed herein are peptides that are 100% identical to one of the single-stapled peptides in Table 3. In some instances, disclosed herein are peptides that are 100% identical to the amino acid sequence set forth in any one of SEQ ID NOs: 22-26 and 29. In some instances, disclosed herein are peptides that are 100% identical to one of the single-stapled peptides in Table 3. In some instances, disclosed herein are peptides that are 100% identical to the amino acid sequence set forth in any one of SEQ ID NOs: 13-17 and 20. In some instances, the structurally-stabilized peptide comprises the N-terminal non-helical portion of EBOV HR2 (e.g., residues 600-612 of SEQ ID NO:1). In some instances, the structurally-stabilized peptide does not comprise amino acids corresponding to positions 634-639 of SEQ ID NO:1. In some instances, the structurally-stabilized peptide is 25 to 60 (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally-stabilized peptide is 28 to 40 (e.g., 28, 29, 30, 31, 32, 33, 34, 35, 36, 37, 38, 39, 40) amino acids in length. In some instances, the structurally-stabilized peptide is 28 to 35 (e.g., 28, 29, 30, 31, 32, 33, 34, 35) amino acids in length. In some instances, the structurally-stabilized peptide is 32 amino acids in length. In some instances, the structurally-stabilized peptide conjugate described above have one or more (1, 2, 3, 4, 5, 6, 7) of the properties listed below: (i) is alpha-helical; (ii) is protease resistant; (iii) binds to a 5 helix bundle or fusion bundle intermediate of EBOV GP2; (iv) inhibits fusion of EBOV with a host cell; and/or (v) inhibits infection of a cell by EBOV. In some instances, the peptide conjugate inhibits infection of a cell by EBOV in pseudovirus and/or live EBOV virus assays and/or prevents infection of a cell by EBOV in the pseudovirus and/or the live EBOV virus assays.

In some instances, any substitution as described herein can be a conservative substitution. In some instances, any substitution as described herein is a non-conservative substitution.

In some instances, a structurally-stabilized peptide conjugate described herein comprises an E (or a conservative substitution of an E) at the amino acid corresponding to position 32 of SEQ ID NO:10. In some instances, a structurally-stabilized peptide conjugate described herein comprises a diaminobutanoic acid (or a conservative substitution of a diaminobutanoic acid) at the amino acid corresponding to position 30 of SEQ ID NO:10. In some instances, a structurally-stabilized peptide conjugate described herein comprises a Q (or a conservative substitution of a Q) at the amino acid corresponding to position 28 of SEQ ID NO:10. In some instances, a structurally-stabilized peptide conjugate described herein comprises a diaminobutanoic acid (or a conservative substitution of a diaminobutanoic acid) at the amino acid corresponding to position 26 of SEQ ID NO:10. In some instances, a structurally-stabilized peptide conjugate described herein comprises an N (or a conservative substitution of an N) at the amino acid corresponding to position 23 of SEQ ID NO:10. In some instances, a structurally-stabilized peptide conjugate described herein comprises an L (or a conservative substitution of an L) at the amino acid corresponding to position 21 of SEQ ID NO:10. In some instances, a structurally-stabilized peptide conjugate described herein comprises an E (or a conservative substitution of an E) at the amino acid corresponding to position 19 of SEQ ID NO:10. In some instances, a structurally-stabilized peptide conjugate described herein comprises an L (or a conservative substitution of an L) at the amino acid corresponding to position 16 of SEQ ID NO:10. In some instances, a structurally-stabilized peptide conjugate described herein comprises an E or an L (or a conservative substitution of an E or an L) at the amino acid corresponding to position 16 of SEQ ID NO:10. In some instances, a structurally-stabilized peptide conjugate described herein comprises an S (or a conservative substitution of an S) at the amino acid corresponding to position 11 of SEQ ID NO:10. In some instances, a structurally-stabilized peptide conjugate described herein comprises an F (or a conservative substitution of an F) at the amino acid corresponding to position 5 of SEQ ID NO:10. In some instances, a structurally-stabilized peptide conjugate described herein comprises an F (or a conservative substitution of an F) at the amino acid corresponding to position 4 of SEQ ID NO:10. In some instances, a structurally-stabilized peptide conjugate described herein comprises an L (or a conservative substitution of an L) at the amino acid corresponding to position 1 of SEQ ID NO:10.

It is understood that the variation is not at a mutated position (i.e., the bolded, italicized residues in Table 3) or is with a conservative amino acid substitution at the mutated position.

In some instances, the conjugate comprises or consists of the sequence of any one of SEQ ID NOs:119, 124, 125, and 127-131. In some instances, the conjugate comprises or consists of the sequence of any one of SEQ ID NOs:106, 111, 112, and 114-118.

### Pharmaceutical Compositions

One or more of any of the structurally-stabilized (e.g., stapled) peptides or structurally-stabilized (e.g., stapled) peptide conjugates described herein can be formulated for use as or in pharmaceutical compositions. The pharmaceutical compositions may be used in the methods of treatment or prevention described herein. In some instances, the pharmaceutical composition comprises a structurally-stabilized peptide described herein and a pharmaceutically acceptable carrier. In some instances, the pharmaceutical composition comprises a structurally-stabilized peptide conjugate described herein and a pharmaceutically acceptable carrier. In certain instances, the pharmaceutical composition comprises a structurally-stabilized (e.g., stapled) peptide or a structurally-stabilized (e.g., stapled) peptide conjugate comprising or consisting of an amino acid sequence that is identical to an amino acid sequence set forth in Table 1, Table 2, or Table 3. In certain instances, the pharmaceutical composition comprises a structurally-stabilized peptide conjugate comprising or consisting of the amino acid sequence of any one of SEQ ID NOs: 22-26 and 29. In certain instances, the pharmaceutical composition comprises a structurally-stabilized peptide conjugate comprising or consisting of the amino acid sequence of any one of SEQ ID NOs: 13-17 and 20. In certain instances, the pharmaceutical composition comprises a structurally-stabilized (e.g., stapled) peptide or a structurally-stabilized (e.g., stapled) peptide conjugate comprising or consisting of an amino acid sequence that is identical to an amino acid sequence set forth in Table 1, Table 2, or Table 3, except for 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide or a structurally-stabilized (e.g., stapled) peptide conjugate comprising or consisting of an amino acid sequence that is identical to the amino acid sequence of any one of SEQ ID NOs: 22-26, 29, 13-17 and 20, except for I to 16, I to 15, I to 14, I to 13, I to 12, I to 11, 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the non-interacting alpha-helical face of these peptides (i.e., to the amino acids that do not interact with the 5 helix bundle or fusion bundle intermediate of EBOV GP2) and/or on the interacting alpha-helical face (i.e., to the amino acids that interact with the 5 helix bundle or fusion bundle intermediate of EBOV GP2). Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays, eye drops, and/or solutions).

In some instances, pharmaceutical compositions can include an effective amount of one or more structurally-stabilized (e.g., stapled) peptides or structurally-stabilized (e.g., stapled) peptide conjugates. The terms “effective amount” and “effective to treat,” as used herein, refer to an amount or a concentration of the described agent (e.g., the structurally-stabilized (e.g., stapled) peptide or structurally-stabilized (e.g., stapled) peptide conjugate) or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this disclosure can include one or more structurally-stabilized (e.g., stapled) peptides or structurally-stabilized (e.g., stapled) peptide conjugates described herein and any pharmaceutically acceptable carrier and/or vehicle. In some instances, pharmaceutical compositions can further include one or more additional therapeutic agents in amounts effective for achieving a modulation of disease or disease symptoms.

The term “pharmaceutically acceptable carrier or adjuvant” refers to a carrier or adjuvant that may be administered to a patient or a subject from another species provided herein, together with a compound of this disclosure (e.g., a structurally-stabilized (e.g., stapled) peptide or structurally-stabilized (e.g., stapled) peptide conjugate), and which does not destroy the pharmacological activity thereof and is nontoxic when administered in doses sufficient to deliver a therapeutic amount of the compound.

In some instances, the pharmaceutical compositions of this disclosure include one or more of acetate, citrate and/or maleate. In some instances, the pharmaceutical compositions can include water or phosphate buffer saline (PBS). In some instance, the pharmaceutical compositions can include chitosan.

The pharmaceutical compositions disclosed herein can include one or more pharmaceutically acceptable salts. In some instances, the pharmaceutically acceptable salts include salts comprising hydrochloride, sodium, sulfate, acetate, phosphate or diphosphate, chloride, potassium, maleate, calcium, citrate, mesylate, nitrate, tartrate, aluminum, gluconate, and any combination thereof.

The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled) peptide or structurally-stabilized (e.g., stapled) peptide conjugate disclosed herein can be further conjugated, for example, to a carrier protein. Such conjugated compositions can be monovalent or multivalent. For example, conjugated compositions can include one structurally-stabilized (e.g., stapled) peptide conjugate disclosed herein conjugated to a carrier protein. Alternatively, as another example, conjugated compositions can include two or more structurally-stabilized (e.g., stapled) peptide conjugates disclosed herein further conjugated to a carrier.

As used herein, when two entities are “conjugated” to one another they are linked by a direct or indirect covalent or non-covalent interaction. In certain instances, the association is covalent. In other instances, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances stability, half-life, tissue exposure, and/or immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

Methods of Making Structurally-Stabilized Peptides and Structurally-Stabilized Peptides Derivatized with PEG(N)-Thiocholesterol or PEG(N)-Cholesterol Moieties

In one aspect, this disclosure features a method of making a structurally-stabilized peptide. In another aspect, this disclosure features a method of making a structurally-stabilized peptide conjugate, e.g., a structurally-stabilized peptide derivatized with PEG(n)-thiocholesterol or PEG(n)-cholesterol moiety(ies). The fully on-resin synthetic method for a structurally-stabilized peptide derivatized with PEG(n)-thiocholesterol or PEG(n)-cholesterol moiety(ies) involves (a) providing a peptide comprising at least two non-natural amino acids with olefinic side chains (e.g., an amino acid sequence described in Table 1 or Table 3), (b) cross-linking the peptide, in some instances by a ruthenium catalyzed metathesis reaction, and (c) derivatizing the C-terminus on resin with a PEG linker of variable length connected to a thiocholesterol or cholesterol moiety. In some instances, step (a) comprises providing a peptide having an amino acid sequence comprising 25-32 contiguous amino acids of the sequence set forth in SEQ ID NO:10 with 2 to 16 amino acid substitutions relative to the sequence set forth in SEQ ID NO:10; wherein two of the 2 to 16 amino acid substitutions are with α, α-disubstituted non-natural amino acids with olefinic side chains at positions of the sequence set forth in SEQ ID NO: 10 selected from (wherein position 1 is the N-terminal threonine and position 32 is the C-terminal valine of SEQ ID NO: 10): (i) positions 17 and 24, (ii) positions 18 and 25, (iii) positions 19 and 26, (iv) positions 20 and 27, (v) positions 21 and 28, (vi) positions 22 and 29, (vii) positions 23 and 30, (viii) positions 24 and 31, or (ix) positions 25 and 32.

Stapled and Stitched Peptide Synthesis: Fmoc-based solid-phase peptide synthesis was used to synthesize stapled peptide fusion inhibitors in accordance with our reported methods for generating all-hydrocarbon stapled peptides (Bird et al., Curr. Protocol. Chem, Biol., 3(3):99-117 (2011; Bird et al., Methods Enzymol., 446:369-86(2008). To achieve the various staple lengths, α-methyl, α-alkenyl amino acids were installed in specific pairings at discrete positions, such as for i, i+7 positioning the use of one S-pentenyl alanine residue (S5) and one R-octenyl alanine residue (R8). For the stapling reaction, Grubbs 1st generation ruthenium catalyst dissolved in dichloroethane was added to the resin-bound peptides. To ensure maximal conversion, three to five rounds of stapling were performed. After appending the PEG(n)-thiocholesterol or PEG(n)-cholesterol moiety (see below), the peptides were then cleaved off of the resin using trifluoroacetic acid, precipitated using a hexane:ether (1:1) mixture, air dried, and purified by LC-MS. All peptides were quantified by amino acid analysis.

Methods of synthesizing the stitched peptides described herein are known in the art. Nevertheless, the following exemplary method may be used. Synthetic chemistry transformations and protecting group methodologies (protection and deprotection) useful in synthesizing the compounds described herein are known in the art and include, for example, those such as described in Bird et al., ACS Chem Biol. (2020) 15(6):1340-1348; Hilinski et al., J Am Chem Soc. (2014) 136(35):12314-22; R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptide sequences of this disclosure can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a linking amino acid may be performed as described in, e.g., Young and Schultz, J Biol Chem. 2010 Apr. 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this disclosure, the amino acid sequence is reverse translated to obtain a nucleic acid sequence encoding the amino acid sequence, preferably with codons that are optimal for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Gyros Protein Technologies. Peptide bonds can be replaced, e.g., to increase physiological stability of the peptide, by: a retro-inverso bonds (C(O)—NH); a reduced amide bond (NH—CH2); a thiomethylene bond (S—CH2 or CH2-S); an oxomethylene bond (O—CH2 or CH2-O); an ethylene bond (CH2-CH2); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH3; and a fluoro-ketomethylene bond (C(O)—CFR or CFR—C(O) wherein R is H or F or CH3.

The peptides can be further modified by: acetylation, amidation, biotinylation, cinnamoylation, farnesylation, fluoresceination, formylation, myristoylation, palmitoylation, and other lipidation, specifically including thiocholesterol or cholesterol modification using the on-resin method disclosed herein, phosphorylation (Ser, Tyr or Thr), stearoylation, succinylation and sulfurylation. As indicated above, peptides can be conjugated to or contain linker atoms or moeities of variable length, for example, polyethylene glycol (PEG) moieties of variable length; alkyl groups (e.g., C₁-C₂₀ straight or branched alkyl groups); fatty acid radicals; and combinations thereof. α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). In some instances the stitched peptide comprises a kinkage between i, i+4, and i+4 and i+8. In some instances, the amino acids forming the staple or stitch are (R)-2-(4′-pentenyl)Alanine, 2,2-bis(4-pentenyl)glycine, and (S)-2-(4′-pentenyl)Alanine at positions i, i+4, and i+8, respectively, of the stitch. In some instances for peptides where an i linked to i+7, i+7 linked to i+14 stitch is used (four turns of the helix stabilized): one R-octenyl alanine (e.g., (R)-α-(7′-octenyl)alanine), one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin or Rink Amide AM resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched or non-stapled peptide contaminants or are isolated. Methods for purifying peptides include, for example, synthesizing the peptide on a solid-phase support. Following cyclization, multiple alternative solvent and purification schemes are known in the art for peptide and stapled peptide isolation and purification and may use solvents that include, but are not limited to, DMSO, DMSO/dichloromethane mixture, DMSO/NMP mixture, or a mixture/solution that does not include DMSO. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

C-terminal derivatization of stapled or stitched peptides with PEG(n)-thiocholesterol or PEG(n)-cholesterol using an on-resin synthetic approach: To generate the carboxy thiocholesterol or carboxy cholesterol reagent for peptide derivatization by solid phase synthesis, thiocholesterol or cholesterol was dissolved in dichloromethane (DCM) at 0.1 M and added to a round bottom flask. 3 eq of a base (diisopropylethylamine for thiocholesterol or sodium hydride for cholesterol) was added with stirring. 5 eq of the t-butyl ester of bromoacetic acid was added next and the reaction was stirred for 2 hours at room temperature followed by 30 min at 40° C. Two volumes of trifluoroacetic acid (relative to DCM) was added and the reaction was stirred at room temperature for 30 min. The reaction progress was monitored by TLC (19:1 Hex:EtOAc) with KMnO₄ staining. Thiocholesterol, for example, migrated with the solvent front with thioether slowing migration by ˜20% and TFA hydrolysis brought the spot to baseline. The reaction mixture was added to 5 vol water and the 1 vol DCM was added. The DCM layer was washed with 0.1M HCl, brine and dried with sodium sulfate. Removal of the solvent by Rotovap yielded an orange heavy oil that was used without further purification. The yield was near quantitative. Purity was determined to be greater than 90% by NMR of the olefin proton vs the new CH2 singlet. For peptide derivatization with thiocholesterol or cholesterol, the completed resin bound peptide sequence was treated with 20% piperidine/DMF followed by capping with acetic anhydride to block the N-terminal amine before the C-terminal side chain lysine amine was revealed by treatment with 2% hydrazine in DMF, 5× for 10 min each. The amine was acylated with an Fmoc-protected PEG(n) amino acid (e.g., n=1-36 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36)) at which point the olefins were crosslinked by treating with Grubbs(I) catalyst, 3× for 2 h each. Upon completion, the Fmoc was removed from the C-terminal NH of the PEG reagent and the amine was acylated with carboxy-thiocholesterol (or carboxy-cholesterol) for 30 min. TFA cleavage yielded a crude product of excellent purity that was further purified using semi-prep HPLC.

Properties of the stitched or stapled peptides derivatized with a C-terminal PEG(n)-thiocholesterol or PEG(n)-cholesterol of the disclosure can be assayed, for example, using the methods described below and in the Examples.

Methods of Treatment and/or Prevention

The disclosure features methods of using any of the structurally-stabilized (e.g., stapled) peptides or structurally-stabilized peptide conjugates (or pharmaceutical compositions comprising said structurally-stabilized peptides or structurally-stabilized peptide conjugates) described herein for the prevention and/or treatment of an EBOV infection or EBOV disease. The terms “treat” or “treating,” as used herein, refers to alleviating, inhibiting, or ameliorating the disease or infection from which the subject (e.g., human) or other species (e.g., pets; farm animals; domestic animals) is suffering. In some instances, the subject is an animal. In some instances, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat or other species that spread EBOV (e.g., a nonhuman primate or a fruit bat). In some instances, the subject is a human. In certain instances, such terms refer to a non-human animal (e.g., a non-human animal such as a pig, horse, cow, cat or dog). In some instances, such terms refer to a pet or farm animal. In some instances, such terms refer to a human.

The structurally-stabilized (e.g., stapled) peptides or structurally-stabilized peptide-conjugates (or pharmaceutical compositions comprising the same) described herein can be useful for treating a subject (e.g., human subject or a species as described above) having an EBOV infection. The structurally-stabilized (e.g., stapled) peptides or structurally-stabilized peptide conjugates (or pharmaceutical compositions comprising the same) described herein can also be useful for treating a subject (e.g., human subject or a species as described above) having an EBOV disease. The structurally-stabilized (e.g., stapled) peptides or structurally-stabilized peptide conjugates (or pharmaceutical compositions comprising the same) described herein can also be useful for treating a subject having an EBOV disease, wherein the subject is a mammal such as a non-human primate or a fruit bat. In some instances, the structurally-stabilized peptide (or a pharmaceutical composition comprising the same) is used in treatment of an EBOV infection or disease. In some instances, the structurally-stabilized peptide conjugate (or a pharmaceutical composition comprising the same) is used in treatment of an EBOV infection or disease.

The structurally-stabilized (e.g., stapled) peptides or structurally-stabilized peptide-conjugates (or pharmaceutical compositions comprising the same) described herein can be useful for preventing a subject (e.g., human subject or a species as described above) from having an EBOV infection. The structurally-stabilized (e.g., stapled) peptides or structurally-stabilized peptide-conjugates (or pharmaceutical compositions comprising the same) described herein can be useful for preventing a subject (e.g., human subject or a species as described above) from having an EBOV disease. In some instances, the subject is a human. In some instances, the subject is a non-human primate. In some instances, the subject is a fruit bat.

Thus, provided herein is a method of treating an ebolavirus infection in a subject (e.g., a human, non-human primate, or a fruit bat) in need thereof, the method comprising administering to the subject a therapeutically effective amount of a structurally-stabilized peptide described herein (or a pharmaceutical composition comprising the structurally-stabilized peptide).

Also provided herein is a method of treating an ebolavirus infection in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

Also provided herein is a method of preventing an ebolavirus infection in a subject (e.g., a human, non-human primate, or a fruit bat) in need thereof, the method comprising administering to the subject a therapeutically effective amount of a structurally-stabilized peptide described herein (or a pharmaceutical composition comprising the structurally-stabilized peptide).

Also provided herein is a method of preventing an ebolavirus infection in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

Also provided herein is a method of treating an ebolavirus disease in a subject (e.g., a human, non-human primate, or a fruit bat) in need thereof, the method comprising administering to the subject a therapeutically effective amount of a structurally-stabilized peptide described herein (or a pharmaceutical composition comprising the structurally-stabilized peptide).

Also provided herein is a method of treating an ebolavirus disease in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

Also provided herein is a method of preventing an ebolavirus disease in a subject (e.g., a human, non-human primate, or a fruit bat) in need thereof, the method comprising administering to the subject a therapeutically effective amount of a structurally-stabilized peptide described herein (or a pharmaceutical composition comprising the structurally-stabilized peptide).

Also provided herein is a method of preventing an ebolavirus disease in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

In certain instances, the EBOV infection is a Zaire ebolavirus infection. In certain instances, the EBOV disease is caused by a Zaire ebolavirus infection. In certain instances, the EBOV infection is a Bundibugyo ebolavirus infection. In certain instances, the EBOV disease is caused by a Bundibugyo ebolavirus infection. In certain instances, the EBOV infection is a Sudan ebolavirus infection. In certain instances, the EBOV disease is caused by a Sudan ebolavirus infection. In certain instances, the EBOV infection is a Tai Forest ebolavirus infection. In certain instances, the EBOV disease is caused by a Tai Forest ebolavirus infection.

The disclosure also features methods of using any of the structurally-stabilized (e.g., stapled) peptides or structurally-stabilized peptide conjugates (or pharmaceutical compositions comprising said structurally-stabilized peptides or structurally-stabilized peptide conjugates) described herein for the prevention and/or treatment of a Marburg virus infection, a Bombali ebolavirus infection, or a Mengla dianlovirus infection. In some instances, the subject is an animal. In some instances, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat or other species that spread Marburg virus, Bombali ebolavirus, or Mengla dianlovirus (e.g., a nonhuman primate or a fruit bat). In some instances, the subject is a human. In certain instances, such terms refer to a non-human animal (e.g., a non-human animal such as a pig, horse, cow, cat or dog). In some instances, such terms refer to a pet or farm animal. In some instances, such terms refer to a human.

Also provided herein is a method of treating a Marburg virus infection, a Bombali ebolavirus infection, or a Mengla dianlovirus infection in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

Also provided herein is a method of treating a Marburg virus infection, a Bombali ebolavirus infection, or a Mengla dianlovirus infection in a subject (e.g., a human, non-human primate, or a fruit bat) in need thereof, the method comprising administering to the subject a therapeutically effective amount of a structurally-stabilized peptide described herein (or a pharmaceutical composition comprising the peptide).

Also provided herein is a method of treating a Marburg virus infection, a Bombali ebolavirus infection, or a Mengla dianlovirus infection in a subject (e.g., a human, non-human primate, or a fruit bat) in need thereof, the method comprising administering to the subject a therapeutically effective amount of a structurally-stabilized peptide conjugate described herein (or a pharmaceutical composition comprising the conjugate).

Also provided herein is a method of treating a Marburg virus infection, a Bombali ebolavirus infection, or a Mengla dianlovirus infection in a subject (e.g., a human, non-human primate, or a fruit bat) in need thereof, the method comprising administering to the subject a therapeutically effective amount of a structurally-stabilized peptide described herein (or a pharmaceutical composition comprising the peptide).

In certain instances, the subject (e.g., human, non-human primate, or fruit bat) is administered a peptide described in Table 1, or a variant thereof, a construct described in Table 2, or a variant thereof, or a conjugate described in Table 3, or a variant thereof. In certain instances, the subject (e.g., human, non-human primate, or fruit bat) is administered a structurally-stabilized peptide conjugate comprising or consisting of the amino acid sequence set forth in any one of SEQ ID NOs:13-17, 20, 22-26, and 29. In certain instances, the subject (e.g., human, non-human primate, or fruit bat) is administered a structurally-stabilized peptide conjugate comprising or consisting of the amino acid sequence set forth in any one of SEQ ID NOs:13-17, 20, 22-26, and 29, or a variant thereof (e.g., having 1 to 10, 1 to 5, 1 to 3, 2, or 1 amino acid substitutions, insertions, and/or deletions relative to the amino acid sequence set forth in any one of SEQ ID NOs:13-17, 20, 22-26, and 29, respectively, wherein the amino acid substitution(s) and/or deletion(s) is/are not at the staple positions). In certain instances, the subject (e.g., human, non-human primate, or fruit bat) is administered a structurally-stabilized peptide described in the section Structurally-Stabilized Peptides above. In some instances, the subject (e.g., human, non-human primate, or fruit bat) is administered a structurally-stabilized peptide conjugate described in the section Structurally-Stabilized Peptide Conjugates above. In some instances, the subject is administered a structurally-stabilized peptide or a structurally-stabilized peptide conjugate described in the figures or working examples.

In some instances of a method involving an EBOV disease or infection, the subject (e.g., human, non-human primate, or fruit bat) is infected with an EBOV. In some instances of a method involving an EBOV disease or infection, the subject (e.g., human, non-human primate, or fruit bat) is at risk of being infected with an EBOV. In some instances of a method involving an EBOV disease or infection, the subject (e.g., human, non-human primate, or fruit bat) is at risk of developing an EBOV disease. In some instances, a subject (e.g., human, non-human primate, or fruit bat) is at risk of being infected with an EBOV or at risk of developing an EBOV disease if the subject lives in an area (e.g., city, state, country) subject to an active EBOV outbreak (e.g., an area where at least 1, at least 2, at least 3, at least 4, at least 5, at least 6, at least 7, at least 8, at least 9, at least 10, at least 20, at least 30, at least 40, or more subjects have been diagnosed as infected with an EBOV or having an EBOV disease). In some instances, a subject (e.g., human, non-human primate, or fruit bat) is at risk of being infected with an EBOV or developing an EBOV disease if the subject lives in an area near (e.g., a bordering city, state, country) a second area (e.g., city, state, country) subject to an active EBOV outbreak (e.g., an area near (e.g., bordering) a second area where at least 1, at least 2, at least 3, at least 4, at least 5, at least 6, at least 7, at least 8, at least 9, at least 10, at least 20, at least 30, at least 40, or more subjects have been diagnosed as infected with a EBOV or having an EBOV disease). In certain instances, the EBOV disease is caused by a Zaire ebolavirus infection. In certain instances, the EBOV disease is caused by a Bundibugyo ebolavirus infection. In certain instances, the EBOV disease is caused by a Sudan ebolavirus infection. In certain instances, the EBOV disease is caused by a Tai Forest ebolavirus infection.

In general, methods include selecting a subject and administering to the subject an effective amount of one or more of the structurally-stabilized (e.g., stapled) peptides or structurally-stabilized (e.g., stapled) peptide conjugates described herein, e.g., in or as a pharmaceutical composition, and optionally repeating administration as required for the prevention or treatment of the infection or disease (e.g., the EBOV infection or the EBOV disease) and can be administered orally, intranasally, intravenously, subcutaneously, intramuscularly, or topically, including skin, nasal, sinus, ocular, oropharynx, respiratory tree, and lung administration. In some instances, the administration is by a topical respiratory application which includes application to the nasal mucosa, sinus mucosa, oropharyngeal mucosa, or respiratory tree, including the lungs. In some instances, topical application includes application to the skin or eyes. A subject can be selected for treatment based on, e.g., determining that the subject is at risk to acquire or has an EBOV infection. The peptides and conjugates of this disclosure can be used to determine if a subject is infected with an EBOV. In some instances, the conjugates described herein increase bioavailability, increase blood circulation, alter pharmacokinetics, decrease immunogenicity and/or decrease the needed frequency of administration.

Specific dosage and treatment regimens for any particular patient or subject will depend upon a variety of factors, including the activity of the specific compound employed, the age, body weight, general health status, sex, diet, time of administration, rate of excretion, drug combination, the severity and course of the disease, condition or symptoms, the patient's or subject's disposition to the disease, condition or symptoms, and the judgment of the treating physician or veterinarian.

An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week, including once every other day. The skilled artisan will appreciate that certain factors may influence the dosage and timing required to effectively treat a subject, including but not limited to the risk to acquire or severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

In some instances, the conjugate comprises or consists of the sequence of any one of SEQ ID NOs:119, 124, 125, and 127-131. In some instances, the conjugate comprises or consists of the sequence of any one of SEQ ID NOs:106, 111, 112, and 114-118. In some instances, the structurally-stabilized peptide comprises or consists of the sequence of any one of SEQ ID NOs:80, 85, 86, and 88-92. In some instances, the structurally-stabilized peptide comprises or consists of the sequence of any one of SEQ ID NOs:93, 98, 99, and 101-105.

### EXAMPLES

### Example 1: Design and Synthesis of Stapled EBOV HR2 Peptides Derivatized with C-Terminal PEG(N)-Thiocholesterol or PEG(N)-Cholesterol Moieties

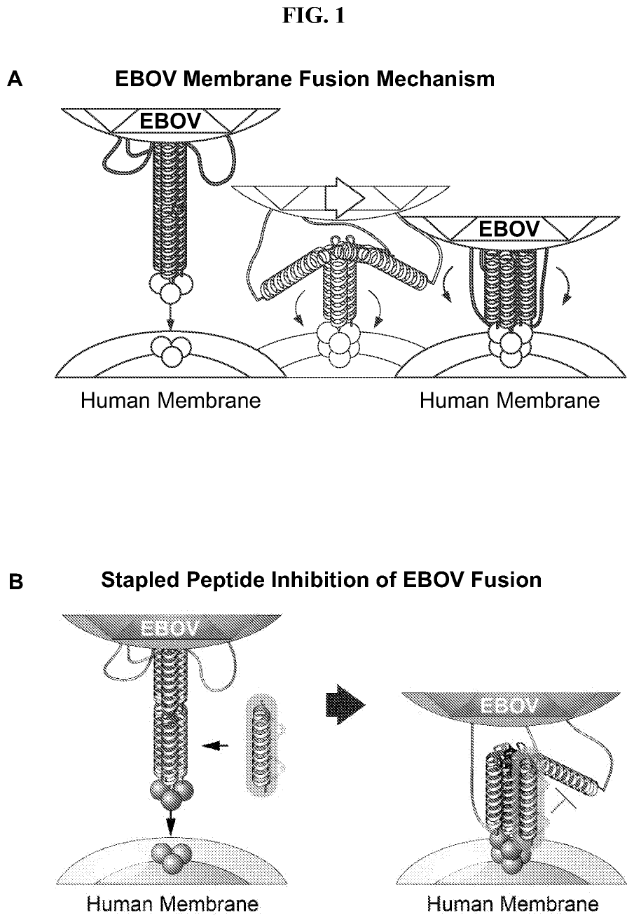

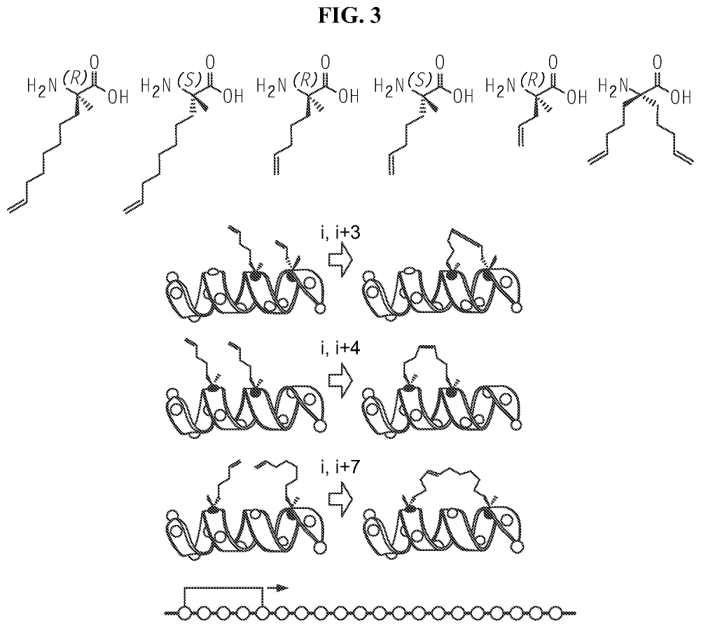

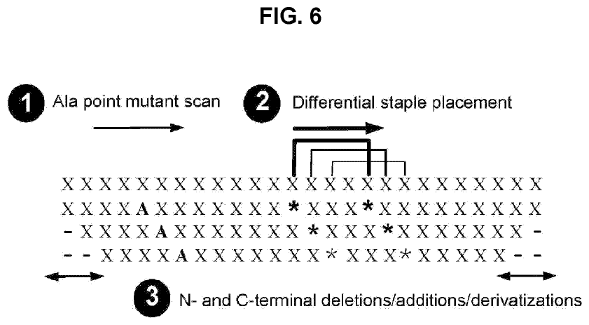

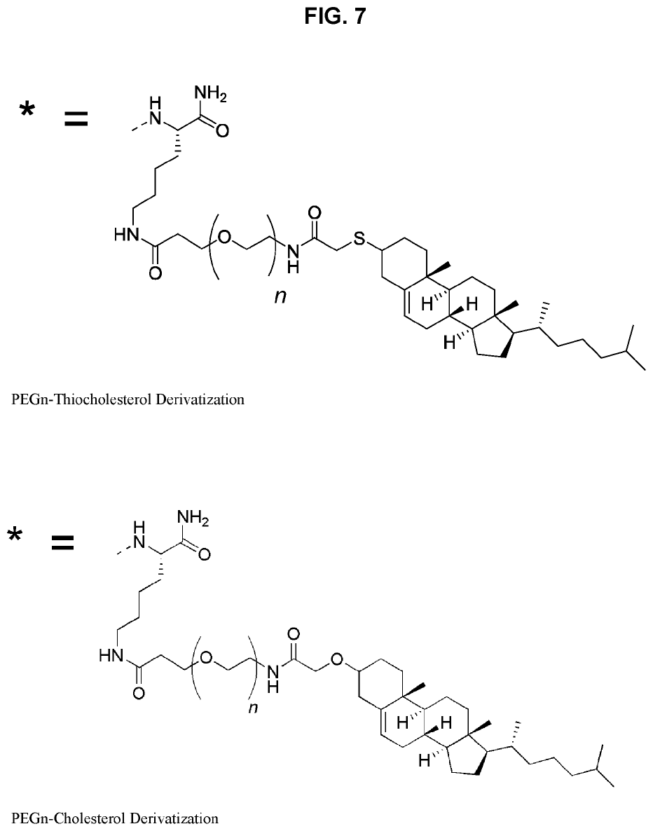

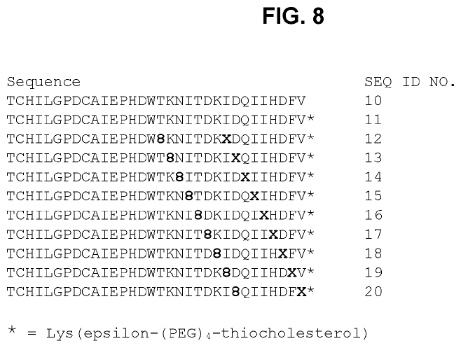

To design stapled lipopeptides peptides that could block the fusion of Ebolaviruses to a host cell (FIG. 1), a series of stapled peptides bearing differentially localized chemical staples and derivatized with PEG(n)-thiocholesterol or PEG(n)-cholesterol moieties at the C-termini were designed and then synthesized on resin by solid phase synthesis. The differentially localized chemical staples were located within the EBOV HR2 domain (i.e., amino acids 600-631 of SEQ ID NO:1 with a C₆₀₉A mutation) of the sequence of the surface (S) glycoprotein (GP2) of the Ebolavirus (see, FIG. 2A and FIG. 2B), and preferably within the alpha-helical region (i.e., amino acids 613-631 of SEQ ID NO:1 (FIG. 2C and FIG. 2D) and its variants; see, FIG. 2B), by replacing native residues with α, α-disubstituted non-natural olefinic residues (e.g., “X” for S-pentenyl alanine and “8” for R-octenyl alanine installed at select i, i+7 positions). It is possible to form combinations thereof in the form of double staples or stitches, followed by ruthenium-catalyzed olefin metathesis (see, FIGS. 3-5). In this study, (R)-α-(7′-octenyl)alanine was installed at position i and (S)-α-(4′-pentenyl)alanine was installed at position i+7 (see, FIG. 8). This approach to designing, synthesizing, and identifying optimal stapled peptide constructs to target the EBOV fusion apparatus includes the generation of Ala scan (e.g., mutants), staple scan, and variable N- and C-terminal deletion, addition, and derivatization libraries (see, FIG. 6) for conjugation to PEG-thiocholesterol or PEG-cholesterol moieties (see, FIG. 7).

Stapled EBOV HR2 constructs bearing C-terminal derivatization with PEG(n)-thiocholesterol or PEG(n)-cholesterol moieties were designed by replacing two naturally occurring amino acids with the non-natural (R)-2-(((9H-fluoren-9-yl)methoxy)carbonylamino)-2-methyl-dec-9-enoic acid (Fmoc-R8) and S-2-(4′-pentenyl) alanine (S5) amino acids at i, i+7 positions (i.e. flanking 7 amino acids) to generate a staple spanning two α-helical turns (FIG. 8). Asymmetric syntheses of α, α-disubstituted amino acids were performed as previously described in detail (Schafmeister et al., J. Am. Chem. Soc., 2000; Walensky et al., Science, 2004; Bird et al., Current Protocols in Chemical Biology, 2011, each of which is incorporated by reference in its entirety).

“Staple scanning” was performed to respectively identify residues and binding surfaces critical for interaction, which dictates the design of optimized constructs and negative control mutants (see, FIG. 3). The peptide N-termini were capped with acetyl or a fluorophore (e.g., FITC, rhodamine), depending upon the experimental application.

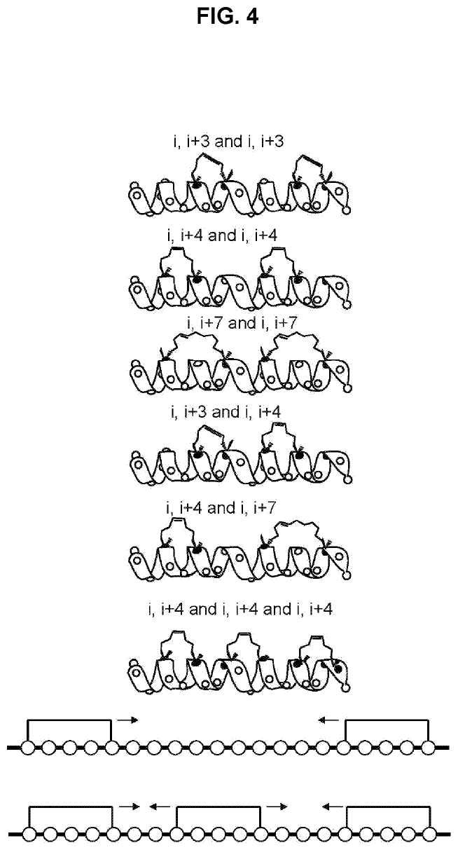

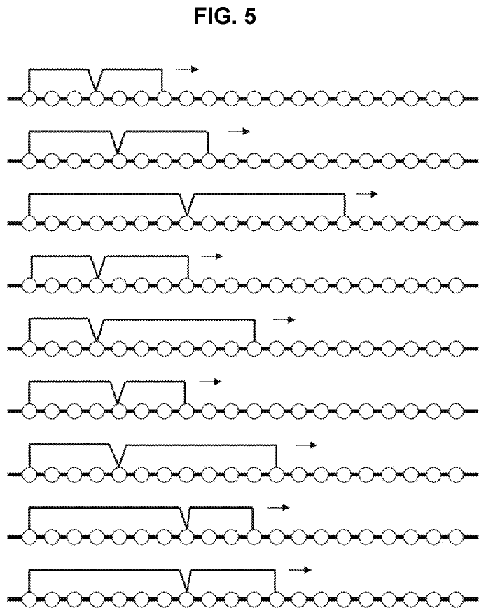

Doubly stapled peptides are generated by installing two-S5-S5, two R8-S5, or other combinations of crosslinking non-natural amino acids (see, FIG. 4). Multiply stapled or stitched peptides are generated using similar principles (see, FIGS. 4-5).

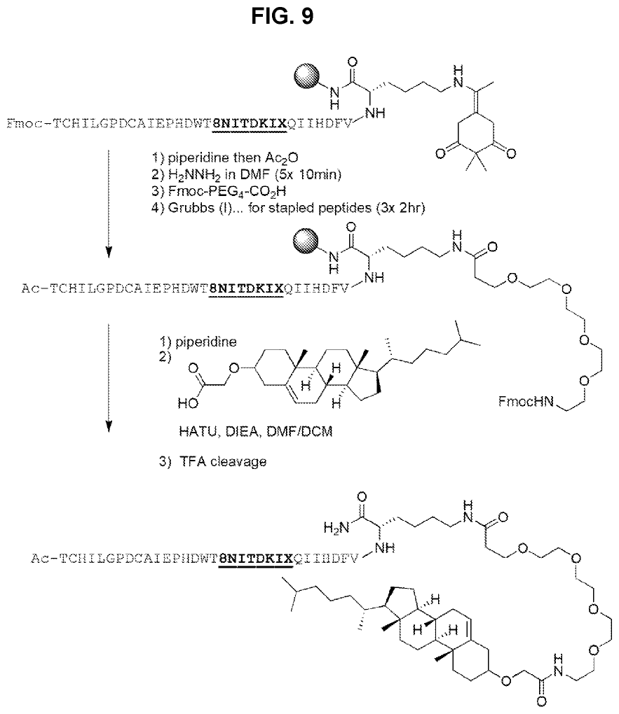

To enable peptide derivatization with thiocholesterol or cholesterol on resin, carboxy-thiocholesterol or carboxy-cholesterol were synthesized according to the procedure described above (see Methods of Making Structurally-Stabilized Peptides and Structurally-Stabilized Peptides Derivatized with PEG(n)-Thiocholesterol or PEG(n)-Cholesterol Moieties section above). The completed resin-bound peptide was capped with an acetyl group (by use of acetic anhydride) followed by deprotection of the C-terminal side chain lysine amine by treatment with 2% hydrazine. The amine was acylated with an Fmoc-protected PEG(n) amino acid (e.g., n=1-36) at which point the olefins were crosslinked by treating with Grubbs(I) catalyst. The Fmoc was removed from the C-terminal NH of the PEG(n) amino acid and the amine acylated with carboxy-thiocholesterol or carboxy-cholesterol. The final peptide product was obtained after peptide deprotection and cleavage, and purification by reverse phase high performance liquid chromatography/mass spectrometry (LC/MS). See the full synthetic schema in FIG. 9. Exemplary i, i+7 stapled EBOV HR2 peptides derivatized with PEG(n)-thiocholesterol generated by use of this synthetic schema are listed in FIG. 8.

### Example 2: Identifying Optimally Stapled EBOV HR2 Peptides Bearing a C-Terminal Peg4-Thiocholesterol that Block Live Ebolavirus Infection

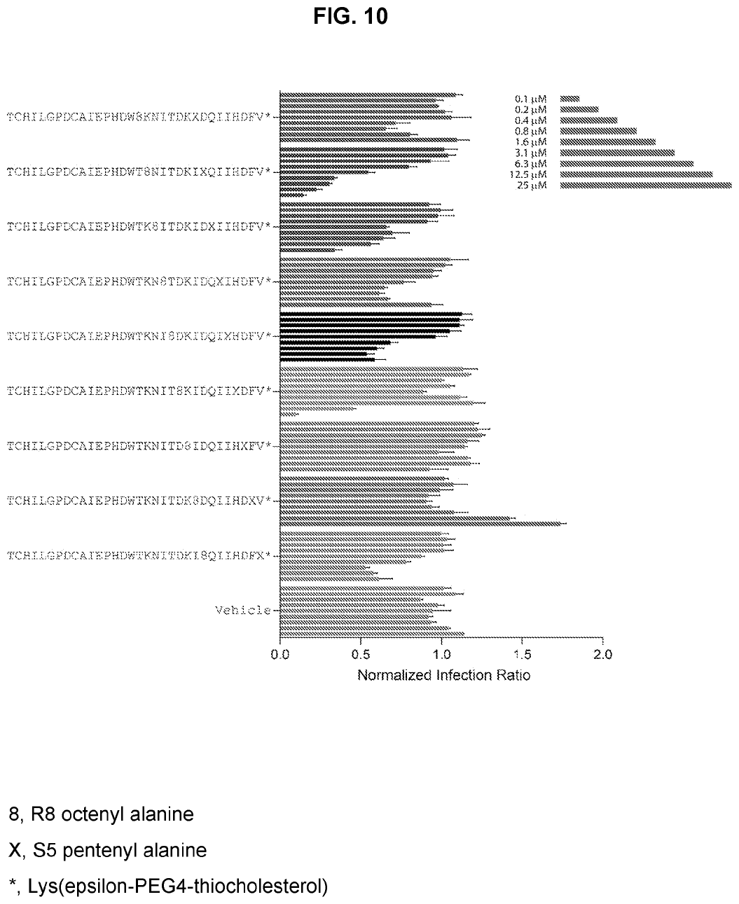

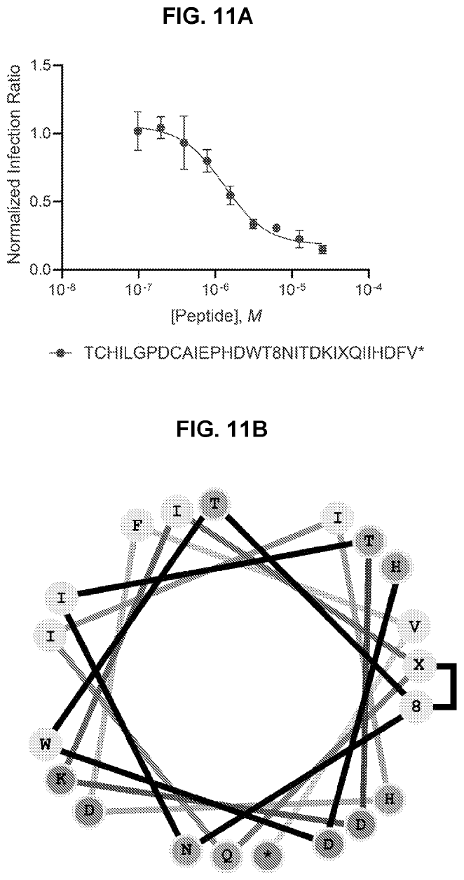

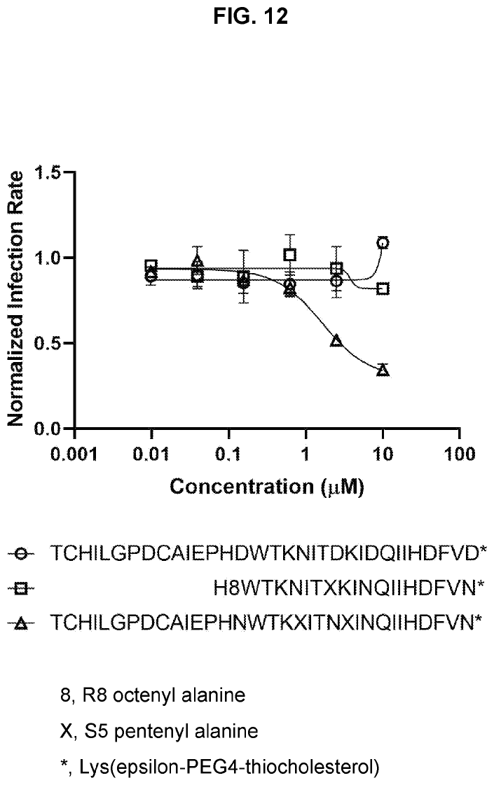

To test the antiviral activity of the structurally-stabilized peptide conjugates against EBOV, HeLa cells were plated (4×10³) in a 384-well plate and grown overnight. The next day, cells were treated with structurally-stabilized EBOV HR2 peptides or structurally-stabilized EBOV HR2 peptide conjugates (starting at 25 μM with 2-fold serial dilutions) in triplicate to yield a 9-point dose curve. Each well was infected in a BSL-4 laboratory at the National Emerging Infectious Diseases Laboratories (NEIDL) with wild type EBOV at a multiplicity of infection (MOI) of 0.3. Cells were incubated with virus for 24 hours, at which point they were fixed by immersion into formalin overnight at 4° C. The formalin was removed and plates were washed three times with phosphate buffered saline (PBS). Cells were stained with EBOV GP specific antibody 4F3 (IBT Bioservices, MD, USA) followed by Alexa546 secondary antibody. Cell nuclei were stained using Hoechst at 1:50 000, and plates were imaged using a Cytation 1 (Biotek, VT, USA) automated microscope and nuclei and infected cells were counted using Cell Profiler software (Broad Inst. MA, USA). Infection efficiency was calculated as the ratio of infected to cell nuclei and normalized to vehicle (0.2% DMSO) treated controls. An i, i+7 stapled lipopeptide library (SEQ ID NOs:21-29) of an exemplary 32-amino acid long HR2 sequence of EBOV (SEQ ID NO: 10) was tested in the live EBOV assay and revealed differential binding activity, with a subset of peptides exhibiting dose-responsive anti-viral activity (specifically, SEQ ID NOs: 22, 23, 24, 25, 26, and 29) (FIG. 10). A lead construct (SEQ ID NO: 22) bearing a staple at the non-interacting surface of the HR2 alpha-helix demonstrated an IC50 of ˜1.5 micromolar (FIG. 11A and FIG. 11B). An unstapled lipopeptide of SEQ ID NO: 7 (based on an HR2 sequence corresponding to SEQ ID NO: 156) exhibited no anti-EBOV activity in the live virus assay, yet insertion of a single staple in the helical region of SEQ ID NO:7 conferred antiviral activity (FIG. 12). Notably, a stapled lipopeptide of shortened sequence (relative to SEQ ID NO:7) that excluded the non-helical region of the EBOV HR2 sequence was inactive in this live virus assay (FIG. 12). These data demonstrate the surprising finding that antiviral activity of a structurally-stabilized EBOV HR2 peptide conjugate depends on at least part of the non-helical portion of HR2 (e.g., residues 600-612 of SEQ ID NO:1) and structural stabilization in the helical, C-terminal portion of HR2 (e.g., residues 613-632 of SEQ ID NO:1). See FIG. 2C, FIG. 10, FIG. 11A, FIG. 11B, and FIG. 12.

### Example 3: Mutated Stapled EBOV HR2 Peptides Bearing a C-Terminal Peg4-Thiocholesterol

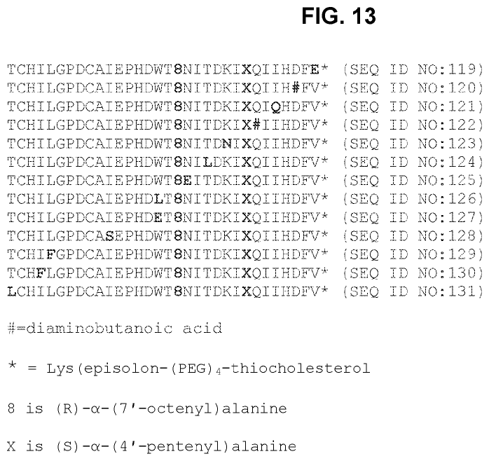

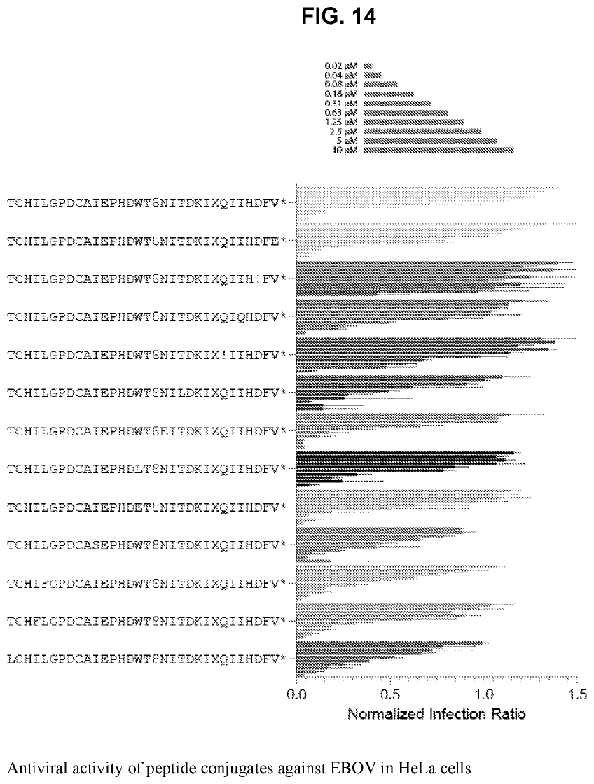

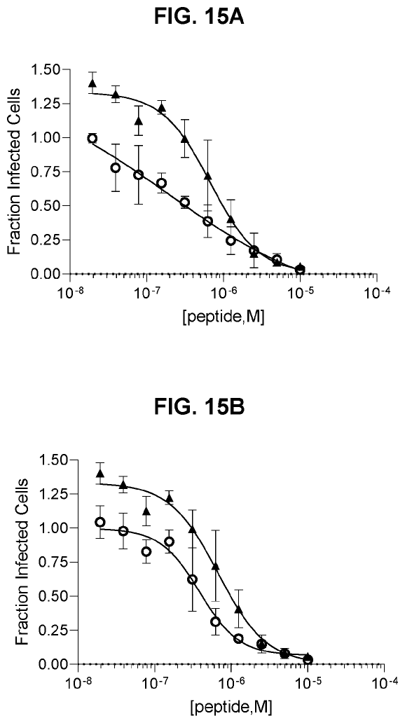

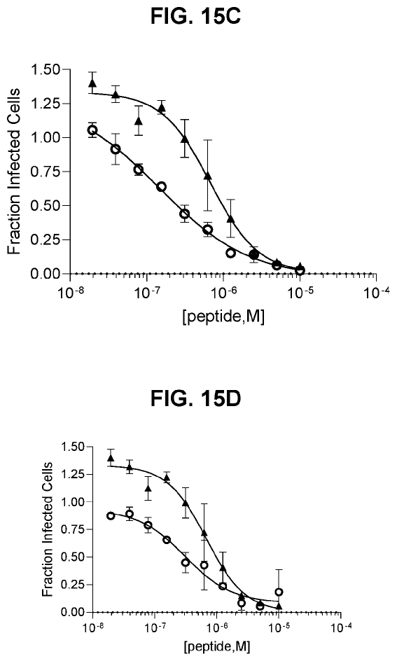

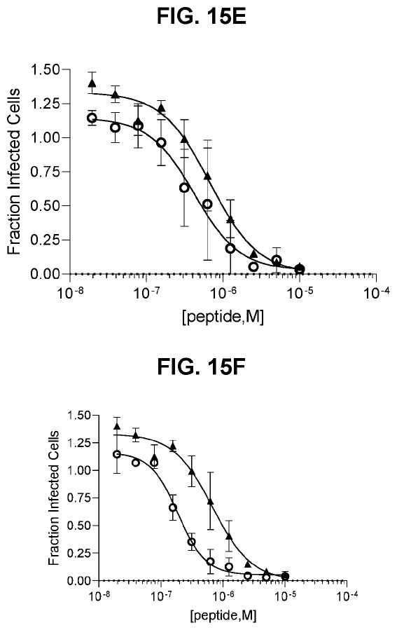

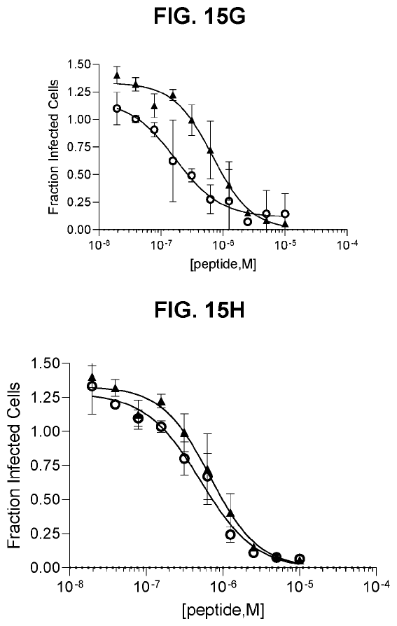

Point mutations were introduced into the structurally-stabilized peptide conjugate of SEQ ID NO:22 (FIG. 13) and the antiviral activity of the resulting structurally-stabilized peptide conjugates was determined as described for FIG. 10 in Example 2. A dose-responsive anti-viral activity was observed (FIG. 14 and FIGS. 15A-15H).

### Example 4: Stapled EBOV HR2 Peptides Bearing a C-Terminal Peg4-Thiocholesterol for Use Against Pseudotyped Marburg Virus

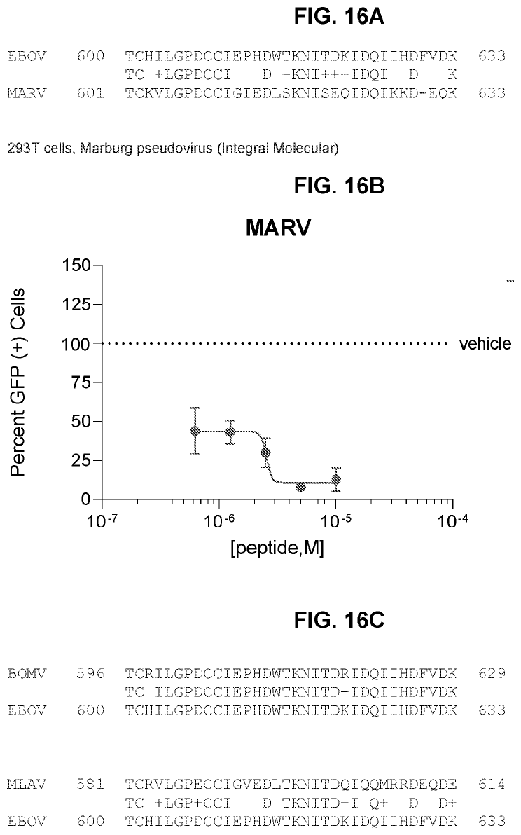

A sequence alignment of an HIR2 domain of a Marburg Virus and an Ebola Virus (Zaire strain) was performed (FIG. 16A).

The antiviral activity of an exemplary i, i+7 stapled EBOV HR2 peptide having the sequence of SEQ ID NO:22 was examined in a pseudovirus assay (pseudovirus: Integral Molecular RVP-1501, Marburg Uganda 2007; cells: 293T-ACE2; peptides: serial 2-fold dilution starting at 10 μM; read-out: 72 hours). An antiviral effect was observed against the Marburg virus pseudoparticles (FIG. 16B).

Sequence alignments of an HR2 domain of Bombali ebolavirus and an Ebola Virus (Zaire strain) and of an HR2 domain of Mengla dianlovirus and an Ebola Virus (Zaire strain) were also performed (FIG. 16C).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A conjugate comprising a structurally-stabilized peptide and polyethylene glycol (PEG) and/or cholesterol or thiocholesterol;
wherein the PEG and/or cholesterol or thiocholesterol are linked to the C-terminal amino acid of the structurally-stabilized peptide;
wherein the structurally-stabilized peptide comprises:
(a) an amino acid sequence comprising 28-32 contiguous amino acids of the sequence set forth in SEQ ID NO:10 with two to five amino acid substitutions relative to the sequence set forth in SEQ ID NO:10;
wherein two of the two to five amino acid substitutions are with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32; or
(b) an amino acid sequence comprising 28-32 contiguous amino acids of the sequence set forth in SEQ ID NO:10 with two to five amino acid substitutions relative to the sequence set forth in SEQ ID NO:10 and comprising the formula: 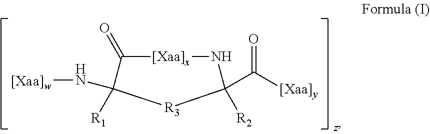
or a pharmaceutically acceptable salt thereof;
wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
wherein each R₃ is independently alkane alkylene, alkenylene, or alkynylene, any of which is substituted or unsubstituted;
wherein z is 1, 2, 3, 4, 5, 6, 7, 8, 9, or 10; and
wherein:
(i) each [Xaa]ₓ is KNITDK (SEQ ID NO: 55), or a variant thereof having one amino acid substitution;
(ii) each [Xaa]ₓ is NITDKI (SEQ ID NO: 58), or a variant thereof having one amino acid substitution;
(iii) each [Xaa]ₓ is ITDKID (SEQ ID NO: 61), or a variant thereof having one amino acid substitution;
(iv) each [Xaa]ₓ is TDKIDQ (SEQ ID NO: 64), or a variant thereof having one amino acid substitution;
(v) each [Xaa]ₓ is DKIDQI (SEQ ID NO: 67), or a variant thereof having one amino acid substitution;
(vi) each [Xaa]ₓ is KIDQII (SEQ ID NO: 70), or a variant thereof having one amino acid substitution;
(vii) each [Xaa]ₓ is IDQIIH (SEQ ID NO: 73), or a variant thereof having one amino acid substitution;
(viii) each [Xaa]ₓ is DQIIHD (SEQ ID NO: 76), or a variant thereof having one amino acid substitution; or
(ix) each [Xaa]ₓ is QIIHDF (SEQ ID NO: 79), or a variant thereof having one amino acid substitution;
wherein the conjugate binds to a 5 helix bundle or fusion bundle intermediate of EBOV GP2 and/or wherein the conjugate inhibits infection of a cell by EBOV in pseudovirus and/or live EBOV virus assay and/or prevents infection of a cell by EBOV in a pseudovirus and/or a live EBOV virus assay; and
optionally wherein the structurally-stabilized peptide is 28 to 40 amino acids in length, optionally 28 to 35 amino acids in length.

2. The conjugate of claim 1, comprising PEG and cholesterol.

3. The conjugate of claim 1, comprising PEG and thiocholesterol.

4. The conjugate of claim 2, wherein the conjugate comprises PEG(n)-cholesterol, wherein n is 1-36, optionally wherein n is 4, 5, 6, 7, or 8.

5. The conjugate of claim 3, wherein the conjugate comprises PEG(n)-thiocholesterol, wherein n is 2-36, optionally wherein n is 4, 5, 6, 7, or 8.

6. The conjugate of claim 3, wherein the PEG and thiocholesterol comprises the formula:

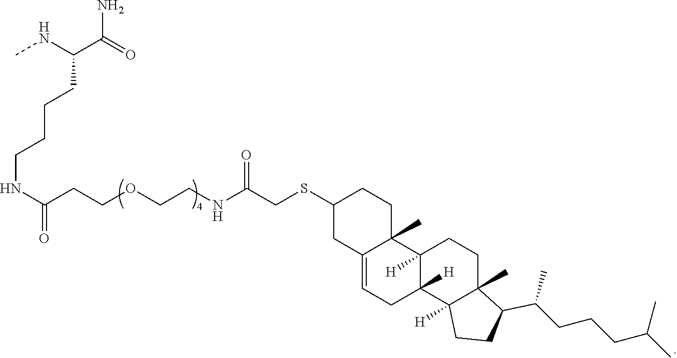

7. The conjugate of claim 2, wherein the PEG and cholesterol comprises the formula:

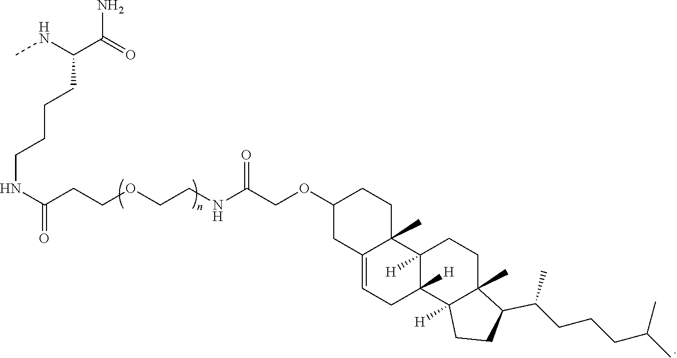

8. The conjugate of any one of claims 1 to 7, wherein the conjugate comprises or consists of the amino acid sequence set forth in any one of SEQ ID NOs:30-38.

9. The conjugate of any one of claims 1 to 7, wherein the conjugate comprises or consists of the amino acid sequence set forth in any one of SEQ ID NOs:39-47.

10. The conjugate of claim 1, wherein the structurally-stabilized peptide comprises or consists of the amino acid sequence set forth in any one of SEQ ID NOs:12-20.

11. The conjugate of claim 1, wherein the structurally-stabilized peptide comprises or consists of the amino acid sequence set forth in any one of SEQ ID NOs:21-29.

12. A structurally-stabilized peptide comprising an amino acid sequence comprising 28-32 contiguous amino acids of the sequence set forth in SEQ ID NO:10 with two to five amino acid substitutions relative to the sequence set forth in SEQ ID NO:10;
wherein two of the two to five amino acid substitutions are with α, α-disubstituted non-natural amino acids with olefinic side chains cross-linked to each other at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32,
wherein the structurally-stabilized peptide binds to a 5 helix bundle or fusion bundle intermediate of EBOV GP2 and/or wherein the structurally-stabilized peptide inhibits infection of a cell by EBOV in pseudovirus and/or live EBOV virus assay and/or prevents infection of a cell by EBOV in a pseudovirus and/or a live EBOV virus assay; and
optionally wherein the structurally-stabilized peptide is 28 to 40 amino acids in length, optionally 28 to 35 amino acids in length.

13. The structurally-stabilized peptide of claim 12, which comprises or consists of the amino acid sequence set forth in any one of SEQ ID NOs: 30-38.

14. The structurally-stabilized peptide of claim 12, which comprises or consists of the amino acid sequence set forth in any one of SEQ ID NOs: 39-47.

15. A structurally-stabilized peptide comprising an amino acid sequence comprising 28-32 contiguous amino acids of the sequence set forth in SEQ ID NO:10 with two to five amino acid substitutions relative to the sequence set forth in SEQ ID NO:10 and comprising the formula: 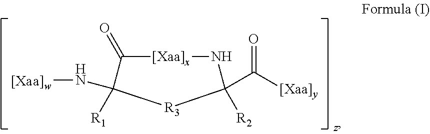
or a pharmaceutically acceptable salt thereof;
wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
wherein each R₃ is independently alkane alkylene, alkenylene, or alkynylene, any of which is substituted or unsubstituted;
wherein z is 1, 2, 3, 4, 5, 6, 7, 8, 9, or 10; and
wherein:
(i) each [Xaa]ₓ is KNITDK (SEQ ID NO: 55), or a variant thereof having one amino acid substitution;
(ii) each [Xaa]ₓ is NITDKI (SEQ ID NO: 58), or a variant thereof having one amino acid substitution;
(iii) each [Xaa]ₓ is ITDKID (SEQ ID NO: 61), or a variant thereof having one amino acid substitution;
(iv) each [Xaa]ₓ is TDKIDQ (SEQ ID NO: 64), or a variant thereof having one amino acid substitution;
(v) each [Xaa]ₓ is DKIDQI (SEQ ID NO: 67), or a variant thereof having one amino acid substitution;
(vi) each [Xaa]ₓ is KIDQII (SEQ ID NO: 70), or a variant thereof having one amino acid substitution;
(vii) each [Xaa]ₓ is IDQIIH (SEQ ID NO: 73), or a variant thereof having one amino acid substitution;
(viii) each [Xaa]ₓ is DQIIHD (SEQ ID NO: 76), or a variant thereof having one amino acid substitution; or
(ix) each [Xaa]ₓ is QIIHDF (SEQ ID NO: 79), or a variant thereof having one amino acid substitution;
wherein the structurally-stabilized peptide binds to a 5 helix bundle or fusion bundle intermediate of EBOV GP2 and/or wherein the structurally-stabilized peptide inhibits infection of a cell by EBOV in pseudovirus and/or live EBOV virus assay and/or prevents infection of a cell by EBOV in a pseudovirus and/or a live EBOV virus assay;
optionally wherein the structurally-stabilized peptide is 28 to 40 amino acids in length, optionally 28 to 35 amino acids in length.

16. The structurally-stabilized peptide of claim 15, wherein:
(i) each [Xaa]_{w} is TCHILGPDCAIEPHDW (SEQ ID NO:54), [Xaa]ₓ is KNITDK (SEQ ID NO: 55), and each [Xaa]_{y} is DQIIHDFV (SEQ ID NO:56);
(ii) each [Xaa]_{w} is TCHILGPDCAIEPHDWT (SEQ ID NO:57), [Xaa]ₓ is NITDKI (SEQ ID NO: 58), and each [Xaa]_{y} is QIIHDFV (SEQ ID NO:59);
(iii) each [Xaa]_{w} is TCHILGPDCAIEPHDWTK (SEQ ID NO:60), [Xaa]ₓ is ITDKID (SEQ ID NO: 61), and each [Xaa]_{y} is IIHDFV (SEQ ID NO:62);
(iv) each [Xaa]_{w} is TCHILGPDCAIEPHDWTKN (SEQ ID NO:63), [Xaa]ₓ is TDKIDQ (SEQ ID NO: 64), and each [Xaa]_{y} is IHDFV (SEQ ID NO:65);
(v) each [Xaa]_{w} is TCHILGPDCAIEPHDWTKNI (SEQ ID NO:66), [Xaa]ₓ is DKIDQI (SEQ ID NO: 67), and each [Xaa]_{y} is HDFV (SEQ ID NO:68);
(vi) each [Xaa]_{w} is TCHILGPDCAIEPHDWTKNIT (SEQ ID NO:69), [Xaa]ₓ is KIDQII (SEQ ID NO: 70), and each [Xaa]_{y} is DFV;
(vii) each [Xaa]_{w} is TCHILGPDCAIEPHDWTKNITD (SEQ ID NO:72), [Xaa]ₓ is IDQIIH (SEQ ID NO: 73), and each [Xaa]_{y} is FV;
(viii) each [Xaa]_{w} is TCHILGPDCAIEPHDWTKNITDK (SEQ ID NO:75), [Xaa]ₓ is DQITIHD (SEQ ID NO: 76), and each [Xaa]_{y} is V; or
(ix) each [Xaa]_{w} is TCHILGPDCAIEPHDWTKNITDKI (SEQ ID NO:77), [Xaa]ₓ is QIIHDF (SEQ ID NO: 78), and each [Xaa]_{y} is absent.

17. A pharmaceutical composition comprising the conjugate of any one of claims 1 to 11 or the structurally-stabilized peptide of any one of claims 12 to 16, and a pharmaceutically acceptable carrier.

18. A method of treating an ebolavirus infection in a subject in need thereof, the method comprising administering to the subject a therapeutically-effective amount of the conjugate of any one of claims 1 to 11 or of the structurally-stabilized peptide of any one of claims 12 to 16.

19. A method of preventing an ebolavirus infection in a subject in need thereof, the method comprising administering to the subject a therapeutically-effective amount of the conjugate of any one of claims 1 to 11 or of the structurally-stabilized peptide of any one of claims 12 to 16.

20. The method of claim 18 or 19, wherein the subject is a human.

21. A method of making a structurally-stabilized peptide, the method comprising:
(a) providing a peptide having an amino acid sequence comprising 28-32 contiguous amino acids of the sequence set forth in SEQ ID NO:10 with two to five amino acid substitutions relative to the sequence set forth in SEQ ID NO:10;
wherein two of the two to five amino acid substitutions are with α, α-disubstituted non-natural amino acids with olefinic side chains at positions of the sequence set forth in SEQ ID NO: 10 selected from (wherein position 1 is the N-terminal threonine and position 32 is the C-terminal valine of SEQ ID NO: 10):
(i) positions 17 and 24,
(ii) positions 18 and 25,
(iii) positions 19 and 26,
(iv) positions 20 and 27,
(v) positions 21 and 28,
(vi) positions 22 and 29,
(vii) positions 23 and 30,
(viii) positions 24 and 31, or
(ix) positions 25 and 32; and
(b) cross-linking the peptide, and optionally purifying the structurally-stabilized peptide.

22. The method of claim 21, wherein the cross-linking is by a ruthenium catalyzed metathesis reaction.

23. The method of claim 21 or 22, further comprising derivatizing a resin bound amine of the structurally-stabilized peptide with PEG and/or cholesterol containing a carboxylic acid on a resin.

24. The method of any one of claims 21 to 23, further comprising formulating the structurally-stabilized peptide as a sterile pharmaceutical composition.

25. The conjugate of any one of claims 1 to 7, wherein the conjugate comprises or consists of the amino acid sequence set forth in any one of SEQ ID NOs:80-92.

26. The conjugate of any one of claims 1 to 7, wherein the conjugate comprises or consists of the amino acid sequence set forth in any one of SEQ ID NOs:93-105.

27. The conjugate of claim 1, wherein the structurally-stabilized peptide comprises or consists of the amino acid sequence set forth in any one of SEQ ID NOs:106-118.

28. The conjugate of claim 1, wherein the structurally-stabilized peptide comprises or consists of the amino acid sequence set forth in any one of SEQ ID NOs:119-131.

29. The structurally-stabilized peptide of claim 12, which comprises or consists of the amino acid sequence set forth in any one of SEQ ID NOs: 80-92.

30. The structurally-stabilized peptide of claim 12, which comprises or consists of the amino acid sequence set forth in any one of SEQ ID NOs: 93-105.

31. A pharmaceutical composition comprising the conjugate of any one of claims 25 to 28 or the structurally-stabilized peptide of claim 29 or 30, and a pharmaceutically acceptable carrier.

32. A method of treating an ebolavirus infection in a subject in need thereof, the method comprising administering to the subject a therapeutically-effective amount of the conjugate of any one of claims 25 to 28 or the structurally-stabilized peptide of claim 29 or 30.

33. A method of preventing an ebolavirus infection in a subject in need thereof, the method comprising administering to the subject a therapeutically-effective amount of the conjugate of any one of claims 25 to 28 or the structurally-stabilized peptide of claim 29 or 30.

34. A method of treating a Marburg virus infection, a Bombali ebolavirus infection, or a Mengla dianlovirus infection in a subject in need thereof, the method comprising administering to the subject a therapeutically-effective amount of the conjugate of any one of claims 1 to 11 and 25 to 28 or of the structurally-stabilized peptide of any one of claims 12 to 16, 29, and 30.

35. A method of preventing a Marburg virus infection, a Bombali ebolavirus infection, or a Mengla dianlovirus infection in a subject in need thereof, the method comprising administering to the subject a therapeutically-effective amount of the conjugate of any one of claims 1 to 11 or of the structurally-stabilized peptide of any one of claims 12 to 16, 29, and 30.

36. The method of claim 34 or 35, wherein the subject is a human.
